(19) United States
(12) Patent Application Publication
(10) Pub. No.: US 2008/0131400 A1
(43) Pub. Date: Jun. 5, 2008

(54) Vector system

(76) Inventors: Nicholas Mazarakis, Oxford (GB); Mimoun Azzouz, Oxford (GB); Susan Mary Kingsman, Oxford (GB)

Correspondence Address: FROMMER LAWRENCE & HAUG, 745 FIFTH AVENUE- 10TH FL., NEW YORK, NY, 10151, US

(21) Appl. No.: 11/810,007

(22) Filed: Jun. 4, 2007

Related U.S. Application Data
(63) Continuation of application No. 10/716,725, filed on Nov. 19, 2003, now abandoned.
Application No. 10/716,725 is a continuation-in-part of application No. 10/429,608, filed on May 5, 2003, now abandoned.
Application No. 10/429,608 is a continuation-in-part of application No. PCT/GB01/04866, filed on Nov. 2, 2001.
Application No. PCT/GB01/04866 is a continuation-in-part of application No. PCT/GB03/00426, filed on Oct. 3, 2003.

(30) Foreign Application Priority Data
Nov. 3, 2000 (GB) 0026943.1
Jan. 30, 2001 (GB) 0102339.9
Sep. 14, 2001 (GB) 0122238.9
Oct. 4, 2002 (GB) 0223076.1
Dec. 4, 2002 (GB) 0228314.1
Aug. 4, 2003 (GB) 0318213.6

(51) Int. Cl. A61K 35/76; A61P 25/00

(52) U.S. Cl. 424/93.2

(57) ABSTRACT

Provided is a method of treating motor neuron disease using a lentiviral vector system to transduce a target site, wherein the vector system is or comprises at least part of a rabies G envelope protein or a mutant, variant, homologue or fragment thereof, and a nucleotide of interest (NOI), and wherein the target site is at least part of the central nervous system.

### CROSS REFERENCE TO RELATED APPLICATIONS

This application is a continuation-in-part of U.S. application Ser. No. 10/429,608, filed on May 5, 2003, which is a continuation-in-part of International application no. PCT/GB01/04866, filed on Nov. 2, 2001, designating the U.S., published on May 10, 2002 as WO 02/36170, and claiming priority from GB application nos. 0026943.1, filed on Nov. 3, 2000, 0102339.9, filed on Jan. 30, 2001 and 0122238.9 filed on Sep. 14, 2001. This application is also a continuation-in-part of International application no. PCT/GB03/00426, filed on Oct. 3, 2003, and claiming priority from GB application nos. 0223076.1, filed on Oct. 4, 2002, 0228314.1, filed on Dec. 4, 2002 and 0318213.6, filed on Aug. 4, 2003. This application makes reference to U.S. application Ser. No. 09/701,014, filed on Nov. 22, 2000, which is an application under 35 U.S.C. §371 from International application no. PCT/GB99/01607, filed on May 21, 1999, claiming priority to U.S. application Ser. No. 60/093,149, filed on Jul. 17, 1998 and UK application no. 9811153.7, filed on May 22, 1998. This application also makes reference to U.S. application Ser. No. 10/408,456, filed on Apr. 7, 2003, which is a CIP of International application no. PCT/GB01/04433, filed on Oct. 5, 2001, designating the U.S., published on Apr. 11, 2002 as WO 02/29065, and claiming priority from GB 0024550.6, filed on Oct. 6, 2000. This application also makes reference to U.S. application Ser. No. 10/239,804, filed on Sep. 23, 2002, which is an application under 35 U.S.C. §371 from International application no. PCT/GB01/01478, filed on Mar. 30, 2001, claiming priority to UK application no. 0024300.6, filed on Oct. 4, 2000, and to International application no. PCT/GB00/01211, filed on Mar. 30, 2000, which claims priority to UK application no. 9907461.9, filed on Mar. 31, 1999. This application also makes reference to U.S. application Ser. No. 09/937,716, filed on Jul. 1, 2002, which is an application under 35 U.S.C. §371 from International application no. PCT/GB00/01211, filed on Mar. 30, 2000, which claims priority to UK application no. 9907461.9, filed on Mar. 31, 1999.

All of the foregoing applications, as well as all documents cited in the foregoing applications (“application documents”) and all documents cited or referenced in the application documents are incorporated herein by reference. Also, all documents cited in this application (“herein-cited documents”) and all documents cited or referenced in herein-cited documents are incorporated herein by reference. In addition, any manufacturer's instructions or catalogues for any products cited or mentioned in each of the application documents or herein-cited documents are incorporated by reference. Documents incorporated by reference into this text or any teachings therein can be used in the practice of this invention. Documents incorporated by reference into this text are not admitted to be prior art.

### FIELD OF THE INVENTION

The present invention relates to a vector system. In particular, the present invention relates to a vector system capable of delivering an entity of interest (“EOI”)—such as a nucleotide sequence of interest (“NOI”)—to a target site, such as for the treatment of diseases affecting the central nervous system (CNS).

In one preferred aspect, the present invention relates to a viral vector system capable of delivering a nucleotide sequence of interest (“NOI”) to a target site. The target site can be a neuron, for example. In an especially preferred aspect, the viral vector system is a lentiviral vector system.

In another preferred aspect, the present invention relates to a vector system capable of travelling to a target site by retrograde transport. In particular, the present invention relates to the use of such a vector system to transduce distal, connected sites within the nervous system.

More in particular, the present invention relates to a retroviral vector useful in gene therapy.

### BACKGROUND OF THE INVENTION

Gene therapy includes any one or more of: the addition, the replacement, the deletion, the supplementation, the manipulation etc. of one or more nucleotide sequences in, for example, one or more targeted sites—such as targeted cells. If the targeted sites are targeted cells, then the cells may be part of a tissue or an organ. General teachings on gene therapy may be found in Molecular Biology (Ed Robert Meyers, Pub VCH, such as pages 556-558).

By way of further example, gene therapy also provides a means by which any one or more of: a nucleotide sequence, such as a gene, can be applied to replace or supplement a defective gene; a pathogenic gene or gene product can be eliminated; a new gene can be added in order, for example, to create a more favourable phenotype; cells can be manipulated at the molecular level to treat cancer (Schmidt-Wolf and Schmidt-Wolf, 1994, Annals of Hematology 69; 273-279) or other conditions—such as immune, cardiovascular, neurological, inflammatory or infectious disorders; antigens can be manipulated and/or introduced to elicit an immune response—such as genetic vaccination.

In recent years, retroviruses have been proposed for use in gene therapy. Essentially, retroviruses are RNA viruses with a life cycle different to that of lytic viruses. In this regard, when a retrovirus infects a cell, its genome is converted to a DNA form. In other words, a retrovirus is an infectious entity that replicates through a DNA intermediate. More details on retroviral infection etc. are presented later on.

With regard to the genetic structure of a viral vector, the gene env encodes the surface (SU) glycoprotein and the transmembrane (TM) protein of the virion, which form a complex that interacts specifically with cellular receptor proteins. This interaction leads ultimately to fusion of the viral membrane with the cell membrane.

Although uncleaved Env proteins are able to bind to the receptor, the cleavage event itself is necessary to activate the fusion potential of the protein, which is necessary for entry of the virus into the host cell. Typically, both SU and TM proteins are glycosylated at multiple sites. However, in some viruses, exemplified by MLV, TM is not glycosylated.

Although the SU and TM proteins are not always required for the assembly of enveloped virion particles as such, they do play an essential role in the entry process. In this regard, the SU domain binds to a receptor molecule—often a specific receptor molecule—on the target cell. It is believed that this binding event activates the membrane fusion-inducing potential of the TM protein after which the viral and cell membranes fuse. In some viruses, notably MLV, a cleavage event—resulting in the removal of a short portion of the cytoplasmic tail of TM—is thought to play a key role in uncovering the full fusion activity of the protein (Brody et al. 1994 J. Virol. 68: 4620-4627, Rein et al. 1994 J. Virol. 68: 1773-1781). This cytoplasmic “tail”, distal to the membrane-spanning segment of TM remains on the internal side of the viral membrane and it varies considerably in length in different retroviruses.

Thus, the specificity of the SU/receptor interaction can define the host range and tissue tropism of a retrovirus. In some cases, this specificity may restrict the transduction potential of a recombinant retroviral vector. For this reason, many gene therapy experiments have used MLV. A particular MLV that has an envelope protein called 4070A is known as an amphotropic virus, and this can also infect human cells because its envelope protein “docks” with a phosphate transport protein that is conserved between man and mouse. This transporter is ubiquitous and so these viruses are capable of infecting many cell types. In some cases however, it may be beneficial, especially from a safety point of view, to specifically target restricted cells. To this end, several groups have engineered a mouse ecotropic retrovirus, which unlike its amphotropic relative normally only infects mouse cells, to specifically infect particular human cells. Replacement of a fragment of an envelope protein with an erythropoietin segment produced a recombinant retrovirus which then bound specifically to human cells that expressed the erythropoietin receptor on their surface, such as red blood cell precursors (Maulik and Patel 1997 “Molecular Biotechnology: Therapeutic Applications and Strategies” 1997. Wiley-Liss Inc. pp 45.).

Replacement of the env gene with a heterologous env gene is an example of a technique or strategy called pseudotyping. Pseudotyping can confer one or more advantages. For example, with the lentiviral vectors, the env gene product of the HIV based vectors would restrict these vectors to infecting only cells that express a protein called CD4. But if the env gene in these vectors has been substituted with env sequences from other RNA viruses, then they may have a broader infectious spectrum (Verma and Somia 1997 Nature 389:239-242).

More generally, delivery of therapeutic molecules to the CNS represents an important challenge for the treatment of neurodegenerative diseases. Limitations to overcome include (i) the presence of the blood-brain barrier, (ii) side effects associated with systemic administration, and (iii) instability of the molecules.

One problem with gene therapy approaches in the treatment of, for example, Parkinson's disease, is that brain is a difficult and complex organ to target (Raymon H. K. et al. (1997) Exp. Neur. 144: 82-91). The usual route is by injection of vectors to the striatum (Bilang-Bleuel et al. (1997) Proc. Acad. Natl. Sci. USA 94:8818-8823; Choi-Lundberg et al. (1998) Exp. Neurol. 154:261-275) or to near the substantia nigra (Choi-Lundberg et al. (1997) Science 275:838-841; Mandel et al. (1997) Proc. Acad. Natl. Sci. USA 94:14083-14088). It is technically difficult to inject directly into the some parts of the brain, for example because of their location and/or size. The substantia nigra lies deep in the brain and direct injection to this area can cause lesion of axons, resulting in damage. The striatum, in particular the caudate putamen, is a relatively easy target because it is larger and more dorsal than the substantia nigra. It has been used extensively for transplantation in Parkinson's disease, and there is currently thought to be less than 1% risk involved in the operation. Similar problems exist in relation to other parts of the CNS.

Hence, it is desirable to find a mechanism for transducing parts of the brain and other parts of the CNS which are difficult to reach by direct injection. It is also desirable to find an administration strategy for cranial gene therapy which minimises the number and complexity of brain injections. It is also desirable to achieve good penetration and distribution throughout the nervous system following administration.

An optimal method of transducing cells within the CNS will obviate the need to cross the blood-brain barrier, target the required group of cells, and avoid damaging CNS tissue during administration.

It has been thought that pseudotyping might alleviate some of the above-mentioned problems. However, the transduction and expression characteristics of pseudotyped vectors have not yet been fully determined and there remains the need to provide further and improved vectors.

By way of example, Mazarakis et al. (2001) Human Molecular Genetics 10(19):2109-2121 teaches that a lentiviral vector pseudotyped with VSV G transduced muscle cells surrounding an injection site, but did not result in expression in any cells in the spinal cord.

WO02/36170 teaches the use of a wild-type rabies G protein to achieve retrograde transport, and particularly transduction of a TH positive neuron. We have found that it is possible to achieve good biodistribution of an entity of interest (EOI) through a mechanism other than retrograde transport using rabies G proteins. Thus, it will be appreciated that this enables sites to be targeted through administration sites other than those which would be available using the retrograde transport mechanism. Whilst not wishing to be bound by any theory we believe that this high level of distribution may be achieved through a diffusion mechanism. In contrast, we have found that VSV G pseudotyping does not give rise to such biodistribution confirming the surprising result demonstrated herein. It will be appreciated that good biodistribution is important so that different parts of the central nervous system can be accessed through a localised site of administration. This particularly helps where penetration by an EOI to sites which are not readily accessible is required. We have also found that pseudotyped EIAV vectors give a particularly good effect.

We have also found that retrograde transport and transduction of cells of the CNS can be achieved using the rabies G protein from Challenge Virus Standard (CVS). We believe that we are the first to demonstrate the advantages of lentiviral pseudotyping with a CVS protein.

In addition, we have found that pseudotyping with rabies G proteins such as CVS envelope proteins give particular advantages when administered in utero or to a neonate. In these circumstances we have found that one can achieve good transduction in muscle cells, which is surprising given that transduction is poor in adult cells. We have also found that transport, e.g. by retrograde transport, to motor and sensory neurons is enhanced. These results are particularly advantageous where therapy needs to be administered in the early stages of life, e.g. in the case of spinal muscular atropy.

### SUMMARY OF THE INVENTION

In a broad aspect, the present invention relates to a vector system that is capable of causing retrograde transport of an entity of interest (“EOI”).

As used herein the term “vector system” includes any vector that is capable of infecting or transducing or transforming or modifying a recipient cell with an EOI.

The EOI may be a chemical compound, a biological compound or combinations thereof. By way of example, the EOI may be a protein (such as a growth factor), a nucleotide sequence, an organic and/or an inorganic pharmaceutical (such as an analgesic, an anti-inflammatory, a hormone, a lipid), or combinations thereof.

The vector system of the present invention is capable of delivering the EOI to a site, wherein at that site the EOI may then be distributed and/or penetrate distant sites, e.g. through diffusion or retrograde transport.

Typically the vector system will also comprise an EOI, preferable an NOI. The NOI preferably encodes a neurotrophic or anti-apoptotic gene. In a further preferred embodiment, the NOI encodes SMN-1, GDNF, IGF-1, VEGF, XIAP, NLAP, bcl-2, or RARβ2.

According to one aspect of the present invention there is provided a method of treating motor neuron disease in a patient in need thereof, the method comprising delivering to a target site, a lentiviral vector pseudotyped with a rabies G envelope protein or a mutant, variant, homologue or fragment thereof, the lentiviral vector comprising an NOI, wherein the target site is at least part of the central nervous system, and wherein the NOI encodes a gene product that is expressed in the target site, thereby treating motor neuron disease in the patient.

In one embodiment, treatment of the motor neuron disease comprises halting or delaying the degeneration of motor neurons in the patient. Preferably, the motor neuron disease is ALS (Amyotrophic Lateral Sclerosis) or SMA (Spinal Muscular Atrophy).

According to another aspect of the present invention there is provided a method of delivering an NOI to a target site, comprising introducing a lentiviral vector comprising an NOI and pseudotyped with a rabies G envelope protein or a mutant, variant, homologue or fragment thereof, to the target site, wherein the target site is at least part of the central nervous system.

According to yet another aspect of the present invention there is provided a method of expressing an NOI in a target site, comprising introducing a lentiviral vector comprising an NOI and pseudotyped with a rabies G envelope protein or a mutant, variant, homologue or fragment thereof, to the target site, wherein the target site is at least part of the central nervous system, and wherein the NOI encodes a gene product that is expressed in the target site.

According to a further aspect of the present invention there is provided use of a vector system to transduce an in utero target site or a target site in a neonate, wherein the vector system is or comprises at least part of a rabies G envelope protein or a mutant, variant, homologue or fragment thereof.

The target site is preferably a target cell selected from the group consisting of a sensory neuron, a motor neuron, an astrocyte, an oligodendrocyte, a microglial cell, and an ependymal cell.

There are a variety of methods for introducing the lentiviral vector comprising the NOI to the target site, for example, by diffusion or retrograde transport. The lentiviral vector comprising the NOI can be delivered via intramuscular or intraparenchymal administration.

The vector system can be a non-viral system or a viral system, or combinations thereof. In addition, the vector system itself can be delivered by viral or non-viral techniques.

Viral vector or viral delivery systems include but are not limited to adenoviral vectors, adeno-associated viral (AAV) vectors, herpes viral vectors, retroviral vectors, lentiviral vectors, and baculoviral vectors. Non-viral delivery or non-viral vector systems include lipid mediated transfection, liposomes, immunoliposomes, lipofectin, cationic facial amphiphiles (CFAs) and combinations thereof.

In non-viral vector systems of the present invention, the at least part of the rabies G protein (or a mutant, variant, homologue or fragment thereof) may be used to encapsulate or enshroud an EOI. Thus, for some embodiments, the at least part of the rabies G protein (or a mutant, variant, homologue or fragment thereof) may form a matrix around the EOI. Here, the matrix may contain other components—such as a liposome type entity.

In some preferred aspects, the vector system is a viral vector system.

In some further preferred aspects, the vector system is a retroviral vector system and, preferably, a lentiviral vector system.

It has also been found that a particular type of vector system—such as a viral vector system, preferably a retroviral vector system, more preferably a lentiviral vector system—according to the present invention is capable of transducing one or more sites which are distant from the site of administration due to retrograde transport of the vector system.

Administration to a single target site may cause transduction of a plurality of target sites. The vector system may travel to the or each target site by retrograde transport, diffusion or biodistribution, optionally in combination with anterograde transport.

In further broad aspects, the present invention relates to:

(i) a method of treating and/or preventing a diseases using such a vector system;

(ii) the use of such a vector system in the manufacture of a pharmaceutical composition to treat and/or prevent a disease;

(iii) a method for analysing the effect of a protein of interest in a cell using such a vector system;

(iv) a method for analysing the function of a gene or protein using such a vector system;

(v) a cell transduced with such a vector system;

(vi) an immortalised cell made by transduction with such a vector system;

(vii) the use of such an immortalised cell in the manufacture of a medicament; and

(viii) a transplantation method using such an immortalised cell.

In further preferred embodiments, the present invention relates to:

(i) The use of a lentiviral vector comprising a nucleotide of interest (NOI) in the manufacture of a medicament to deliver an NOI to a target site, wherein the lentiviral vector is pseudotyped with a rabies G envelope protein; and the target site is at least part of the central nervous system; and

(ii) The use of a lentiviral vector comprising a nucleotide of interest (NOI) in the manufacture of a medicament to express an NOI in a target site, wherein the lentiviral vector is pseudotyped with a rabies G envelope protein; the target site is at least part of the central nervous system; and the NOI encodes a gene product that is expressed in the target site.

### BRIEF DESCRIPTION OF THE DRAWINGS

The following Detailed Description, given by way of example, but not intended to limit the invention to specific embodiments described, may be understood in conjunction with the accompanying drawings, incorporated herein by reference. Various preferred features and embodiments of the present invention will now be described by way of non-limiting example and with reference to the accompanying drawings in which:

FIGS. 1A-1D show the expression of EIAV (pONY8 GFP) Rabies-G viral vector in TH+ neurons of mouse E14 mesencephalic cultures. FIG. 1A shows an image of GFP+ neuron on top of a layer of transduced astrocytes (flat cells slightly out of focus). FIG. 1B shows an image of the same neuron also staining for TH. Transduction for 1A and 1B is at an MOI of 1. FIG. 1C shows an image of GFP+ neurons on top of astrocytes. FIG. 1D shows that two of these GFP neurons also stain for TH although others are clearly negative. None of the glia stain with TH. Transduction for 1C and 1D is at an MOI of 10.

FIGS. 2A-2F show the expression of EIAV (pONY8 GFP) Rabies-G viral vector in glia and TH-neurons in mouse E14 mesencephalic cultures. FIGS. 2A and 2B show the same field in which several GFP+ neurons (2A) could be found that are TH− (2B). FIGS. 2C and 2D show the same field of control cells treated only with polybrene and no virus expressing TH (2D) but not GFP. FIGS. 2E and 2F show the same field of cells, wherein a clump of GFP+ astrocytes (2E) express no TH (2F). MOI for these transductions is 1.

FIGS. 3A-3H show the effect of transduction of the adult rat striatum with EIAV pONY8Z VSVG viral vector (1 week post-injection). FIGS. 3A-3C correspond to 3 independent 50 μm coronal sections stained with X-gal. An average of fifty of such sections are stained per animal, indicating that the transduction spans the rat striatum. FIGS. 3D-3H represent higher magnification of the section in FIG. 3C, showing that many of the cells transduced have neuronal morphology both within caudate putamen (3D-3F) and in nucleus accumbens (3G-3H).

FIGS. 4A-4F show cell types transduced in the adult rat striatum with EIAV pONY8Z VSVG viral vector. FIGS. 4A-4C show high magnification images of striatal neurons; larger aspiny interneurons (4A, 4B) and medium-sized spiny neurons (4C) are stained. LacZ expressing cells, shown in FIG. 4D, colocalised with the neuronal postmitotic marker NeuN, shown in FIG. 4E, giving bright nuclear staining, shown in FIG. 4F.

FIGS. 5A-5C show the transduction of globus pallidus and reticular thalamic nucleus. FIG. 5A shows that, in rats where transduction with EIAV pONY8Z VSVG spread to lateral globus pallidus (LGP), LacZ staining is also observed in thalamic reticular nucleus (RTN). Higher magnification views, shown in FIGS. 5B and 5C, indicate the presence of efferent connections from GP passing along the zona incerta to RTN and thalamus. This anterograde transport is reported in other studies using specific anterograde tracers (Shammah-Lagnado et al J Comp Neurol 1996 376: 489-507).

FIGS. 6A-6D show the transduction of the adult rat striatum with EIAV pONY8Z RabiesG viral vector. FIG. 6A shows a low magnification view of brain section showing transduction in caudate adjacent to lateral ventricle. Higher magnifications of the same section, shown in FIGS. 6B-6D, demonstrate the punctate nature of expression (6B) and transduction of cells with astroglial morphology (6C arrows) as well as neuronal morphology (6D arrow).

FIGS. 7A-7H show the transduction of neuronal nuclei distant to the area of injection after delivery of EIAV pONY8Z RabiesG viral vector in adult rat striatum (8 days post-injection). FIG. 7A is a low magnification image of brain section showing transduction in globus pallidus (LGP) and paraventricular nuclei of thalamus (PVT). FIG. 7B is a higher magnification image of transduced pallidal neurons. FIG. 7C is a low magnification image of brain section showing staining in paraventricular paracentral nucleus of stria terminalis and also staining in amygdala (ventral). FIG. 7D is a higher magnification image of FIG. 7A, with punctate staining of paraventricular nucleus of thalamus. FIG. 7E is a higher magnification of FIG. 7C, showing staining of neurons in the amygdala. FIG. 7F shows stria terminalis staining in paraventricular nucleus thalamus. FIG. 7G shows hypothalamic neurons of the paraventricular nucleus staining adjacent to the third ventricle. FIG. 7H shows neuronal staining in SN reticulata. Thalamic staining implies retrograde transport of viral particles from neuronal terminals to neuronal cell bodies.

FIGS. 8A-8F show long-term expression of LacZ after transduction of the adult rat striatum with EIAV pONY8Z RabiesG viral vector. FIGS. 8A and 8D show striatal staining. FIG. 8B shows staining in parafascicular nucleus of thalamus (PFN) and weaker staining in subthalamic nucleus. FIG. 8C shows staining in SN compacta and reticulata; FIG. 8E shows neuronal staining in globus pallidus; and FIG. 8F shows punctate staining of medial thalamic nuclei. FIGS. 8A-8C show expression after 3 months, while FIGS. 8D-8F show expression 6 months postinjection. Thalamic and SNc staining implies retrograde transport of viral particles from neuronal terminals to neuronal cell bodies.

FIGS. 9A-9D show the transduction of the adult rat substantia nigra with EIAV pONY8Z VSVG viral vector. FIG. 9A is a low magnification image showing spread of transduction after perinigral injection both in SNc, medial thalamus and hypothalamus. FIG. 9B is a higher magnification image showing neuronal transduction of thalamus with commissural neurons (CN) whose labelled axons cross dorsal to the third ventricle (3V) and terminate in contralateral thalamus. LacZ is transported in an anterograde manner in this case. FIGS. 9C and 9D are higher magnification images of transduction of SNc showing stained neural projections from SNc to SNr. Transduction was 4 weeks postinjection.

FIGS. 10A and 10B show anterograde staining of nigrostriatal terminals after perinigral injection of EIAV pONY8Z VSVG. FIG. 10A is a low magnification image of brain striatal section from brain depicted in FIG. 9, showing LacZ staining of nigrostriatal terminals at the ipsilateral side of transduction. FIG. 10B is a higher magnification image of anterograde transport of LacZ, resulting in pale staining of neuronal terminals in striatum.

FIGS. 11A-11D show transduction of the adult rat substantia nigra with EIAV pONY8Z Rabies G viral vector. FIG. 11A shows strong staining of neurons within SNc and SNr. Also, extensive spreading is observed in thalamus dorsal to SN. FIG. 11B shows that transduction of ventral posterolateral (VPL) and ventral posteromedial thalamic nuclei (VPM), which receive input from medial lemniscus; centromedian nucleus (CM) and its thalamostriate fibers, which project to putamen; and STN, which projects to medial GP and receives input from LGP; was observed on the ipsilateral side injection. FIG. 11C shows punctate staining of putamen and cortex. Pale staining is indicative of neuronal terminals staining with LacZ transported anterogradely. FIG. 11D shows extensive transduction of neurons of globus pallidus (anterograde and retrograde transport). Transduction was 4 weeks postinjection.

FIGS. 12A-12B show staining after perinigral injection of EIAV pONY8Z Rabies G viral vector. FIG. 12A shows staining of cell bodies of central lateral (CLT) and parafascicular (PTN) thalamic nuclei, as well of the dorsal supraoptic decussation of the commissure of Maynert (DSC), with staining at the contalateral side from the injection. The commissure of Maynert projects from STN contalateral to the side of injection to globus pallidus on the ipsilateral side. Since GP is transduced, this staining implies retrograde transport of the vector to the neuronal bodies of the contalateral side. FIG. 12B shows staining of paraventricular nucleus of hypothalamus (PVH), as is also observed with VSVG pseudotyped vector (FIG. 7).

FIG. 13 shows a plasmid map of pONY8Z.

FIG. 14 shows a plasmid map of pONY8.0G.

FIGS. 15A-15M show gene transfer in primary neuronal cultures using EIAV lentiviral vectors. FIGS. 15A-15C show mouse E14 mesencephalic neurons infected with rabies-G pseudotyped pONY8.0G at an MOI of 10. A GFP expressing neuron from these cultures is shown in FIG. 15A labelled with an anti-GFP antibody, and in FIG. 15B with an anti-tyrosine hydroxylase (TH) antibody. FIG. 15C shows GFP and TH colocalisation in the merged confocal image. FIG. 15D shows that increasing the MOI leads to an increase in the number of neurons transduced, but no significant differences between the two pseudotypes are observed. FIG. 15E shows that there is no effect of transduction on ³H-DA release by mesencephalic neurons after lentiviral gene transfer is observed compared to control neurons. In FIGS. 15D, 15E, 15L and 15M, clear bars indicate cells infected with VSV-G pseudotyped vector; black bars indicate cells infected with rabies-G pseudotyped vector. FIGS. 15F-15H show rat E17 hippocampal neurons and FIGS. 15I-15K show striatal neurons infected with rabies pseudotyped EIAV vectors expressing β-gal at an MOI of 10. Cells are labelled with anti-β-gal (15F and 15I) and anti-Neuronal Nuclei (NeuN) antibodies (15G and 15J). FIGS. 15H and 15K are merged confocal images showing colocalization of the two antigens. As with the mesencephalic cultures, increasing MOI leads to an increase in the number of hippocampal (15L) and striatal (15M) neurons transduced. The “*” in FIGS. 15L and 15M indicates a significant increase in transduction efficiency with the rabies-G pseudotyped vector compared to the VSV-G pseudotype. Images 15A-15C and 15F-15K are at 60× magnification.

FIGS. 16A-16L show in vivo transduction of LacZ in the rat striatum with VSV-G (16A-16F) and rabies-G (16G-16L) pseudotyped pONY8Z vectors at one month post-injection. In FIG. 16A, extensive gene transfer at the site of injection in the caudate putamen is observed after VSV-G pseudotyped vector delivery, which is specific to the striatum and not to the fiber tracts transversing it. FIG. 16B is a higher magnification image of 16A, revealing cells with neuronal morphology close to the injection site (arrow). Anterograde transport of β-gal is observed in neuronal axons projecting from the injected striatum to anatomically linked projection sites, such as the lateral and medial globus pallidus, (16C and 16D), the cerebral penduncle adjacent to the subthalamic nucleus (FIG. 16E), and the substantia nigra pars reticulata (16F). The striatal projections to these sites are reviewed in (Parent et al. (2000) Trends Neurosci 23 S20-7). Some β-gal expressing cell bodies are observed only in the lateral globus pallidus, which implies that direct gene transfer has also occurred due to the proximity of this nucleus to the injection site. Gene transfer with rabies-G pseudotyped vectors in striatum leads to extensive β-gal staining in caudate putamen (16G and 16H) and also of the nearby globus pallidus (16I). Pallidal transduction leads to anterograde labelling of projections to thalamic reticular nucleus (16I). Labelling of these afferents was observed when anterograde tracers were placed in the globus pallidus. Retrograde transport of rabies-G pseudotyped viral vectors results in transduction of cell bodies in distal neuronal nuclei at anatomically connected sites including the amygdala (16I), several thalamic nuclei (16J and 16K), the subthalamic nucleus (16K) and the substantia nigra (16L). This phenomenon was not observed after similar delivery of VSV-G pseudotyped vectors.

FIGS. 16M-16U show confocal analysis of transduced cell-types in the rat striatum following injection of VSV-G (16M-16O) and rabies-G (16P-16U) pseudotyped EIAV viral vectors. Transduction is mainly neuronal in both cases, as demonstrated with β-gal (16M and 16P) and NeuN antibody staining (16N and 16Q) in the same sections. Colocalization of B-gal and NeuN expression can be seen in the merged images (16O and 16R). Note transduced striatal projection neuron is present in the case of VSV-G (arrow), but is absent in the striatum transduced with the rabies-G pseudotyped vector. In addition to neurons (arrow), rabies-G pseudotyped vector transduces astrocytes (16S-16U arrow), as demonstrated by anti-β-gal (16S) and anti-GFAP (16T) colocalisation (16U). Abbreviations: A: amygdala, CP: caudate putamen, cp: cerebral penduncle, CM: centromedial thalamic nucleus, ic: internal capsule, LGP: lateral globus pallidus, MGP: medial globus pallidus, PCN: pericentral thalamic nucleus, PF: perifasicular thalamic nucleus, SNc: substantia nigra pars compacta, SNr: substantia nigra pars reticulata, SMT: submedial thalamic nucleus, STh: subthalamic nucleus, TRN: thalamic reticular nucleus. FIGS. 16A, 16C-16G and 16I-16K are at 10× magnification; FIG. 16H is at 25× magnification; FIG. 16B is at 40× magnification; FIGS. 16M-16O are at 90× magnification; FIGS. 16P-16R are at 120× magnification; FIGS. 16S-16U are at 160× magnification.

FIGS. 17A-17C show reporter gene expression at eight months post-injection in the striatum and retrogradely transduced distal sites after striatal delivery of rabies-G pseudotyped pONY8Z vector. FIG. 17A shows strong expression at the site of delivery in the caudate putamen. Expression also remains strong at distal sites projecting to caudate putamen, such as the medial thalalamic nuclei (17B) and the substantia nigra (17C), which are transduced by retrograde transport of the rabies-G pseudotyped pONY8Z vector. Pale staining is observed in cerebral penduncle and substantia nigra pars reticulata from β-gal transported in axons of transduced striatal efferents. Abbreviations: CM: centromedial thalamic nucleus, CP: caudate putamen, cp: cerebral penduncle, PCN: pericentral thalamic nucleus, SMT: submedial thalamic nucleus, SNc: substantia nigra pars compacta, SNr: substantia nigra pars reticulata. FIGS. 17A and 17B are at 10× magnification; FIG. 17C is at 15× magnification.

FIGS. 17D-17I show confocal analysis showing retrogradely transduced neurons in globus pallidus (17D-17F) and substantia nigra pars compacta (17G-17I), after injection of rabies-G pseudotyped vector into the striatum. Micrographs demonstrate immunofluorescent labelling of neurons with anti-β-gal (17D and 17G), anti-NeuN (17E) and anti-tyrosine hydroxylase (17H) antibodies. Expression of β-gal colocalizes with the immunofluorescence of NeuN in pallidal neurons (17F) and tyrosine hydroxylase in nigral dopaminergic neurons (17I), producing bright staining. FIGS. 17D-17I are shown at 50× magnification.

FIG. 17J shows PCR analysis showing detection of EIAV vector DNA in thalamus and substantia nigra ipsilateral to the site of injection of the rabies-G pseudotyped vector in the rat striatum. Lane 1: 100 bp ladder; Lanes 2, 3, 4: Rat 1 (rabies-G pseudotyped vector) striatum, thalamus, substantia nigra; Lanes 5, 6, 7: Rat 2 (VSV-G pseudotyped vector) striatum, thalamus, substantia nigra; Lane 8: Rat 5 uninjected; Lane 9: water.

FIGS. 18A-18I show in vivo transduction of LacZ in the rat substantia nigra with VSV-G (18A-18C) and rabies-G (18D-18I) pseudotyped pONY8Z vectors at one month post-injection. In FIG. 18A, extensive gene transfer is observed with the VSV-G pseudotyped vector in the substantia nigra pars compacta and thalamus. FIG. 18B is a higher magnification of the substantia nigra showing extensive transduction of pars compacta neurons and their axons projecting to substantia nigra pars reticulata. FIG. 18C shows that β-gal protein is anterogradely transported to axon terminals of nigrostriatal neurons producing pale staining of ipsilateral striatum (encircled). Arrow in FIG. 18A indicates anterograde transport of β-gal and staining of commisural axons projecting to contralateral side, though no transduction of neuronal cell bodies was observed contralaterally. In FIG. 18D, extensive transduction of both substantia nigra and different thalamic nuclei is observed after delivery of rabies-G pseudotyped EIAV vectors. In this case, both substantia nigra pars compacta and substantia nigra pars reticulata are transduced (18E and 18F). Labelling of neurons in distal sites due to retrograde transport of this vector can be observed in lateral globus pallidus (18G and 18H), amygdala (18G) and commissural neurons projecting from contralateral thalamus (arrows, 18I). Anterograde transport of β-gal along axons is widespread, leading to staining of structures such as the thalamic reticular nucleus (18G), from lateral globus pallidus, and caudate putamen (18G and 18H), from substantia nigra pars compacta and lateral globus pallidus. Abbreviations: A: amygdala, APTD: anterior pretectal thalamic nucleus, CP: caudate putamen, cp: cerebral penduncle, DSC: dorsal supraoptic decussation of the commissure of Maynert, LGP: lateral globus pallidus, PCom: nucleus of posterior commissure, SNc: substantia nigra pars compacta, SNr: substantia nigra pars reticulata, TRN: thalamic reticular nucleus. FIG. 18C is at 3.5× magnification; FIGS. 18A, 18D, 18E, 18G and 18I are at 10× magnification; FIGS. 18F and 18H are at 25× magnification; FIG. 18B is at 40× magnification.

FIGS. 19A-19H show in vivo transduction of LacZ in the rat hippocampus with VSV-G (19A-19C) and rabies-G (19D-19H) pseudotyped pONY8Z vectors at one month post-injection. In 19A, extensive gene transfer is observed with the VSV-G pseudotyped vector in the subiculum, and to a lesser extent in the CA1 pyramidal cell layer and in the corpus callosum. Faint blue staining represents anterograde transport of β-gal staining of axon fibers projecting to the stratum moleculare (19A and 19B, arrows), and a few fibers projecting to the septum and diagonal band of Broca (19C, arrow). No cell body staining was observed in these regions. These neuronal projections are established from anterograde tracing experiments. FIG. 19D shows strong transduction of CA1 cells with rabies-G compared to VSV-G pseudotyped vectors. Some transduction of CA4 pyramidal cells is also present. FIG. 19E is a higher magnification of the CA1 region depicted in 19D, showing strong staining of apical dendrites and axons of pyramidal neurons. FIG. 19F shows β-gal staining of cells in the subiculum, CA1 pyramidal layer, corpus callosum and cortical fibers in the posterior hippocampus. FIG. 19G shows β-gal staining of CA1 and CA3 pyramidal cells, but not of dentate gyrus in the anterior hippocampus. Cortical fibers are stained, and retrograde labelling of laterodorsal thalamic nucleus is also observed. In FIG. 19H, strong transduction in neuronal nuclei and axons in the lateral hypothalamus and diagonal band of Broca, due to retrograde transport of the rabies-G pseudotyped viral vector is observed. Afferents to the hippocampus from these sites have been previously described. Abbreviations: DG: dentate gyrus; CA1, CA3: hippocampal pyramidal neuronal cell layers; LDVL: vetrolateral aspect of laterodorsal thalamic nucleus; S: subiculum; Se: septum; VDB: vertical limb of the diagonal band of Broca. FIGS. 19A, 19C, 19D and 19F are at 10× magnification; FIG. 19G is at 15× magnification; FIGS. 19B and 19H are at 25× magnification; FIG. 19E is at 50× magnification.

FIGS. 20A-20S show reporter gene expression in the rat spinal cord 3 weeks following intraspinal or intramuscular delivery of pONY8Z lentiviral vectors. FIGS. 20A-20P are micrographs of the ventral horn, showing transduction after intraspinal injections with VSV-G (20A-20G) or rabies-G pseudotyped vector (20H-20P). Strong transduction with β-gal is observed with both types of vectors (20A, 20B, 20H and 20I). FIGS. 20B and 20I are higher magnifications of the area of transduction shown in FIGS. 20A and 20H. Longitudinal sections of the spinal cord show retrogradely fluorogold-labeled motoneurons (20D and 20K) co-expressing β-gal (20C and 20J). Transverse sections stained with anti-β-gal antibodies are shown in FIGS. 20E, 20L and 20Q; the same sections, stained for the neuronal marker NeuN, are shown in FIGS. 20F, 20M and 20R. FIGS. 20G, 20N and 20S are composite confocal images showing neuronal colocalisation of NeuN and β-gal. Retrogradely transduced motoneurons are observed in areas projecting to the site of injection such as brainstem (20O) and layer V of the cerebral cortex (20P) following intraspinal injection of rabies-G pseudotyped pONY8Z vectors. Arrow in FIG. 20H indicates retrogradely transduced commissural motoneurons projecting from the contralateral side to the region of injection, along previously established anatomical connections. The arrowhead in FIG. 20P indicates a transduced layer V corticospinal motoneuron ipsilateral to the injection site. FIGS. 20Q-20S show transverse sections of the spinal cord showing retrograde transport of the viral particles and transduction of spinal cord motoneurons (arrow) after injection of rabies-G pseudotyped pONY8Z vector from the gastrocnemius muscle. FIG. 20Q shows sections stained with anti-β-gal antibodies; FIG. 20R shows the same sections stained for the neuronal marker NeuN. FIG. 20S is a composite confocal image showing colocalisation of NeuN and β-gal. Abbreviations: Vln: vestibular lateral nucleus; Prf: pontine reticular formation. FIGS. 20A and 20H are at 10× magnification; FIGS. 20B-20D, 20I-20K and 20O are at 25× magnification; FIG. 20P is at 50×; FIGS. 20E-20G, 20L-20N and 20Q-20S are at 60× magnification.

FIGS. 21A-21L show the immune response in the rat brain following pONY8Z vector delivery in the rat striatum. Antibodies used to detect components of the immune response in the injected area were as follows: OX1—leucocyte common antigen, OX18—MHC class I, OX42—complement receptor type 3 on microglia and macrophages and OX62—dendritic cells. All animals (including PBS-injected controls) exhibited a minor infiltration of OX42⁺/ED1⁺ activated macrophages/microglia around the needle tract in the cortex and striatum (21C, 21G and 21K). This response declined with time but was still partially evident at 35 days post-injection. Animals injected with VSV-G pseudotyped vectors (21A-21D) exhibited a minor immune response at 7 days post-injection, in addition to the microglial infiltration observed in controls. An infiltration of OX18⁺ MHC class I positive cells in ipsilateral striatum (21B) was observed though neither leucocytes (21A) nor dentritic cells (21D) could be detected at any time after VSV-G pseudotyped vector injection in the brains of these animals. This response had declined by 14 days. Compared to VSV-G pseudotyped vector, a slightly stronger immune response was observed following injection of rabies-G pseudotyped vector. Infiltration of leucocytes (21E and 21I), MHC class I immunopositive cells (21F and 21J), dendritic cells (21H and 21L) and the presence of perivascular cuffing (21E and 21F) can be seen 7 days (21E-21H) after injection, decreasing in levels at 14 days (21I-21L) after injection. FIGS. 21A-21D and 21F-21L are at 25× magnification; FIG. 21E are at 50× magnification.

FIGS. 22A-22E show viral transfer of genes to sensory neurons. The reporter gene β-galactosidase is expressed in the dorsal root (22A-22C) and DRG (22D and 22E) after injection of pONY8Z pseudotyped with rabies-G into the dorsal horn of the spinal cord. The stained sections show immunofluorescence for β-galactosidase 5 weeks after viral injections. Expression of β-gal is detectable in Shwann cells, axons (arrowheads) and DRG neurons (arrows). For immunofluorescence, sections were incubated with rabbit polyclonal anti-β-gal (5Prime3Prime Inc.) at dilution of 1:250. The second antibody used in this experiment was FITC-conjugated anti-rabbit IgG (Jackson Immunoresearch).

FIG. 23 (SEQ ID NO:12) shows the polynucleotide sequence of ERA wild-type.

FIG. 24 (SEQ ID NO: 13) shows the amino acid sequences of ERA wild-type.

FIG. 25 (SEQ ID NO: 14) shows the polynucleotide sequence of ERAdm.

FIG. 26 (SEQ ID NO:15) shows the polynucleotide sequence of CVS rabies virus glycoprotein.

FIGS. 27A-27I show the results of Example 1 and illustrate the transduction efficiency of EIAV-LacZ in the brain following injection into the CSF.

FIGS. 28A-28F show the results of Example 1 and illustrate the expression of the marker gene LacZ in the spinal cord after injection of EIAV-LacZ into the CSF.

FIGS. 29A-29H and 30A-30C show the results of Example 2.

FIGS. 31A-31E show the results of Example 3.

FIGS. 32A-32H show the results of Example 4, using CVS.

FIGS. 33A-33C show, following sub-retinal gene delivery of the pONY8.0 CMVGFP virus, that GFP fluorescence is seen in the optic chiasm (33A), in the axons of the optic tract (33B) and in the cell bodies of the optic tract (33C).

FIG. 34A shows a diagram of a replacement vector comprising the SMN gene.

FIG. 34B shows a diagram of pONY8.7NCSMN.

FIGS. 35A-35D show confocal analysis of SMN immunolabelling following in vitro transduction with Smart2SMN vector pseudotyped with rabies G envelope. FIGS. 35A and 35B show restoration of SMN protein in SMA fibroblast transduced with lentiviral vector-mediated expression of SMN. FIG. 35C shows untransduced cells. FIG. 35D shows β-gal immunostaining in SMA fibroblast transduced with Smart2LacZ. Note the strong staining in the cytoplasm and nucleus in FIGS. 35A and 35B.

FIGS. 36A and 36B show a Western Blot confirming expression of SMN in transduced D17 fibroblasts. D17 cells are transduced with Smart2SMN, SMN-HA and LacZ vectors.

FIGS. 37A and 37B show SMN gene therapy in mild model of SMA. A) Transduction of spinal motor neurons following injection of LentiVector® expressing SMN-HA in Muscle of mice model of type III SMA. B) SMN expression in muscle monitored using antibodies against HA tag.

FIGS. 38A-38C show immune response study in Type III mice after intramuscular injection of Smart2SMN.

FIGS. 39A-39C show SMN gene transfer in mouse model of type I SMA. DRG cells (39A) and spinal motor neurons (39B) were transduced by retrograde transport following intramuscular injection of SMN expressing vectors (39C) control.

### DETAILED DESCRIPTION OF THE INVENTION

The present invention relates to a new use of a vector system.

The vector system can be a non-viral system or a viral system.

Viral vector or viral delivery systems include, but are not limited to, adenoviral vectors, adeno-associated viral (AAV) vectors, herpes viral vectors, retroviral vectors, lentiviral vectors, and baculoviral vectors. Non-viral delivery or non-viral vector systems include lipid mediated transfection, liposomes, immunoliposomes, lipofectin, cationic facial amphiphiles (CFAs) and combinations thereof. In some preferred aspects, the vector system is a viral vector system. In some further preferred aspects, the vector system is a retroviral vector system and, preferably, a lentiviral vector system.

### Retroviruses

The concept of using viral vectors for gene therapy is well known (Verma and Somia (1997) Nature 389:239-242).

There are many retroviruses. For the present application, the term “retrovirus” includes: murine leukaemia virus (MLV), human immunodeficiency virus (HIV), equine infectious anaemia virus (EIAV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV) and all other retroviridiae including lentiviruses.

A detailed list of retroviruses may be found in Coffin et al. (“Retroviruses” 1997 Cold Spring Harbour Laboratory Press Eds: J M Coffin, S M Hughes, H E Varmus pp 758-763).

In a preferred embodiment, the retroviral vector system is derivable from a lentivirus. Lentiviruses also belong to the retrovirus family, but they can infect both dividing and non-dividing cells (Lewis et al. (1992) EMBO J. 3053-3058).

The lentivirus group can be split into “primate” and “non-primate”. Examples of primate lentiviruses include the human immunodeficiency virus (HIV), the causative agent of human acquired immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV).

Details on the genomic structure of some lentiviruses may be found in the art. By way of example, details on HIV and EIAV may be found from the NCBI Genbank database (i.e. Genome Accession Nos. AF033819 and AF033820 respectively).

During the process of infection, a retrovirus initially attaches to a specific cell surface receptor. On entry into the susceptible host cell, the retroviral RNA genome is then copied to DNA by the virally encoded reverse transcriptase which is carried inside the parent virus. This DNA is transported to the host cell nucleus where it subsequently integrates into the host genome. At this stage, it is typically referred to as the provirus. The provirus is stable in the host chromosome during cell division and is transcribed like other cellular genes. The provirus encodes the proteins and other factors required to make more virus, which can leave the cell by a process sometimes called “budding”.

Each retroviral genome comprises genes called gag, pol and env which code for virion proteins and enzymes. These genes are flanked at both ends by regions called long terminal repeats (LTRs). The LTRs are responsible for proviral integration, and transcription. They also serve as enhancer-promoter sequences. In other words, the LTRs can control the expression of the viral genes. Encapsidation of the retroviral RNAs occurs by virtue of a psi sequence located at the 5′ end of the viral genome.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

For the viral genome, the site of transcription initiation is at the boundary between U3 and R in one LTR and the site of poly (A) addition (termination) is at the boundary between R and U5 in the other LTR. U3 contains most of the transcriptional control elements of the provirus, which include the promoter and multiple enhancer sequences responsive to cellular and in some cases, viral transcriptional activator proteins. Some retroviruses have any one or more of the following genes that code for proteins that are involved in the regulation of gene expression: tat, rev, tax and rex.

With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome. The env gene encodes the surface (SU) glycoprotein and the transmembrane (TM) protein of the virion, which form a complex that interacts specifically with cellular receptor proteins. This interaction leads ultimately to infection by fusion of the viral membrane with the cell membrane.

Retroviruses may also contain “additional” genes which code for proteins other than gag, pol and env. Examples of additional genes include in HIV, one or more of vif, vpr, vpx, vpu, tat, rev and nef EIAV has (amongst others) the additional gene S2.

Proteins encoded by additional genes serve various functions, some of which may be duplicative of a function provided by a cellular protein. In EIAV, for example, tat acts as a transcriptional activator of the viral LTR. It binds to a stable, stem-loop RNA secondary structure referred to as TAR. Rev regulates and co-ordinates the expression of viral genes through rev-response elements (RRE). The mechanisms of action of these two proteins are thought to be broadly similar to the analogous mechanisms in the primate viruses. The function of S2 is unknown. In addition, an EIAV protein, Ttm, has been identified that is encoded by the first exon of tat spliced to the env coding sequence at the start of the transmembrane protein.

### Vector Systems

The vector system can be a non-viral system or a viral system.

In some preferred aspects, the vector system is a viral vector system.

In some further preferred aspects, the vector system is a retroviral vector system and, preferably, a lentiviral vector system.

The vector system can be used to transfer an EOI to one or more sites of interest. The transfer can occur in vitro, ex vivo, in vivo, or combinations thereof.

In a highly preferred aspect, the delivery system is a retroviral delivery system which is a lentiviral vector system.

Retroviral vector systems have been proposed as a delivery system for inter alia the transfer of a NOI to one or more sites of interest. The transfer can occur in vitro, ex vivo, in vivo, or combinations thereof. Retroviral vector systems have even been exploited to study various aspects of the retrovirus life cycle, including receptor usage, reverse transcription and RNA packaging (reviewed by Miller, 1992 Curr Top Microbiol Immunol 158:1-24).

As used herein the term “vector system” may also include a vector particle capable of transducing a recipient cell with an NOI.

A vector particle includes the following components: a vector genome, which may contain one or more NOIs, a nucleocapsid encapsidating the nucleic acid, and a membrane surrounding the nucleocapsid.

The term “nucleocapsid” refers to at least the group specific viral core proteins (gag) and the viral polymerase (pol) of a retrovirus genome. These proteins encapsidate the packagable sequences and are themselves further surrounded by a membrane containing an envelope glycoprotein.

Once within the cell, the RNA genome from a retroviral vector particle is reverse transcribed into DNA and integrated into the DNA of the recipient cell.

The term “vector genome” refers both to the RNA construct present in the retroviral vector particle and the integrated DNA construct. The term also embraces a separate or isolated DNA construct capable of encoding such an RNA genome. A retroviral or lentiviral genome should comprise at least one component part derivable from a retrovirus or a lentivirus. The term “derivable” is used in its normal sense as meaning a nucleotide sequence or a part thereof which need not necessarily be obtained from a virus such as a lentivirus but instead could be derived therefrom. By way of example, the sequence may be prepared synthetically or by use of recombinant DNA techniques. Preferably the genome comprises a psi region (or an analogous component which is capable of causing encapsidation).

The viral vector genome is preferably “replication defective” by which we mean that the genome does not comprise sufficient genetic information alone to enable independent replication to produce infectious viral particles within the recipient cell. In a preferred embodiment, the genome lacks a functional env, gag or pol gene. If a highly preferred embodiment the genome lacks env, gag and pol genes.

The viral vector genome may comprise some or all of the long terminal repeats (LTRs). Preferably the genome comprises at least part of the LTRs or an analogous sequence which is capable of mediating proviral integration, and transcription. The sequence may also comprise or act as an enhancer-promoter sequence.

It is known that the separate expression of the components required to produce a retroviral vector particle on separate DNA sequences cointroduced into the same cell will yield retroviral particles carrying defective retroviral genomes that carry therapeutic genes (e.g. Reviewed by Miller 1992). This cell is referred to as the producer cell (see below).

There are two common procedures for generating producer cells. In one, the sequences encoding retroviral Gag, Pol and Env proteins are introduced into the cell and stably integrated into the cell genome; a stable cell line is produced which is referred to as the packaging cell line. The packaging cell line produces the proteins required for packaging retroviral RNA but it cannot bring about encapsidation due to the lack of a psi region. However, when a vector genome (having a psi region) is introduced into the packaging cell line, the helper proteins can package the psi-positive recombinant vector RNA to produce the recombinant virus stock. This can be used to transduce the NOI into recipient cells. The recombinant virus whose genome lacks all genes required to make viral proteins can infect only once and cannot propagate. Hence, the NOI is introduced into the host cell genome without the generation of potentially harmful retrovirus. A summary of the available packaging lines is presented in “Retroviruses” (1997 Cold Spring Harbour Laboratory Press Eds: J M Coffin, S M Hughes, H E Varmus pp 449).

The present invention also provides a packaging cell line comprising a viral vector genome which is capable of producing a vector system useful in the first aspect of the invention. For example, the packaging cell line may be transduced with a viral vector system comprising the genome or transfected with a plasmid carrying a DNA construct capable of encoding the RNA genome. The present invention also provides a kit for producing a retroviral vector system useful in the first aspect of the invention which comprises a packaging cell and a retroviral vector genome.

The second approach is to introduce the three different DNA sequences that are required to produce a retroviral vector particle i.e. the env coding sequences, the gag-pol coding sequence and the defective retroviral genome containing one or more NOIs into the cell at the same time by transient transfection and the procedure is referred to as transient triple transfection (Landau & Littman 1992; Pear et al. 1993). The triple transfection procedure has been optimised (Soneoka et al. 1995; Finer et al. 1994). WO 94/29438 describes the production of producer cells in vitro using this multiple DNA transient transfection method. WO 97/27310 describes a set of DNA sequences for creating retroviral producer cells either in vivo or in vitro for re-implantation.

The components of the viral system which are required to complement the vector genome may be present on one or more “producer plasmids” for transfecting into cells.

The present invention also provides a kit for producing a retroviral vector system useful in the first aspect of the invention, comprising:
(i) a viral vector genome which is incapable of encoding one or more proteins which are required to produce a vector particle;
(ii) one or more producer plasmid(s) capable of encoding the protein which is not encoded by (i); and optionally
(iii) a cell suitable for conversion into a producer cell.

In a preferred embodiment, the viral vector genome is incapable of encoding the proteins gag, pol and env. Preferably the kit comprises one or more producer plasmids encoding env, gag and pol, for example, one producer plasmid encoding env and one encoding gag-pol. Preferably the gag-pol sequence is codon optimised for use in the particular producer cell (see below).

The present invention also provides a producer cell expressing the vector genome and the producer plasmid(s) capable of producing a retroviral vector system useful in the present invention.

Preferably the retroviral vector system used in the first aspect of the present invention is a self-inactivating (SIN) vector system.

By way of example, self-inactivating retroviral vector systems have been constructed by deleting the transcriptional enhancers or the enhancers and promoter in the U3 region of the 3′ LTR. After a round of vector reverse transcription and integration, these changes are copied into both the 5′ and the 3′ LTRs producing a transcriptionally inactive provirus. However, any promoter(s) internal to the LTRs in such vectors will still be transcriptionally active. This strategy has been employed to eliminate effects of the enhancers and promoters in the viral LTRs on transcription from internally placed genes. Such effects include increased transcription or suppression of transcription. This strategy can also be used to eliminate downstream transcription from the 3′ LTR into genomic DNA. This is of particular concern in human gene therapy where it may be important to prevent the adventitious activation of an endogenous oncogene.

Preferably a recombinase assisted mechanism is used which facilitates the production of high titre regulated lentiviral vectors from the producer cells of the present invention.

As used herein, the term “recombinase assisted system” includes but is not limited to a system using the Cre recombinase/loxP recognition sites of bacteriophage P1 or the site-specific FLP recombinase of S. cerevisiae which catalyses recombination events between 34 bp FLP recognition targets (FRTs).

The site-specific FLP recombinase of S. cerevisiae which catalyses recombination events between 34 bp FLP recognition targets (FRTs) has been configured into DNA constructs in order to generate high level producer cell lines using recombinase-assisted recombination events (Karreman et al. (1996) NAR 24:1616-1624). A similar system has been developed using the Cre recombinase/loxP recognition sites of bacteriophage P1 (see PCT/GB00/03837; Vanin et al. (1997) J. Virol 71:7820-7826). This was configured into a lentiviral genome such that high titre lentiviral producer cell lines were generated.

By using producer/packaging cell lines, it is possible to propagate and isolate quantities of retroviral vector particles (e.g. to prepare suitable titres of the retroviral vector particles) for subsequent transduction of, for example, a site of interest (such as adult brain tissue). Producer cell lines are usually better for large scale production of vector particles.

Transient transfection has numerous advantages over the packaging cell method. In this regard, transient transfection avoids the longer time required to generate stable vector-producing cell lines and is used if the vector genome or retroviral packaging components are toxic to cells. If the vector genome encodes toxic genes or genes that interfere with the replication of the host cell, such as inhibitors of the cell cycle or genes that induce apoptosis, it may be difficult to generate stable vector-producing cell lines, but transient transfection can be used to produce the vector before the cells die. Also, cell lines have been developed using transient infection that produce vector titre levels that are comparable to the levels obtained from stable vector-producing cell lines (Pear et al. 1993, PNAS 90:8392-8396).

Producer cells/packaging cells can be of any suitable cell type. Producer cells are generally mammalian cells but can be, for example, insect cells.

As used herein, the term “producer cell” or “vector producing cell” refers to a cell which contains all the elements necessary for production of retroviral vector particles.

Preferably, the producer cell is obtainable from a stable producer cell line.

Preferably, the producer cell is obtainable from a derived stable producer cell line.

Preferably, the producer cell is obtainable from a derived producer cell line.

As used herein, the term “derived producer cell line” is a transduced producer cell line which has been screened and selected for high expression of a marker gene. Such cell lines support high level expression from the retroviral genome. The term “derived producer cell line” is used interchangeably with the term “derived stable producer cell line” and the term “stable producer cell line.

Preferably the derived producer cell line includes but is not limited to a retroviral and/or a lentiviral producer cell.

Preferably the derived producer cell line is an HIV or EIAV producer cell line, more preferably an EIAV producer cell line.

Preferably the envelope protein sequences, and nucleocapsid sequences are all stably integrated in the producer and/or packaging cell. However, one or more of these sequences could also exist in episomal form and gene expression could occur from the episome.

As used herein, the term “packaging cell” refers to a cell which contains those elements necessary for production of infectious recombinant virus which are lacking in the RNA genome. Typically, such packaging cells contain one or more producer plasmids which are capable of expressing viral structural proteins (such as gag-pol and env, which may be codon optimised) but they do not contain a packaging signal.

The term “packaging signal” which is referred to interchangeably as “packaging sequence” or “psi” is used in reference to the non-coding, cis-acting sequence required for encapsidation of retroviral RNA strands during viral particle formation. In HIV-1, this sequence has been mapped to loci extending from upstream of the major splice donor site (SD) to at least the gag start codon.

Packaging cell lines may be readily prepared (see also WO 92/05266), and utilised to create producer cell lines for the production of retroviral vector particles. As already mentioned, a summary of the available packaging lines is presented in “Retroviruses” (as above).

Also as discussed above, simple packaging cell lines, comprising a provirus in which the packaging signal has been deleted, have been found to lead to the rapid production of undesirable replication competent viruses through recombination. In order to improve safety, second generation cell lines have been produced wherein the 3′LTR of the provirus is deleted. In such cells, two recombinations would be necessary to produce a wild type virus. A further improvement involves the introduction of the gag-pol genes and the env gene on separate constructs so-called third generation packaging cell lines. These constructs are introduced sequentially to prevent recombination during transfection.

Preferably, the packaging cell lines are second generation packaging cell lines.

Preferably, the packaging cell lines are third generation packaging cell lines.

In these split-construct, third generation cell lines, a further reduction in recombination may be achieved by changing the codons. This technique, based on the redundancy of the genetic code, aims to reduce homology between the separate constructs, for example between the regions of overlap in the gag-pol and env open reading frames.

The packaging cell lines are useful for providing the gene products necessary to encapsidate and provide a membrane protein for a high titre vector particle production. The packaging cell may be a cell cultured in vitro such as a tissue culture cell line. Suitable cell lines include but are not limited to mammalian cells such as murine fibroblast derived cell lines or human cell lines. Preferably the packaging cell line is a human cell line, such as for example: HEK293, 293-T, TE671, HT1080.

Alternatively, the packaging cell may be a cell derived from the individual to be treated such as a monocyte, macrophage, blood cell or fibroblast. The cell may be isolated from an individual and the packaging and vector components administered ex vivo followed by re-administration of the autologous packaging cells.

It is highly desirable to use high-titre virus preparations in both experimental and practical applications. Techniques for increasing viral titre include using a psi plus packaging signal as discussed above and concentration of viral stocks.

As used herein, the term “high titre” means an effective amount of a retroviral vector or particle which is capable of transducing a target site such as a cell.

As used herein, the term “effective amount” means an amount of a regulated retroviral or lentiviral vector or vector particle which is sufficient to induce expression of the NOIs at a target site.

A high-titre viral preparation for a producer/packaging cell is usually of the order of 10⁵ to 10⁷ t.u. per ml. (The titre is expressed in transducing units per ml (t.u./ml) as titred on a standard D17 cell line). For transduction in tissues such as the brain, it is necessary to use very small volumes, so the viral preparation is concentrated by ultracentrifugation. The resulting preparation should have at least 10⁸ t.u./ml, preferably from 10⁸ to 10⁹ t.u./ml, more preferably at least 10⁹ t.u./ml.

The expression products encoded by the NOIs may be proteins which are secreted from the cell. Alternatively the NOI expression products are not secreted and are active within the cell. For some applications, it is preferred for the NOI expression product to demonstrate a bystander effect or a distant bystander effect; that is the production of the expression product in one cell leading to the modulation of additional, related cells, either neighbouring or distant (e.g. metastatic), which possess a common phenotype.

The presence of a sequence termed the central polypurine tract (cPPT) may improve the efficiency of gene delivery to non-dividing cells (see WO 00/31200). This cis-acting element is located, for example, in the EIAV polymerase coding region element. Preferably the genome of the vector system used in the present invention comprises a cPPT sequence.

In addition, or in the alternative, the viral genome may comprise a post-translational regulatory element and/or a translational enhancer.

The NOIs may be operatively linked to one or more promoter/enhancer elements. Transcription of one or more NOI may be under the control of viral LTRs or alternatively promoter-enhancer elements can be engineered in with the transgene. Preferably the promoter is a strong promoter such as CMV. The promoter may be a regulated promoter. The promoter may be tissue-specific. In a preferred embodiment the promoter is glial cell-specific. In another preferred embodiment the promoter is neuron-specific.

### Minimal Systems

It has been demonstrated that a primate lentivirus minimal system can be constructed which requires none of the HIV/SIV additional genes vif, vpr, vpx, vpu, tat, rev and nef for either vector production or for transduction of dividing and non-dividing cells. It has also been demonstrated that an EIAV minimal vector system can be constructed which does not require S2 for either vector production or for transduction of dividing and non-dividing cells. The deletion of additional genes is highly advantageous. Firstly, it permits vectors to be produced without the genes associated with disease in lentiviral (e.g. HIV) infections. In particular, tat is associated with disease. Secondly, the deletion of additional genes permits the vector to package more heterologous DNA. Thirdly, genes whose function is unknown, such as S2, may be omitted, thus reducing the risk of causing undesired effects. Examples of minimal lentiviral vectors are disclosed in WO-A-99/32646 and in WO-A-98/17815.

Thus, preferably, the delivery system used in the invention is devoid of at least tat and S2 (if it is an EIAV vector system), and possibly also vif, vpr, vpx, vpu and nef. More preferably, the systems of the present invention are also devoid of rev. Rev was previously thought to be essential in some retroviral genomes for efficient virus production. For example, in the case of HIV, it was thought that rev and RRE sequence should be included. However, it has been found that the requirement for rev and RRE can be reduced or eliminated by codon optimisation (see below) or by replacement with other functional equivalent systems such as the MPMV system. As expression of the codon optimised gag-pol is REV independent, RRE can be removed from the gag-pol expression cassette, thus removing any potential for recombination with any RRE contained on the vector genome.

In a preferred embodiment the viral genome of the first aspect of the invention lacks the Rev response element (RRE).

In a preferred embodiment, the system used in the present invention is based on a so-called “minimal” system in which some or all of the additional genes have been removed.

### Codon Optimisation

Codon optimisation has previously been described in WO99/41397. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

Many viruses, including HIV and other lentiviruses, use a large number of rare codons and by changing these to correspond to commonly used mammalian codons, increased expression of the packaging components in mammalian producer cells can be achieved. Codon usage tables are known in the art for mammalian cells, as well as for a variety of other organisms.

Codon optimisation has a number of other advantages. By virtue of alterations in their sequences, the nucleotide sequences encoding the packaging components of the viral particles required for assembly of viral particles in the producer cells/packaging cells have RNA instability sequences (INS) eliminated from them. At the same time, the amino acid sequence coding sequence for the packaging components is retained so that the viral components encoded by the sequences remain the same, or at least sufficiently similar that the function of the packaging components is not compromised. Codon optimisation also overcomes the Rev/RRE requirement for export, rendering optimised sequences Rev independent. Codon optimisation also reduces homologous recombination between different constructs within the vector system (for example between the regions of overlap in the gag-pol and env open reading frames). The overall effect of codon optimisation is therefore a notable increase in viral titre and improved safety.

In one embodiment only codons relating to INS are codon optimised. However, in a much more preferred and practical embodiment, the sequences are codon optimised in their entirety, with the exception of the sequence encompassing the frameshift site.

The gag-pol gene comprises two overlapping reading frames encoding the gag-pol proteins. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames is preferably not codon optimised. Retaining this fragment will enable more efficient expression of the gag-pol proteins.

For EIAV the beginning of the overlap has been taken to be nt 1262 (where nucleotide 1 is the A of the gag ATG). The end of the overlap is at 1461 bp. In order to ensure that the frameshift site and the gag-pol overlap are preserved, the wild type sequence has been retained from nt 1156 to 1465.

Derivations from optimal codon usage may be made, for example, in order to accommodate convenient restriction sites, and conservative amino acid changes may be introduced into the gag-pol proteins.

In a highly preferred embodiment, codon optimisation was based on lightly expressed mammalian genes. The third and sometimes the second and third base may be changed.

Due to the degenerate nature of the Genetic Code, it will be appreciated that numerous gag-pol sequences can be achieved by a skilled worker. Also, there are many retroviral variants described which can be used as a starting point for generating a codon optimised gag-pol sequence. Lentiviral genomes can be quite variable. For example there are many quasi-species of HIV-1 which are still functional. This is also the case for EIAV. These variants may be used to enhance particular parts of the transduction process. Examples of HIV-1 variants may be found in the HIV databases maintained by Los Alamos National Laboratory. Details of EIAV clones may be found at the NCBI database maintained by the National Institutes of Health.

The strategy for codon optimised gag-pol sequences can be used in relation to any retrovirus. This would apply to all lentiviruses, including EIAV, FIV, BIV, CAEV, VMR, SIV, HIV-1 and HIV-2. In addition this method could be used to increase expression of genes from HTLV-1, HTLV-2, HFV, HSRV and human endogenous retroviruses (HERV), MLV and other retroviruses.

Codon optimisation can render gag-pol expression Rev independent. In order to enable the use of anti-rev or RRE factors in the retroviral vector, however, it would be necessary to render the viral vector generation system totally Rev/RRE independent. Thus, the genome also needs to be modified. This is achieved by optimising vector genome components. Advantageously, these modifications also lead to the production of a safer system absent of all additional proteins both in the producer and in the transduced cell.

As described above, the packaging components for a retroviral vector include expression products of gag, pol and env genes. In addition, efficient packaging depends on a short sequence of 4 stem loops followed by a partial sequence from gag and env (the “packaging signal”). Thus, inclusion of a deleted gag sequence in the retroviral vector genome (in addition to the full gag sequence on the packaging construct) will optimise vector titre. To date efficient packaging has been reported to require from 255 to 360 nucleotides of gag in vectors that still retain env sequences, or about 40 nucleotides of gag in a particular combination of splice donor mutation, gag and env deletions. It has surprisingly been found that a deletion of all but the N-terminal 360 or so nucleotides in gag leads to an increase in vector titre. Thus, preferably, the retroviral vector genome includes a gag sequence which comprises one or more deletions, more preferably the gag sequence comprises about 360 nucleotides derivable from the N-terminus.

### Pseudotyping

In the design of retroviral vector systems it is desirable to engineer particles with different target cell specificities to the native virus, to enable the delivery of genetic material to an expanded or altered range of cell types. One manner in which to achieve this is by engineering the virus envelope protein to alter its specificity. Another approach is to introduce a heterologous envelope protein into the vector particle to replace or add to the native envelope protein of the virus.

The term pseudotyping means incorporating in at least a part of, or substituting a part of, or replacing all of, an env gene of a viral genome with a heterologous env gene, for example an env gene from another virus. Pseudotyping is not a new phenomenon and examples may be found in WO 99/61639, WO-A-98/05759, WO-A-98/05754, WO-A-97/17457, WO-A-96/09400, WO-A-91/00047 and Mebatsion et al. 1997 Cell 90, 841-847.

Pseudotyping can improve retroviral vector stability and transduction efficiency. A pseudotype of murine leukemia virus packaged with lymphocytic choriomeningitis virus (LCMV) has been described (Miletic et al. (1999) J. Virol. 73:6114-6116) and shown to be stable during ultracentrifugation and capable of infecting several cell lines from different species.

In the present invention the vector system may be pseudotyped with at least a part of a rabies G envelope protein, or a mutant, variant, homologue or fragment thereof.

Thus, the retroviral delivery system used in the first aspect of the invention comprises a first nucleotide sequence coding for at least a part of an envelope protein; and one or more other nucleotide sequences derivable from a retrovirus that ensure transduction by the retroviral delivery system; wherein the first nucleotide sequence is heterologous with respect to at least one of the other nucleotide sequences; and wherein the first nucleotide sequence codes for at least a part of a rabies G envelope protein or a mutant, variant, homologue or fragment thereof.

There is thus provided the use of a retroviral delivery system comprising a heterologous env region, wherein the heterologous env region comprises at least a part of a rabies G protein or a mutant, variant, homologue or fragment thereof or at least a part of a CVS protein or a mutant, variant, homologue or fragment thereof.

The heterologous env region may be encoded by a gene which is present on a producer plasmid. The producer plasmid may be present as part of a kit for the production of retroviral vector particles suitable for use in the first aspect of the invention.

### Rabies G Protein

In the present invention the vector system may be pseudotyped with at least a part of a rabies G protein or a mutant, variant, homologue or fragment thereof.

Teachings on the rabies G protein, as well as mutants thereof, may be found in WO 99/61639 and well as Rose et al., 1982 J. Virol. 43: 361-364, Hanham et al., 1993 J. Virol., 67, 530-542, Tuffereau et al., 1998 J. Virol., 72, 1085-1091, Kucera et al., 1985 J. Virol 55, 158-162, Dietzschold et al., 1983 PNAS 80, 70-74, Seif et al., 1985 J. Virol., 53, 926-934, Coulon et al., 1998 J. Virol., 72, 273-278, Tuffereau et al., 1998 J. Virol., 72, 1085-10910, Burger et al., 1991 J. Gen. Virol. 72. 359-367, Gaudin et al., 1995 J Virol 69, 5528-5534, Benmansour et al., 1991 J Virol 65, 4198-4203, Luo et al., 1998 Microbiol Immunol 42, 187-193, Coll 1997 Arch Virol 142, 2089-2097, Luo et al., 1997 Virus Res 51, 35-41, Luo et al., 1998 Microbiol Immunol 42, 187-193, Coll 1995 Arch Virol 140, 827-851, Tuchiya et al., 1992 Virus Res 25, 1-13, Morimoto et al., 1992 Virology 189, 203-216, Gaudin et al., 1992 Virology 187, 627-632, Whitt et al., 1991 Virology 185, 681-688, Dietzschold et al., 1978 J Gen Virol 40, 131-139, Dietzschold et al., 1978 Dev Biol Stand 40, 45-55, Dietzschold et al., 1977 J Virol 23, 286-293, and Otvos et al., 1994 Biochim Biophys Acta 1224, 68-76. A rabies G protein is also described in EP-A-0445625.

The present invention provides a rabies G protein having the amino acid sequence shown in SEQ ID NO.3. The present invention also provides a nucleotide sequence capable of encoding such a rabies G protein. Preferably the nucleotide sequence comprises the sequence shown in SEQ ID NO. 4.

These sequences differ from the Genbank sequence as shown below:

(SEQ ID NO: 7)

I Y T I L D K L

(SEQ ID NO: 6)

| | |
|---|---|
| Genbank | ATT TAC ACG ATA CTA GAC AAG CTT |
| sequence | |

(SEQ ID NO: 9)

I Y T I P D K L

(SEQ ID NO: 8)

| | |
|---|---|
| Present | ATT TAC ACG ATC CCA GAC AAG CTT |
| Invention | |

In a preferred embodiment, the vector system of the present invention is or comprises at least a part of a rabies G protein having the amino acid sequence shown in SEQ ID NO.3.

The use of rabies G protein provides vectors which, in vivo, preferentially transduce targeted cells which rabies virus preferentially infects. This includes in particular neuronal target cells in vivo. For a neuron-targeted vector, rabies G from a pathogenic strain of rabies such as ERA may be particularly effective. On the other hand rabies G protein confers a wider target cell range in vitro including nearly all mammalian and avian cell types tested (Seganti et al., 1990 Arch Virol. 34, 155-163; Fields et al., 1996 Fields Virology, Third Edition, vol. 2, Lippincott-Raven Publishers, Philadelphia, N.Y.).

The tropism of the pseudotyped vector particles may be modified by the use of a mutant rabies G which is modified in the extracellular domain. Rabies G protein has the advantage of being mutatable to restrict target cell range. The uptake of rabies virus by target cells in vivo is thought to be mediated by the acetylcholine receptor (AchR) but there may be other receptors to which in binds in vivo (Hanham et al., 1993 J. Virol., 67, 530-542; Tuffereau et al., 1998 J. Virol., 72, 1085-1091). It is thought that multiple receptors are used in the nervous system for viral entry, including NCAM (Thoulouze et al., (1998) J. Virol 72(9):7181-90) and p75 Neurotrophin receptor (Tuffereau C et al. (1998) EMBO J 17(24) 7250-9).

The effects of mutations in antigenic site III of the rabies G protein on virus tropism have been investigated, this region it is reported is not thought to be involved in the binding of the virus to the acetylcholine receptor (Kucera et al., 1985 J. Virol 55, 158-162; Dietzschold et al., 1983 Proc Natl Acad Sci 80, 70-74; Seif et al., 1985 J. Virol., 53, 926-934; Coulon et al., 1998 J. Virol., 72, 273-278; Tuffereau et al., 1998 J. Virol., 72, 1085-10910). For example it has been reported that a mutation of the arginine at amino acid 333 in the mature protein to glutamine (i.e. ERAsm) can be used to restrict viral entry to olfactory and peripheral neurons in vivo while reducing propagation to the central nervous system. It has also been reported that these viruses were able to penetrate motor neurons and sensory neurons as efficiently as the wild type virus, yet transneuronal transfer did not occur (Coulon et al., 1989, J. Virol. 63, 3550-3554). Viruses in which amino acid 330 has been mutated are further attenuated (i.e. ERAdm), were reported as being unable to infect either motor neurons or sensory neurons after intramuscular injection (Coulon et al., 1998 J. Virol., 72, 273-278).

Alternatively or additionally, rabies G proteins from laboratory passaged strains of rabies may be used. These can be screened for alterations in tropism. Such strains include the following:

TABLE 1

| Genbank accession number | Rabies Strain |
|---|---|
| J02293 | ERA |
| U52947 | COSRV |
| U27214 | NY 516 |
| U27215 | NY771 |
| U27216 | FLA125 |
| U52946 | SHBRV |
| M32751 | HEP-Flury |

By way of example, the ERA strain is a pathogenic strain of rabies and the rabies G protein from this strain can be used for transduction of neuronal cells. The sequence of rabies G from the ERA strains is in the GenBank database (Accession number J02293). This protein has a signal peptide of 19 amino acids and the mature protein begins at the lysine residue 20 amino acids from the translation initiation methionine. The HEP-Flury strain contains the mutation from arginine to glutamine at amino acid position 333 in the mature protein which correlates with reduced pathogenicity and which can be used to restrict the tropism of the viral envelope.

WO 99/61639 discloses the nucleic and amino acid sequences for a rabies virus strain ERA (Genbank locus RAVGPLS, Accession no. M38452).

In the present invention the vector system may be pseudotyped with at least part of a protein from the Challenge Virus Standard (CVS) strain of rabies virus, and in particular the CVS glycoprotein G, or a mutant, variant, homologue or fragment thereof. The cDNA for CVS rabiesvirus G is different in nucleotide sequence from ERA rabiesvirus G; teachings on CVS can be found in U.S. Pat. No. 5,348,741. ATCC deposit No. 40280, designated pKB3-JE-13, may conveniently be used in the present invention.

It will also be appreciated that CVS glycoproteins from laboratory passaged strains of CVS may be used. These can be screened for alterations in tropism.

It will further be appreciated that the instant invention encompasses vectors encoding equivalents of rabies G glycoprotein.

Accession information is provided merely as convenience to those of skill in the art, and are not an admission that deposits are required under 35 U.S.C. §112. The viral strains are incorporated herein by reference and are controlling in the event of any conflict with the description herein.

### Mutants, Variants, Homologues and Fragments

The vector system is or comprises at least part of a wild-type rabies G protein or a mutant, variant, homologue or fragment thereof.

The term “wild type” is used to mean a polypeptide having a primary amino acid sequence which is identical with the native protein (i.e., the viral protein).

The term “mutant” is used to mean a polypeptide having a primary amino acid sequence which differs from the wild type sequence by one or more amino acid additions, substitutions or deletions. A mutant may arise naturally, or may be created artificially (for example by site-directed mutagenesis). Preferably the mutant has at least 90% sequence identity with the wild type sequence. Preferably the mutant has 20 mutations or less over the whole wild-type sequence. More preferably the mutant has 10 mutations or less, most preferably 5 mutations or less over the whole wild-type sequence.

The term “variant” is used to mean a naturally occurring polypeptide which differs from a wild-type sequence. A variant may be found within the same viral strain (i.e. if there is more than one isoform of the protein) or may be found within a different strains. Preferably the variant has at least 90% sequence identity with the wild type sequence. Preferably the variant has 20 mutations or less over the whole wild-type sequence. More preferably the variant has 10 mutations or less, most preferably 5 mutations or less over the whole wild-type sequence.

Here, the term “homologue” means an entity having a certain homology with the wild type amino acid sequence and the wild type nucleotide sequence. Here, the term “homology” can be equated with “identity”.

In the present context, a homologous sequence is taken to include an amino acid sequence which may be at least 75, 85 or 90% identical, preferably at least 95 or 98% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, a homologous sequence is taken to include a nucleotide sequence which may be at least 75, 85 or 90% identical, preferably at least 95 or 98% identical to the subject sequence. Typically, the homologues will comprise the same sequences that code for the active sites etc. as the subject sequence. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

Percent homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However, for some applications, it is preferred to use the GCG Bestfit program. A new tool, called BLAST 2 Sequences is also available for comparing protein and nucleotide sequence (see FEMS Microbiol Lett 1999 174(2): 247-50; FEMS Microbiol Lett 1999 177(1): 187-8).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

The sequences may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to Table 2. Amino acids in the same block in the second column and preferably in the same line in the third column may be substituted for each other:

TABLE 2

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The present invention also encompasses homologous substitution (substitution and replacement are both used herein to mean the interchange of an existing amino acid residue, with an alternative residue) may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as 0), pyriylalanine, thienylalanine, naphthylalanine and phenylglycine.

Replacements may also be made by unnatural amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, β-alanine*, L-α-amino butyric acid*, L-γ-amino butyric acid*, L-α-amino isobutyric acid*, L-ε-amino caproic acid^{#}, 7-amino heptanoic acid*, L-methionine sulfone^{#}*, L-norleucine*, L-norvaline*, p-nitro-L-phenylalanine*, L-hydroxyproline^{#}, L-thioproline*, methyl derivatives of phenylalanine (Phe) such as 4-methyl-Phe*, pentamethyl-Phe*, L-Phe (4-amino)^{#}, L-Tyr (methyl)*, L-Phe (4-isopropyl)*, L-Tic (1,2,3,4-tetrahydroisoquinoline-3-carboxyl acid)*, L-diaminopropionic acid^{#} and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

The term “fragment” indicates that the polypeptide comprises a fraction of the wild-type amino acid sequence. It may comprise one or more large contiguous sections of sequence or a plurality of small sections. The polypeptide may also comprise other elements of sequence, for example, it may be a fusion protein with another protein. Preferably the polypeptide comprises at least 50%, more preferably at least 65%, most preferably at least 80% of the wild-type sequence.

With respect to function, the mutant, variant, homologue or fragment should be capable of transducing at least part of the brain, a motor neuron or cerebrospinal fluid (CSF when used to pseudotype an appropriate vector.

The mutant, variant, homologue or fragment should alternatively or in addition, be capable of conferring the capacity for retrograde transport on the vector system.

The vector delivery system used in the present invention may comprise nucleotide sequences that can hybridise to the nucleotide sequence presented herein (including complementary sequences of those presented herein). In a preferred aspect, the present invention covers nucleotide sequences that can hybridise to the nucleotide sequence of the present invention under stringent conditions (e.g. 65° C. and 0.1 SSC) to the nucleotide sequence presented herein (including complementary sequences of those presented herein).

A potential advantage of using the rabies glycoprotein is the detailed knowledge of its toxicity to humans and other animals due to the extensive use of rabies vaccines. In particular, phase 1 clinical trials have been reported on the use of rabies glycoprotein expressed from canarypox recombinant virus as a human vaccine (Fries et al., 1996 Vaccine 14, 428-434); these studies concluded that the vaccine was safe for use in humans.

### TH Positive Neurons

As used herein, the term “TH positive neurons” are neural cells which are capable of producing tyrosine hydroxylase (TH). The production of tyrosine hydroxylase can be determined by known techniques which measure production of tyrosine hydroxylase mRNA (polymerase chain reaction (PCR), Northern blotting) or protein (immunolabelling, radiolabelling, ELISA-based techniques). Also, the production of metabolites may be measured by known techniques including HPLC with electrochemical detection. TH is expressed by dopaminergic neurons, noradrenergic neurons and adrenal cells.

Mesencephalic, catecholaminergic TH positive cells are capable of producing dopamine. The production of dopamine and noradrenaline is summarised below:

Tyrosine-1→L-DOPA-2→Dopamine-3→noradrenaline

1=Tyrosine hydroxylase

2=DOPA decarboxylase

3=Dopamine-betahydroxylase

Noradrenaergic neurones express all three enzymes, whereas dopaminergic neurones express Tyrosine hydroxylase and DOPA decarboxylase, but lack Dopamine-betahydroxylase.

Tyrosine hydroxylase is the rate-limiting enzyme in the biochemical pathway for dopamine production and is commonly used in the art as a marker for dopaminergic neurons. Dopaminergic neurons may be distinguished from noradrenergic neurones by the absence of Dopamine betahydroxylase within the cells.

TH positive cells may be found in or isolated from dopaminergic neural tissue. Dopaminergic neural tissue is derivable from regions of the CNS which, in the mature state, contains significant numbers of dopaminergic cell bodies. Dopaminergic neural tissue is found in regions of the retina, olfactory bulb, hypothalamus, dorsal motor nucleus, nucleus tractus solitarious, periaqueductal gray matter, ventral tegmenum, and substantia nigra.

### Entities/Nucleotides of Interest

In a broad aspect, the present invention relates to a vector system that is capable of transporting an entity of interest (EOI). The EOI can be a chemical compound, a biological compound or a combination thereof. For example, the EOI can be protein (e.g. a growth factor), a nucleotide sequence, an organic and/or inorganic pharmaceutical (e.g. an analgesic, anti-inflammatory, hormone or lipid), or a combination thereof. Preferably the EOI is one or more NOIs (nucleotide sequences of interest), wherein said NOIs can be delivered to a target cell in vivo or in vitro.

If the vector system of the present invention is a viral vector system, then it is possible to manipulate the viral genome so that viral genes are replaced or supplemented with one or more NOIs which may be heterologous NOIs.

The term “heterologous” refers to a nucleic acid or protein sequence linked to a nucleic acid or protein sequence to which it is not naturally linked.

In the present invention, the term NOI includes any suitable nucleotide sequence, which need not necessarily be a complete naturally occurring DNA or RNA sequence. Thus, the NOI can be, for example, a synthetic RNA/DNA sequence, a recombinant RNA/DNA sequence (i.e. prepared by use of recombinant DNA techniques), a cDNA sequence or a partial genomic DNA sequence, including combinations thereof. The sequence need not be a coding region. If it is a coding region, it need not be an entire coding region. In addition, the RNA/DNA sequence can be in a sense orientation or in an anti-sense orientation. Preferably, it is in a sense orientation. Preferably, the sequence is, comprises, or is transcribed from cDNA.

The retroviral vector genome may generally comprise LTRs at the 5′ and 3′ ends, suitable insertion sites for inserting one or more NOI(s), and/or a packaging signal to enable the genome to be packaged into a vector particle in a producer cell. There may even be suitable primer binding sites and integration sites to allow reverse transcription of the vector RNA to DNA, and integration of the proviral DNA into the target cell genome. In a preferred embodiment, the retroviral vector particle has a reverse transcription system (compatible reverse transcription and primer binding sites) and an integration system (compatible integrase and integration sites).

The NOI may encode a protein of interest (“POI”). In this way, the vector delivery system could be used to examine the effect of expression of a foreign gene on the target cell (such as a TH positive neuron). For example, the retroviral delivery system could be used to screen a cDNA library for a particular effect on the brain, motor neuron or CSF.

For example, one could identify new survival/neuroprotective factors for dopaminergic neurons, which would enable transfected TH+ cells to persist in the presence of an apoptosis-inducing factor.

In accordance with the present invention, suitable NOIs include those that are of therapeutic and/or diagnostic application such as, but not limited to: sequences encoding cytokines, chemokines, hormones, antibodies, anti-oxidant molecules, engineered immunoglobulin-like molecules, a single chain antibody, fusion proteins, enzymes, immune co-stimulatory molecules, immunomodulatory molecules, anti-sense RNA, a transdominant negative mutant of a target protein, a toxin, a conditional toxin, an antigen, a tumour suppresser protein and growth factors, membrane proteins, vasoactive proteins and peptides, anti-viral proteins and ribozymes, and derivatives thereof (such as with an associated reporter group). The NOIs may also encode pro-drug activating enzymes.

The expression products encoded by the NOIs may be proteins which are secreted from the cell. Alternatively the NOI expression products are not secreted and are active within the cell. In either event, it is preferred for the NOI expression product to demonstrate a bystander effect or a distant bystander effect; that is the production of the expression product in one cell leading to the killing of additional, related cells, either neighbouring or distant (e.g. metastatic), which possess a common phenotype.

The NOI or its expression product may act to modulate the biological activity of a compound or a pathway. As used herein the term “modulate” includes for example enhancing or inhibiting biological activity. Such modulation may be direct (e.g. including cleavage of, or competitive binding of another substance to a protein) or indirect (e.g. by blocking the initial production of a protein).

The NOI may be capable of blocking or inhibiting the expression of a gene in the target cell. For example, the NOI may be an antisense sequence. The inhibition of gene expression using antisense technology is well known.

The NOI or a sequence derived therefrom may be capable of “knocking out” the expression of a particular gene in the target cell. There are several “knock out” strategies known in the art. For example, the NOI may be capable of integrating in the genome of a neuron so as to disrupt expression of the particular gene. The NOI may disrupt expression by, for example, introducing a premature stop codon, by rendering the downstream coding sequence out of frame, or by affecting the capacity of the encoded protein to fold (thereby affecting its function).

Alternatively, the NOI may be capable of enhancing or inducing ectopic expression of a gene in the target cell. The NOI or a sequence derived therefrom may be capable of “knocking in” the expression of a particular gene.

In one preferred embodiment, the NOI encodes a ribozyme. Ribozymes are RNA molecules that can function to catalyse specific chemical reactions within cells without the obligatory participation of proteins. For example, group I ribozymes take the form of introns which can mediate their own excision from self-splicing precursor RNA. Other ribozymes are derived from self-cleaving RNA structures which are essential for the replication of viral RNA molecules. Like protein enzymes, ribozymes can fold into secondary and tertiary structures that provide specific binding sites for substrates as well as cofactors, such as metal ions. Examples of such structures include hammerhead, hairpin or stem-loop, pseudoknot and hepatitis delta antigenomic ribozymes have been described.

Each individual ribozyme has a motif which recognises and binds to a recognition site in a target RNA. This motif takes the form of one or more “binding arms” but generally two binding arms. The binding arms in hammerhead ribozymes are the flanking sequences Helix I and Helix III which flank Helix II. These can be of variable length, usually between 6 to 10 nucleotides each, but can be shorter or longer. The length of the flanking sequences can affect the rate of cleavage. For example, it has been found that reducing the total number of nucleotides in the flanking sequences from 20 to 12 can increase the turnover rate of the ribozyme cleaving a HIV sequence, by 10-fold (Goodchild, J V K, 1991 Arch Biochem Biophys 284: 386-391). A catalytic motif in the ribozyme Helix II in hammerhead ribozymes cleaves the target RNA at a site which is referred to as the cleavage site. Whether or not a ribozyme will cleave any given RNA is determined by the presence or absence of a recognition site for the ribozyme containing an appropriate cleavage site.

Each type of ribozyme recognizes its own cleavage site. The hammerhead ribozyme cleavage site has the nucleotide base triplet GUX directly upstream where G is guanine, U is uracil and X is any nucleotide base. Hairpin ribozymes have a cleavage site of BCUGNYR, where B is any nucleotide base other than adenine, N is any nucleotide, Y is cytosine or thymine and R is guanine or adenine. Cleavage by hairpin ribozymes takes places between the G and the N in the cleavage site.

More details on ribozymes may be found in “Molecular Biology and Biotechnology” (Ed. R A Meyers 1995 VCH Publishers Inc p 831-8320 and in “Retroviruses” (Ed. J M Coffin et al. 1997 Cold Spring Harbour Laboratory Press pp 683).

Expression of the ribozyme may be induced in all cells, but will only exert an effect in those in which the target gene transcript is present.

Alternatively, instead of preventing the association of the components directly, the substance may suppress the biologically available amount of a polypeptide of the invention. This may be by inhibiting expression of the component, for example at the level of transcription, transcript stability, translation or post-translational stability. An example of such a substance would be antisense RNA or double-stranded interfering RNA sequences which suppresses the amount of mRNA biosynthesis.

In another preferred embodiment, the NOI comprises an siRNA. Post-transcriptional gene silencing (PTGS) mediated by double-stranded RNA (dsRNA) is a conserved cellular defense mechanism for controlling the expression of foreign genes. It is thought that the random integration of elements such as transposons or viruses causes the expression of dsRNA which activates sequence-specific degradation of homologous single-stranded mRNA or viral genomic RNA. The silencing effect is known as RNA interference (RNAi). The mechanism of RNAi involves the processing of long dsRNAs into duplexes of 21-25 nucleotide (nt) RNAs. These products are called small interfering or silencing RNAs (siRNAs) which are the sequence-specific mediators of mRNA degradation. In differentiated mammalian cells dsRNA >30 bp has been found to activate the interferon response leading to shut-down of protein synthesis and non-specific mRNA degradation. However this response can be bypassed by using 21 nt siRNA duplexes allowing gene function to be analysed in cultured mammalian cells.

In one embodiment an RNA polymerase III promoter, e.g., U6, whose activity is regulated by the presence of tetracycline may be used to regulate expression of the siRNA.

In another embodiment the NOI comprises a micro-RNA. Micro-RNAs are a very large group of small RNAs produced naturally in organisms, at least some of which regulate the expression of target genes. Founding members of the micro-RNA family are let-7 and lin-4. The let-7 gene encodes a small, highly conserved RNA species that regulates the expression of endogenous protein-coding genes during worm development. The active RNA species is transcribed initially as an ˜70 nt precursor, which is post-transcriptionally processed into a mature ˜21 nt form. Both let-7 and lin-4 are transcribed as hairpin RNA precursors which are processed to their mature forms by Dicer enzyme.

In a further embodiment the NOI comprises double-stranded interfering RNA in the form of a hairpin. The short hairpin may be expressed from a single promoter, e.g., U6. In an alternative embodiment an effective RNAi may be mediated by incorporating two promoters, e.g., U6 promoters, one expressing a region of sense and the other the reverse complement of the same sequence of the target. In a further embodiment effective or double-stranded interfering RNA may be mediated by using two opposing promoters to transcribe the sense and antisense regions of the target from the forward and complementary strands of the expression cassette.

In another embodiment the NOI may encode a short RNA which may act to redirect splicing (‘exon-skipping’) or polyadenylation or to inhibit translation.

The NOI may also be an antibody. As used herein, “antibody” includes a whole immunoglobulin molecule or a part thereof or a bioisostere or a mimetic thereof or a derivative thereof or a combination thereof. Examples of a part thereof include: Fab, F(ab)′₂, and Fv. Examples of a bioisostere include single chain Fv (ScFv) fragments, chimeric antibodies, bifunctional antibodies.

Transduced target cells which express a particular gene, or which lack the expression of a particular gene have applications in drug discovery and target validation. The expression system could be used to determine which genes have a desirable effect on target cells, such as those genes or proteins which are able to prevent or reverse the triggering of apoptosis in the cells. Equally, if the inhibition or blocking of expression of a particular gene is found to have an undesirable effect on the target cells, this may open up possible therapeutic strategies which ensure that expression of the gene is not lost.

The present invention may therefore be used in conjunction with disease models, such as experimental allergic encephalomyelitis, which is the animal model of Multiple Sclerosis, and experimental autoimmune neuritis which is the animal model of acute and chronic inflammatory demyelinating polyneuropathy. Other disease models are known to those skilled in the art.

An NOI delivered by the vector delivery system may be capable of immortalising the target cell. A number of immortalisation techniques are known in the art (see for example Katakura Y et al. (1998) Methods Cell Biol. 57:69-91).

The vector delivery system can be a non-viral delivery system or a viral delivery system.

In some preferred aspects, the vector delivery system is a viral delivery vector system.

In some further preferred aspects, the vector delivery system is a retroviral vector delivery system.

The term “immortalised” is used herein to cells capable of growing in culture for greater than 10 passages, which may be maintained in continuous culture for greater than about 2 months.

Immortalised motor and sensory neurons and brain cells are useful in experimental procedures, screening programmes and in therapeutic applications. For example, immortalised dopaminergic neurones may be used for transplantation, for example to treat Parkinson's disease.

An NOI delivered by the vector delivery system may be a selection gene, or a marker gene. Many different selectable markers have been used successfully in retroviral vectors. These are reviewed in “Retroviruses” (1997 Cold Spring Harbour Laboratory Press Eds: J M Coffin, S M Hughes, H E Varmus pp 444) and include, but are not limited to, the bacterial neomycin and hygromycin phosphotransferase genes which confer resistance to G418 and hygromycin respectively; a mutant mouse dihydrofolate reductase gene which confers resistance to methotrexate; the bacterial gpt gene which allows cells to grow in medium containing mycophenolic acid, xanthine and aminopterin; the bacterial hisD gene which allows cells to grow in medium without histidine but containing histidinol; the multidrug resistance gene (mdr) which confers resistance to a variety of drugs; and the bacterial genes which confer resistance to puromycin or phleomycin. All of these markers are dominant selectable and allow chemical selection of most cells expressing these genes.

An NOI delivered by the vector delivery system may be a therapeutic gene—in the sense that the gene itself may be capable of eliciting a therapeutic effect or it may code for a product that is capable of eliciting a therapeutic effect.

The term “mimetic” relates to any chemical which may be a peptide, polypeptide, antibody or other organic chemical which has the same binding specificity as the antibody.

The term “derivative” as used herein includes chemical modification of an antibody. Illustrative of such modifications would be replacement of hydrogen by an alkyl, acyl, or amino group.

### Diseases

In general terms the invention is useful for obtaining good distribution of an expressed protein, for example by administering the vector at one site, the protein may be released such that it affects other parts of the brain and nervous system.

The vector system used in the present invention is particularly useful in treating and/or preventing a disease which is associated with the death or impaired function of cells of the nervous tissue, such as neurons, CSF and/or brain cells including glial cells. Thus, the vector system is useful in treating and/or preventing neurodegenerative diseases.

In particular, the vector system used in the present invention may be used to treat and/or prevent a disease which is associated with the death or impaired function of motor or sensory neurons.

Diseases which may be treated include, but are not limited to: pain; movement disorders such as Parkinson's disease, motor neuron diseases including amyotrophic lateral schlerosis (ALS or Lou Gehrig's Disease) and Huntington's disease; Alzheimer's Disease; Spinal Muscle Atrophy and Lysosomal Storage Diseases.

Amyotrophic lateral schlerosis (ALS) is a degenerative disorder of motorneurons with a yearly incidence of 1-2 per 100,000. It is characterised by degeneration of motorneurons in the spinal cord, brain stem and motor cortex which leads to wasting and weakness of limb, bulbar and respiratory muscles. Approximately 5-10% of ALS is familial. Genes whose mutations or haplotypes are thought to play a role in disease predisposition include SOD1, ALS2 and VEGF (Lambrechts et al. Nature Genetics 2003; published on line 6 Jul. 2003 (10.1038/ng1211); Oosthuyse et al. Nature Genetics 2001; June; Vol 28 pages 131-138).

In particular, the vector system used in the present invention is useful in treating and/or preventing ALS. In this embodiment, the NOI may be capable of knockdown of SOD1. Other NOI(s) may encode molecules which prevent apoptosis and therefore prevent cells from dying. Suitable molecules include XIAP and NAIP. Alternatively, NOI(s) may encode neurotrophic molecules which stimulate regeneration such as IGF-1, GDNF, VEGF and cardiotrophin (CT1).

Lysosomal Storage Diseases or Glycolipid Storage Disorders are genetic diseases that result when the rate of glycolipid synthesis is not balanced with the rate of degradation within the cells. As a result, undegraded glycolipids build up in the lysosomes. Such disorders include Fabry Disease, Niemann-Pick diseases, Gangliosidosis, Metachromatic Leukodystrophy and many types of Mucopolysaccharidosis.

Spinal Muscular Atrophy (SMA) is a disease of the anterior horn cells and is an autosomal recessive disease. Anterior horn cells are located in the spinal cord. SMA affects the voluntary muscles for activities such as crawling, walking, head and neck control and swallowing. Categories of SMA include: Type I SMA also called Werdnig-Hoffmann Disease, Type II, Type III, often referred to as Kugelberg-Welander or Juvenile Spinal Muscular Atrophy, Type IV (Adult Onset) and Adult Onset X-Linked SMA. This form also known as Kennedy's Syndrome or Bulbo-Spinal Muscular Atrophy. SMA is a common motor neuron disease in humans and its most severe form causes death by the age of 2 years. It is caused by mutations in the telomeric survival motor neuron gene, SMN1. In particular, the vector system used in the present invention is useful in treating and/or preventing SMA. In this embodiment, the NOI may be capable of encoding a gene for replacement of defective SMN1 gene. Other NOI(s) may encode molecules which prevent apoptosis and therefore prevent cells from dying. Suitable molecules include XIAP and NAIP. Alternatively, NOI(s) may encode neurotrophic molecules which stimulate regeneration such as IGF-1, GDNF, neurotrophin-3 (NT-3), VEGF and cardiotrophin (CT1).

In another embodiment, the vector system used in the present invention is useful in treating and/or preventing Parkinson's disease. In this embodiment, the NOI is capable of encoding a neuroprotective or antiapoptotic molecule. In particular, the NOI(s) may encode molecules which prevent TH-positive neurons from dying or which stimulate regeneration and functional recovery in the damaged nigrostriatal system. The survival of cells during programmed cell death depends critically on their ability to access “trophic” molecular signals derived primarily from interactions with other cells. For example, the NOI can encode a neurotrophic factor, such as ciliary neurotrophic factor (CNTF), glial cell-derived neurotrophic factor (GDNF), or may be a gene involved in control of the cell-death cascade (e.g. Bcl-2). Azzouz et al. (Human Molec. Genet. 9(5):803-811; 2000) have demonstrated increased motoneuron survival and improved neuromuscular function in a mouse model of ALS using a vector containing Bcl-2, suggesting that this technology will be useful in therapeutic strategies involving arresting neuronal and glial cell death induced by injury, disease, and/or aging in humans.

In another preferred embodiment, the NOI is capable of encoding an enzyme or enzymes responsible for L-DOPA or dopamine synthesis such as tyrosine hydroxylase (TH), GTP-cyclohydrolase I, aromatic amino acid dopa decarboxylase, and vesicular monoamine transporter 2 (VMAT2). One aspect of the invention is a viral genome comprising an NOI encoding aromatic amino acid dopa decarboxylase and an NOI encoding VMAT2. Such a genome can be used in the treatment of Parkinson's disease, in particular, in conjunction with peripheral administration of L-DOPA. The sequences of TH, GTP-cyclohydrolase 1 and aromatic amino acid dopa decarboxylase are available under Accession Nos. X05290, U19523 and M76180, respectively.

The vector system of the present invention may also be used in the treatment and/or prevention of an inflammatory neurological disorder including an autoimmune neurological disease.

The inflammatory response evolved to protect organisms against injury and infection. Following an injury or infection a complex cascade of events leads to the delivery of blood-borne leukocytes to sites of injury to kill potential pathogens and promote tissue repair. However, the powerful inflammatory response has the capacity to cause damage to normal tissue, and dysregulation of the innate immune response is involved in different pathologies. It is known that Multiple Sclerosis (MS) is an inflammatory disease of the brain but it has now been suggested that inflammation may significantly contribute to diseases such as stroke, traumatic brain injury, HIV-related dementia, Alzheimer's disease and prion disease.

As mentioned above, MS is a chronic inflammatory disease of the CNS and is presumed to have an autoimmune etiology. MS is believed to be caused by blood-derived T cells specific for CNS antigens. These T cells induce the production in the CNS of antigen-nonspecific mononuclear cells able to destroy oligodendrocytes directly and/or by releasing substances toxic to myelin.

Other autoimmune neurological diseases include the Guillain-Barre syndrome, myasthenia gravis, acute disseminated encephalomyelitis, the stiff-man syndrome, autoimmune neuritis, motor dysfunction, chronic inflammatory demyelinating polyradiculoneuropathy, multifocal motor neuropathy, paraproteinaemic neuropathy, autoimmune diseases of the neuromuscular junction and other disorders of the motor unit, inflammatory myopathy, autoimmune myositis, a parameoplastic neurological disorder, neurological complications of connective tissue diseases and vasculitis.

In one embodiment related to the treatment and/prevention of inflammatory disorders, the nucleotide of interest delivered by the vector system used in the present invention encodes an anti-inflammatory molecule, such as an anti-inflammatory cytokine, or a molecule capable of upregulating the anti-inflammatory molecule. Thus, one embodiment of the present invention relates to a therapeutic approach in neurological inflammatory disorders, such as MS, which involves the delivery of an anti-inflammatory molecule directly to the CNS.

Cytokines which may be useful in the treatment of MS and possible other disorders include IL-1β, IL-2, IL-4, IL-6, IL-1n, IFN-β, IFN-γ, TNF-α, p55TNFR-Ig, p75dTNFR, TGF-β, PDGF-α and NGF. More generally, it will be appreciated that anti-inflammatory cytokines may be useful delivered in accordance with the present invention in the treatment and/or prevention of neurological inflammatory diseases.

Another approach involves the delivery of a nucleotide of interest which inhibits, or encodes a molecule which inhibits, a pro-inflammatory molecule, such as an inflammatory cytokine. Thus the use of inhibitors, such as those described above, e.g. ribozymes, siRNA, antibodies and antisense sequences, is envisaged.

A further approach involves the delivery of myelin proteins and or growth factors for rebuilding and or regenerating the damaged neuron myelin sheath.

In addition, the capacity to target sensory neurons makes the system attractive for use in pain relief. There are also potential applications in hyperanalgesia. For example, encephalins may be used to re-grow sensory neurons in conditions such as paraplegia. The vector system could be used to provide RARβ2 at the target site. As such, one embodiment of the present invention provides a method for treating and/or preventing pain using RARβ2 and/or an agonist thereof such as retinoic acid and/or CD2019. In a preferred embodiment, pain may be a symptom of or associated with e.g., a neurological disorder or neurological injury. In a preferred embodiment, RARβ2 is delivered using a lentiviral vector, and more preferably, the lentiviral vector is pseudotyped with rabies G, or a mutant, variant, fragment, or homologue thereof. Teachings relating to the use of RARβ2 and agonists thereof for neurite outgrowth and/or neurite regeneration can be found in WO00/175135 and in WO00/057900.

Table 3 summarises a number of examples of diseases that may be treated using the methods and vectors of the present invention along with suggested mechanisms for treatment plus examples of the types of genes that could be modulated in order to treat the disease.

TABLE 3

| | Mechanism Of | | Preferred Site |
|---|---|---|---|
| Disease | Treatment | Gene(s) | Of Therapy |
| Pain (cancer) | Interrupt signalling | Enkephalin, beta endorphin | Intraspinal, |
| | | GDNF, ion channel | Intrathecal, |
| | | hyperpolarization | Amygdala |
| Pain (diabetic) | As above or promote | As above or RARβ2 | DRG, skin |
| | neurite outgrowth or | | |
| | regeneration | | |
| Pain (herpetic neuralgia) | As above | As above | Lesions |
| Alzheimer's | | NGF | Cortex |
| Parkinson's | Dopamine replacement | ADCC, TH, CH1, VMAT2, | Striatum |
| | | etc. | |
| Parkinson's | Decrease rate of death of | GDNF, nurturin, other | Striatum, |
| | dopaminergic neurones | | Nigra |
| Childhood | Avoid diabetic sequellae | Vasopressin | Hypothalamus, |
| craniopharyngeoma | | | Pituitary? |
| Glioma | Destroy residual tumor | Prodrug activating enzyme | Glioma bed |
| | after excision | (TK, Cyt P450), | |
| | | Angiostatics | |
| Diabetic Retinopathy | Arrest blood vessel | Angiostatics, e.g Endostatin | Retina |
| | proliferation | and/or Angiostatin, | |
| | | PEDF | |
| | | Flt-1 | |
| Macular degeneration | Arrest degeneration | Growth factors | Retina |
| Retinitis pigmentosa | Arrest degeneration | XIAP, | Retina, vitreus |
| | | Growth factors | |
| Huntington's Disease | Avoid PolyG intracellular | CNTF, scAb against | Striatum |
| | effects | polyGlut, CREB factor | |
| Spinal muscular atrophy | Replace missing protein | SMN1, SMN 2 | Intraspinal |
| | | growth factor: GDNF, IGF- | Muscle (retrograde) |
| | | I, VEGF, NT-3, CT-I | |
| ALS | Arrest degeneration | SOD1 knockdown | Intraspinal |
| | | (genetic form) by | Muscle (retrograde) |
| | | RNAi/antisense, growth | |
| | | factor: GDNF, IGF-I, | |
| | | VEGF, NT-3, CT-I, bcl-2 | |
| Spinal cord regeneration | Promote regrowth, | NT3, antiNogo Antibodies, | Spinal cord, |
| | remove inhibitors of | Growth factors: GDNF, | Intrathecal |
| | regrowth | IGF-I. RARβ2 | |
| Multiple sclerosis | Prevent demyelination | Cytokines | Intrathecal |
| Lysosomal storage with | Replacement with protein | Beta glucuronidase, | Intracerebral, |
| neurological involvement | capable of cellular uptake | Other | Intraventricular |
| Stroke | Protect neural tissue in | EPO/other using HREs | Intrathecal |
| | anticipation of second | | |
| | episode | | |

In addition, the observation that retrograde transport to the brain occurs following subretinal delivery can be exploited to deliver a gene to treat any disorder affecting regions of the optic nerve, optic chiasm, optic tract or region of LGN (Lateral Geniculate Nucleus). Such disorders include (but are not limited to) glaucoma or other disorders that are secondary to an elevation in intraocular pressure, neuronal dystrophies such as multiple sclerosis. Suitable genes for expression include growth or survival factors such as erythropoietin or VEGF for the treatment of stroke, expression of neuroprotective factor such as PEDF, GDNF or neurotrophins for the treatment of optic neuropathies (e.g. Leber's congenital disease).

In particular, in a preferred embodiment for treating motor neuron diseases, the vector system is a lentiviral vector system because advantageously with the use of a lentiviral vector system having a rabies G pseudotype, one achieves high efficiency retrograde transport and long term expression. While both adenovirus and HSV and even AAV (to a lesser extent) do get retrogradely transported, the lentiviral vector system having a rabies G pseudotype achieves high efficiency retrograde transport through the selective transduction of neurons. Advantageously, lentiviral vectors pseudotyped with rabies G specifically target motor neurons with high efficiency. Moreover, the use of lentiviral vectors avoids the toxicity issues common to the use of adenovirus and HSV, for example. It is a further advantage of a lentiviral vector system pseudotyped with a rabies glycoprotein G that retrograde transport occurs through the intramuscular route with little to no transduction of adult muscle cells (Mazarakis et al., Supra) thereby exhibiting the selectivity necessary for efficient transduction of motor neurons, whereas the use of AAV may not be so selective in that transduction of motor neurons also results in long-lasting expression in the muscle (Lu et al. Neurosci. Res. 2003 January; 45(1): 33-40).

### Pharmaceutical Compositions

The present invention also provides the use of a vector delivery system in the manufacture of a pharmaceutical composition. The pharmaceutical composition may be used to deliver an EOI, such as an NOI, to a target cell in need of same.

The vector delivery system can be a non-viral delivery system or a viral delivery system. In some preferred aspects, the vector delivery system is a viral delivery vector system. In some further preferred aspects, the vector delivery system is a retroviral vector delivery system, preferably, a lentiviral vector delivery system.

The pharmaceutical composition may be used for treating an individual by gene therapy, wherein the composition comprises or is capable of producing a therapeutically effective amount of a vector system according to the present invention.

The method and pharmaceutical composition of the invention may be used to treat a human or animal subject. Preferably the subject is a mammalian subject. More preferably the subject is a human. Typically, a physician will determine the actual dosage which will be most suitable for an individual subject and it will vary with the age, weight and response of the particular patient.

The composition may optionally comprise a pharmaceutically acceptable carrier, diluent, excipient or adjuvant. The choice of pharmaceutical carrier, excipient or diluent can be selected with regard to the intended route of administration and standard pharmaceutical practice. The pharmaceutical compositions may comprise as (or in addition to) the carrier, excipient or diluent, any suitable binder(s), lubricant(s), suspending agent(s), coating agent(s), solubilising agent(s), and other carrier agents that may aid or increase the viral entry into the target site (such as for example a lipid delivery system).

Where appropriate, the pharmaceutical compositions can be administered by any one or more of: inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intracavernosally, intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

The vector system used in the present invention may conveniently be administered by direct injection into the patient. For the treatment of neurodegenerative disorders, such as Parkinson's disease, the system may be injected into the brain. The system may be injected directly into any target area of the brain (for example, the striatum or substantia nigra). Alternatively, the system can be injected into a given area, and the target area transduced by retrograde transport of the vector system. Intramuscular injection is particularly preferred as the least invasive method of treatment.

Table 3 outlines preferred sites for administering therapy by injection and includes intraspinal, intrathecal, amygdala, DRG, skin, sites of lesions of herpetic neuralgia, cortex, striatum, nigra, hypothalamus, pituitary, glioma bed, retina, vitreus, muscle, spinal cord and intraventricular injection.

### Transport

The present invention provides the use of a vector system to transduce a target site, wherein the vector system travels to the site by retrograde transport.

A virus particle may travel in the same direction as a nerve impulse, i.e. from the cell body, along the axon to the axon terminals. This is known as anterograde transport.

The present inventors have shown that vector systems comprising protein of the present invention are transported in a retrograde manner, in the opposite direction of anterograde transport. Retrograde transport (or transfer) of a vector means that it is taken up by the axon terminals and travels toward the cell body. The precise mechanism of retrograde transport is unknown, however. It is thought to involve transport of the whole viral particle, possibly in association with an internalised receptor.

The movement of membranous organelles at 50-200 mm per day toward the synapse (anterograde) or back to the cell body (retrograde) occurs via “fast transport” (Hirokawa (1997) Curr Opin Neurobiol 7(5):605-614). The fact that the present vector systems can be specifically transported in this manner (as demonstrated herein) suggests that the env protein may be involved.

HSV, adenovirus and hybrid HSV/adeno-associated virus vectors have all been shown to be transported in a retrograde manner in the brain (Horellou and Mallet (1997) Mol Neurobiol 15(2) 241-256; Ridoux et al. (1994) Brain Res 648:171-175; Constantini et al. (1999) Human Gene Therapy 10:2481-2494). Injection of Adenoviral vector system expressing glial cell line derived neurotrophic factor (GDNF) into rat striatum allows expression in both dopaminergic axon terminals and cell bodies via retrograde transport (Horellou and Mallet (1997) as above; Bilang-Bleuel et al. (1997) Proc. Natl. Acd. Sci. USA 94:8818-8823).

Retrograde transport can be detected by a number of mechanisms known in the art. In the present examples, a vector system expressing a heterologous gene is injected into the striatum, and expression of the gene is detected in the substantia nigra. It is clear that retrograde transport along the neurons which extend from the substantia nigra to the basal ganglia is responsible for this phenomenon. It is also known to monitor labelled proteins or viruses and directly monitor their retrograde movement using real time confocal microscopy (Hirokawa (1997) as above).

By retrograde transport, it is possible to get expression in both the axon terminals and the cell bodies of transduced neurons. These two parts of the cell may be located in distinct areas of the nervous system. Thus, a single administration (for example, injection) of the vector system of the present invention may transduce many distal sites.

The present invention also provides the use of a vector system of the present invention to transduce a target site, which comprises the step of administration of the vector system to an administration site which is distant from the target site to achieve good penetration and biodistribution throughout the CNS. For example, administration to the one area of the brain may give rise to distribution of the EOI is different parts of the brain and/different cell types.

The target site may be any site of interest. It may or may not be anatomically connected to the administration site. The target site may be capable of receiving vector from the administration site by axonal transport, for example anterograde or (more preferably) retrograde transport. For a given administration site, a number of potential target sites may exist which can be identified using tracers by methods known in the art (Ridoux et al. (1994) as above).

For example, intrastriatal injection of HSV/AAV amplicon vectors causes transgene expression in the substantia nigra, cortex, several thalamic nuclei (posterior, paraventricular, parafasicular, reticular), prerubral filed, deep mesencephalic nuclei, mesencephalic grey nucleus, and intrastitial nucleus of the medial as well as dorsal longitudinal fasiculus (Constanti et al. (1999) as above). In addition, intrastriatal injection of CVS/EIAV vectors causes transgene expression in the globus pallidus, cortex, various thalamic nuclei, amygdala, hypothalamus, supraoptic nucleus, deep mesencepthalic nuclei, substantia nigra, caudal regions of the brainstem such as the nuclei of the brachium inferior colliculus, paraleminiscal nuclei, genic nuclei, parabrachial nuclei, ventral cochlear nuclei and facial nuclei.

A target site is considered to be “distant from the administration” if it is (or is mainly) located in a different region from the administration site. The two sites may be distinguished by their spatial location, morphology and/or function.

In the brain, the basal ganglia consist of several pairs of nuclei, the two members of each pair being located in opposite cerebral hemispheres. The largest nucleus is the corpus striatum which consists of the caudate nucleus and the lentiform nucleus. Each lentiform nucleus is, in turn, subdivided into a lateral part called the putamen and a medial part called the globus pallidus. The substantia nigra and red nuclei of the midbrain and the subthalamic nuclei of the diencephalon are functionally linked to the basal ganglia. Axons from the substantia nigra terminate in the caudate nucleus or the putamen. The subthalamic nuclei connect with the globus pallidus. For conductivity in basal ganglia of the rat see Oorschot (1996) J. Comp. Neurol. 366:580-599.

In a preferred embodiment, the administration site is the striatum of the brain, in particular the caudate putamen. Injection into the putamen can label target sites located in various distant regions of the brain, for example, the globus pallidus, amygdala, subthalamic nucleus or the substantia nigra. Transduction of cells in the pallidus commonly causes retrograde labelling of cells in the thalamus. In a preferred embodiment the (or one of the) target site(s) is the substantia nigra.

In another embodiment, the vector system is injected directly into the spinal cord. This administration site accesses distal connections in the brain stem and cortex.

Within a given target site, the vector system may transduce a target cell. The target cell may be a cell found in nervous tissue, such as a sensory or motor neuron, astrocyte, oligodendrocyte, microglia or ependymal cell. In a preferred embodiment, the target site is a neuron, for example, a TH positive neuron.

The vector system is preferably administered by direct injection. Methods for injection into the brain (in particular the striatum) are well known in the art (Bilang-Bleuel et al. (1997) Proc. Acad. Natl. Sci. USA 94:8818-8823; Choi-Lundberg et al. (1998) Exp. Neurol. 154:261-275; Choi-Lundberg et al. (1997) Science 275:838-841; and Mandel et al. (1997)) Proc. Acad. Natl. Sci. USA 94:14083-14088). Stereotaxic injections may be given.

As mentioned above, for transduction in tissues such as the brain, it is necessary to use very small volumes, so the viral preparation is concentrated by ultracentrifugation. The resulting preparation should have at least 10⁸ t.u./ml, preferably from 10⁸ to 10¹⁰ t.u./ml, more preferably at least 10⁹ t.u./ml. (The titer is expressed in transducing units per ml (t.u./ml) as titred on a standard D17 cell line). It has been found that improved dispersion of transgene expression can be obtained by increasing the number of injection sites and decreasing the rate of injection (Horellou and Mallet (1997) as above). Usually between 1 and 10 injection sites are used, more commonly between 2 and 6. For a dose comprising 1-5×109 t.u./ml, the rate of injection is commonly between 0.1 and 10 μl/min, usually about 1 μl/min.

We have also shown that following administration to the CSF, e.g. using intrathecal delivery, expression of an NOI may be found in various areas of the brain, such as the ependymal and leptomeningeal cells, hippocampus, corpus collasum and septum, and the spinal cord.

### Transplantation

The present invention also provides an immortalised cell of the CNS such as a sensory or motor neuron or brain cell and its use in transplantation methods.

Grafting protocols using embryonic dopaminergic neurons, equivalent cells from other species, and neural progenitor cells are known (reviewed in Dunnett and Bjorklund (1999) Nature Vol 399 Supplement pages A32-39). Similar techniques could be used for grafting the cells of the present invention.

The present invention will now be further described by way of the following non-limiting examples, provided for illustrative purposes only.

### EXAMPLES

In addition to the disclosure provided below, details of the EIAV vector system used in the Examples, its production and viral transduction methods can be found in Mazarakis et al. (2001) ibid and WO02/36170 which are herein incorporated by reference, and in particular, the Materials and Methods section of Mazarakis et al. (2001) and the Examples section of WO02/36170.

### Example 1

### Transduction of Presumptive Dopaminergic (TH+) Neurons in Rodent Mesencephalic Cultures

Methods

Mesencephalic cultures: Cultures are prepared exactly as described by Lotharius et al. (1999) (J. NeuroSci. 19:1284-1293). Briefly, the ventral mesencephalon was removed from embryonic day 14 (E14) CF1 murine embryos (Charles River Laboratories, Willington, Mass.). Tissues are mechanically dissociated, incubated with 0.25% trypsin and 0.05% DNase in phosphate buffered saline (PBS) for 30 minutes at 37° C., and further triturated using a constricted Pasteur pipette. For immunocytochemistry, cells are plated at a density of 50,000 cells per 35 mm microwell plate (1.25×10³ cells/mm²). All plates are pre-coated overnight with 0.5 mg/ml poly-d-lysine followed by 2.5 mg/ml laminin for 2 hours at room temperature. Initial plating is done in serum-containing medium consisting of 10% fetal calf serum in DMEM:F1 supplemented with B27 additive (Life Technologies, Gaithersburg, Md.), 6 g/L glucose, and antibacterial agents. Glial numbers are reduced by subsequently maintaining cells in serum-free Neurobasal medium (Life Technologies) supplemented with 0.5 mM L-glutamine, 0.01 mg/ml streptomycin/100 units penicillin, and 1× B27 supplement. Half of the culture medium is replaced with fresh Neurobasal medium every 48 hours.

DA Release: In order to measure dopamine uptake, release and content cells are plated at a density of 400,000 cells per 16 mm well (2×10³ cells/mm²). To measure DA release, cells are loaded with 2.4*Ci/ml ³H-DA/KRS for 20 min. at 37° C. and washed 3× for 3 min. Radioactive counts from a wash sample are measured using a Beckman scintillation counter and used as a control for basal levels of 3H-DA release. Cells are then treated with 30 mM K⁺ in KRS (adjusted as described in Dalman & O'Malley, 1999 J. Neurosci 19:5750-5757) for 5 min. and the amount of 3H-DA released during this time period is collected. Subsequently, cultures are washed extensively and lysed in 0.1 N PCA by freeze-thawing, and residual, intracellular 3H-DA is measured. Total ³H-DA uptake is calculated by summation of tritium content from all of the fractions collected, including the acid lysate.

Plasmid Construction

### a) Vector Plasmids

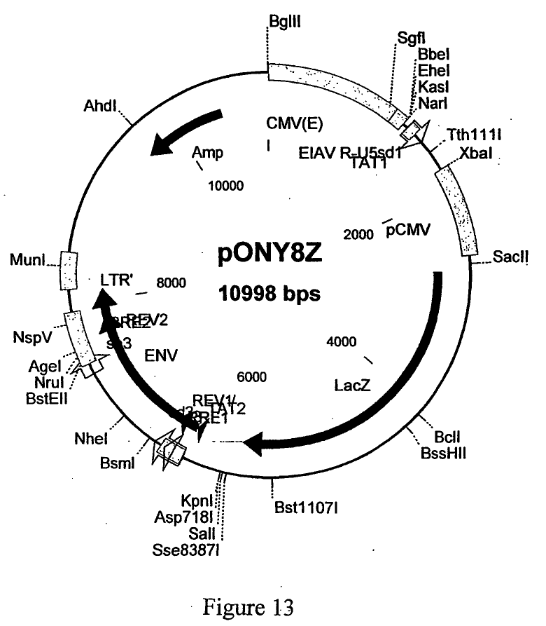

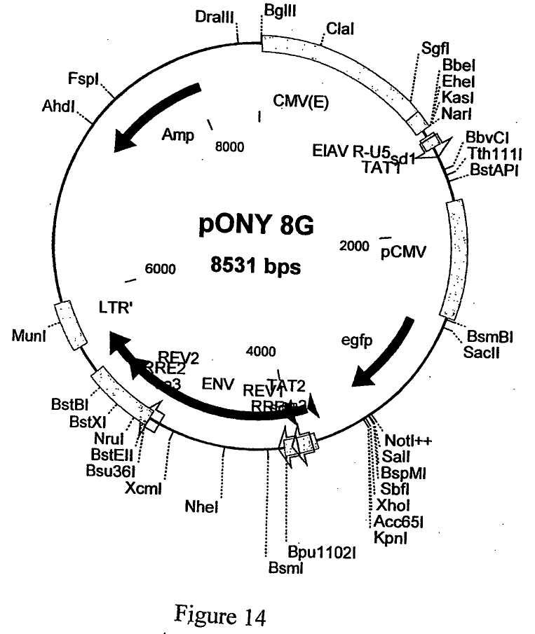

Numbering used is as of Payne et al 1994 (J. Gen Virol. 75:425-429). The pONY series of vectors and their pseudotyping with the different envelopes have been described previously (WO99/61639) (Mitrophanous et al. 1999 Gene Ther 1999 6:1808-1818). pONY8Z (FIG. 13, SEQ ID NO:1) was derived from pONY4.0Z (WO99/32646) by introducing mutations which prevented expression of TAT by an 83 nt deletion in the exon 2 of tat, prevented S2 expression by a 51nt deletion, prevented REV expression by deletion of a single base within exon 1 of rev and prevented expression of the N-terminal portion of gag by insertion of T in the first two ATG codons of gag, thereby changing the sequence to ATTG from ATG. With respect to the wild type EIAV sequence (Acc. No. U01866) these correspond to deletion of nt 5234-5316 inclusive, nt 5346-5396 inclusive and nt 5538. The insertion of T residues was after nt 526 and 543. pONY8.0G (FIG. 14, SEQ ID NO:2) was derived from pONY8Z by exchange of the Lac Z reporter gene for the enhanced green fluorescent protein (GFP) gene. This was done by transferring the Sac II-Kpn I fragment corresponding to the GFP gene and flanking sequences from pONY4.0G (WO99/32646) into pONY8Z cut with the same enzymes.

### b) Envelope Plasmids

pSA91ERAwt was used for pseudotyping with rabies G. This plasmid has been described previously (WO99/61639) under the name “pSA91RbG”. Briefly, pSA91ERAwt was constructed by cloning 1.7 kbp BglII rabies G fragment (strain ERA) from pSG5rabgp (Burger et al., 1991 J. Gen. Virol. 72. 359-367) into pSA91, a derivative of pGW1HG (Soneoka et al 1995 Nucl. Acids Res. 23: 628-633) from which the gpt gene has been removed by digestion with BamHI and re-ligation. This construct, pSA91ERAwt, allows expression of rabies G from the human cytomegalovirus (HCMV) immediate early gene promoter-enhancer.

pRV67 was used for pseudotyping with rabies G. pRV67 (described in WO99/61639) is a VSV-G expression plasmid in which VSV-G was expressed under the control of human cytomegalovirus promoter/enhancer, in place of rabies G in pSA91ERAwt.

Production and Assay of Vectors: Vector stocks were generated by calcium-phosphate transfection of human kidney 293T cells plated on 10 cm dishes with 16 μg of vector plasmid, 16 μg of gag/pol plasmid and 8 μg of envelope plasmid. 36-48 h after transfection, supernatants were filtered (0.45 μm) aliquoted and stored at −70° C. Concentrated vector preparations were made by initial low speed centrifugation 6 000×g (JLA-10.500 for 16 hours at 4° C. followed by ultracentrifugation at 20,000 rpm (SW40Ti rotor) for 90 min., at 4° C. The virus was resuspended in PBS for 3-4 h aliquoted and stored at −70° C. Transduction was carried out in the presence of polybrene (8 μg/ml).

Viral transductions: Transductions were carried out after 7 days in vitro (DIV7). Specifically, culture media were removed and reserved with a small aliquot being added back to cultures following the addition of the indicated viral MOI. Dishes were maintained at 37° C. for 5 hours after which the virus was removed and the wells were washed twice with the reserved conditioned media. Fresh Neuralbasal media was added in a 50:50 ratio and cells were maintained for a further 3 days.

Immunocytochemistry: To determine the effect of viral transductions on dopaminergic cultures plates were processed for TH and GFP immunoreactivity. Briefly, cells were rinsed with PBS, fixed in 4% paraformaldehyde, permeabilized in 1% bovine serum albumin/0.1% Triton-X-100/PBS for 30 minutes at room temperature (RT), and incubated with a mouse monoclonal anti-TH antibody (1:1000; Diastor) as well as a rabbit polyclonal anti-GFP antibody (1:1000; Chemicon) for 1 hr at 37° C. Cells were subsequently incubated with a CY3-conjugated anti-mouse IgG (1:250; Jackson Immunoresearch) and an Alexa-488-conjugated anti-rabbit secondary (1:250; Molecular Probes). Neurons were imaged with a Fluoview confocal microscope (Olympus America Inc). Manual cell counts were conducted as described (Lotharius et al, 1999). Briefly, 6 consecutive fields were assayed per dish leading to the quantification of 200-300 TH neurons per experiment. Experiments were repeated 3 times using cultures isolated from independent dissections. Descriptive statistics (mean +/−SEM) of cell counts were calculated with statistical software (GraphPad Prism Software Inc.)

Results

Comparison of Transduction with EIAV Vectors Pseudotyped with VSVG and Rabies G

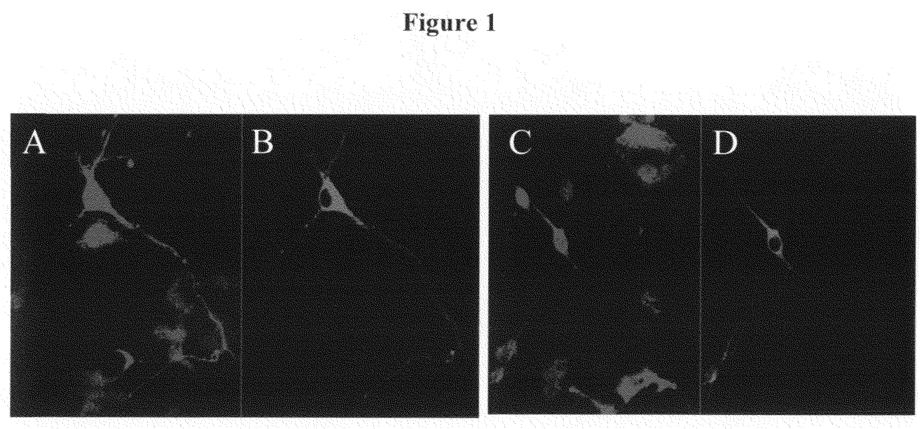

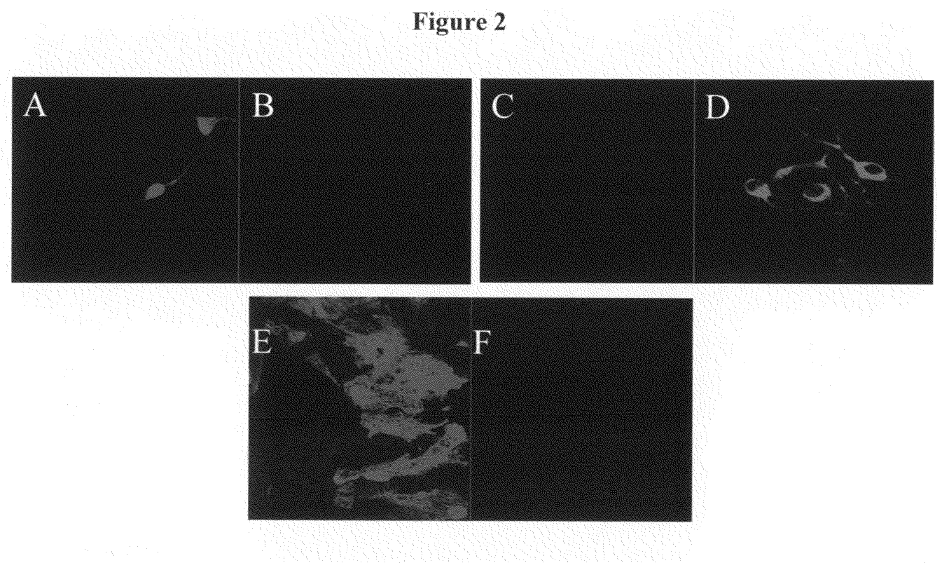

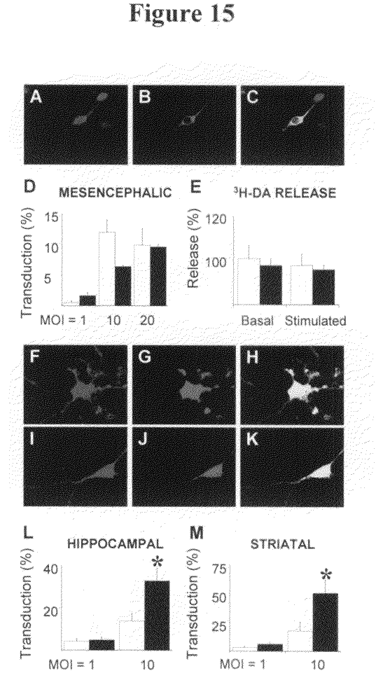

In order to determine whether the equine lentiviral preparations could transduce TH+ neurons in vitro, mesencephalic cultures were prepared and transduced on DIV7. This time point was chosen because it had been previously determined that most characteristic dopaminergic functions were established by then (Lotharius et al., 1999 as above; Dalman and O'Malley, 1999 as above; Lotharius and O'Malley, 2000 J. Biol. Chem. e-publication (ahead of print) 31 Aug. 2000). Both pSA91ERAwt and pRV67 pseudotyped EIAV vectors were capable of transducing dopaminergic neurons in vitro at about 10% efficiency at the highest MOI tried (Table 4, FIG. 1 and FIG. 15A-15D). Both vectors also transduced non-dopaminergic neurons and glial populations as judged by morphological criteria (FIG. 2). In particular the pRV67 vector transduced approximately 80% of the estimated glia/per dish whereas the pSA91ERAwt vector transduced only 5-10%.

TABLE 4
Transduction efficiency of dopaminergic neurons in vitro

| | pSA91ERAwt | pRV67 |
|---|---|---|
| MOI 1 | 1.7 +/− 0.50* | 0.5 +/− 0.30 |
| MOI 10 | 6.5 +/− 0.16 | 12.1 +/− 2.0 |
| MOI 20 | 9.7 +/− 0.42 | 10.0 +/− 2.7 |

*SEM

Functional Analysis of Transduced Cultures Using Uptake and Release of Dopamine Assay

To determine whether viral transduction altered dopaminergic properties the 3H-dopamine (³H-DA) release assay was used. Because dopamine transporters are localized exclusively on dopaminergic neurons in the midbrain (Kuhar et al., 1998 Adn. Pharmacol. 42:1042-5), this approach allows for the selective analysis of dopaminergic function in the midst of a heterogeneous culture system. The data indicate that neither pSA91ERAwt nor pRV67 pseudotyped vectors affected 3H-DA release (Table 5 and FIG. 15E) and this is indicative of not causing an aberration in the function of the TH+ neurons after EIAV vector transduction.

TABLE 5
Effects of viral transduction on DA uptake and release

| | pSA91ERAwt | pRV67 |
|---|---|---|
| Stage | % control | % control |
| Basal Release | 98 +/− 3 | 101 +/− 6 |
| K+-stimulated | 96 +/− 2 | 98 +/− 5 |

Cultures were kept naive or were transduced with the indicated viral particles at an MOI of 20 as described in the Methods. Following transduction the media was removed, and the cultures were washed with KRS and then loaded with 3H-DA. Basal or spontaneous release was measured at 10 min. after exposure to 3H-DA. Release was expressed as a percentage of total uptake SEM. Typically, basal release was 2-3% of the total and K⁺-stimulated release was 5-6% of the total uptake.

Primary cultures of both hippocampal and striatal neurons could also be transduced in vitro by EIAV vectors pseudotyped with either VSV-G or rabies-G. This was demonstrated in hippocampal and striatal neurons by the colocalization of antibody staining for both the reporter protein β-gal and NeuN, a neuronal-specific marker (FIGS. 15F-H and 15I-15K, respectively). At MOIs of 1 and 10, there was no significant difference in transduction efficiency between the hippocampal and striatal neurons (MOI=1, P=0.23 and MOI=10, P=0.81, ANOVA, FIGS. 15L and 15M), although an increase was observed compared to mesencephalic dopaminergic neurons. Similarly, there was no significant difference in transduction efficiency at MOI=1 when vectors are pseudotyped with either VSV-G or rabies-G (P=0.14, ANOVA). However, at an MOI of 10, the transduction efficiency of the rabies-G pseudotyped vector was significantly higher than that observed with the VSV-G pseudotyped vector (P<0.001, ANOVA).

### Example 2

### Transduction of the Adult Rat CNS

Methods

Stereotactic injection into rat brain: In order to examine virally encoded gene expression, EIAVlacZ (pONY8Z) pseudotyped with either VSV-G (pRSV67) or Rabies G (pSA91ERAwt) were stereotaxically microinjected into the adult rat striatum as follows: rats were anesthesized with hypnorm and hypnovel (Wood et al., (1994) Gene Therapy 1:283-291) and injected with 2×1 μl of viral stocks (for EIAV lacZ is typically 1-5×10⁹ t.u./ml for VSV-G and 6×10⁸ t.u/ml for Rabies-G pseudotyped vector) into the striatum, at coordinates: Bregma 3.5 mm lateral, 4.75 mm vertical from dura, and 1 mm rostral, 3.5 mm lateral 4.75 mm vertical using a fine drawn glass micropipette over a period of 2 min. For perinigral (medial lemniscus) injections 2×1 μl of viral stocks were delivered at coordinates: 4.7 mm caudal to Bregma, 2.2 mm lateral, 7 mm vertical from dura and 5.4 caudal, 2.2 lateral and 7.5 mm vertical. The pipette was pulled up 1 mm and left for another 2 min. before retracting slowly to the surface. Animals were analysed 1 and 2 weeks following injection. Rats were perfused with 4% paraformaldehyde (PFA) containing 2 mM MgCl₂ and 5 mM ethylene glycol bis(beta-aminoethylether)-N,N,N′,N′-tetraacetic acid. At different time intervals after the intracranial injections, rats were sacrificed and brains were removed and placed in fixative overnight, submersed in 30% sucrose at 4° C. overnight and frozen on Tissue-Tech OCT embedding compound (Miles Ind. USA). Fifty-micrometer sections were cut on a freezing microtome and floated briefly in PBS-2 mM MgCl₂ at 4° C. as a wash. Expression of lacZ was determined by placing the sections in X-gal staining solution for 3-5 hours.

Immunohistochemistry: To determine whether the cells transduced were neurons or glial-cells a LacZ antibody was used in conjuction with antibodies that recognise either neuronal (NeuN) or glial (GFAP) markers. Double immunostaining was carried out on brain sections. Sections were incubated with rabbit polyclonal LacZ antibody ( 1/100^{th}; 5 prime→3 prime) and mouse monoclonal neurofilament (NeuN) antibody ( 1/50^{th}; Chemicon), or mouse monoclonal GFAP ( 1/50^{th}; Chemicon) at 4° C. overnight in PBS-10% goat serum and 0.5% TritonX-100. Sections were washed with PBS and then incubated with Alexa 488 conjugated goat anti rabbit IgG ( 1/200^{th}; Molecular Probes) or Texas Red-X conjugated goat anti-mouse IgG ( 1/200^{th}; Molecular Probes) at room temperature for 2-3 hours. After washing, the sections were examined under a fluorescence microscope.

Polymerase chain reaction: To detect viral DNA after injection of pONY8Z virus pseudotyped with VSV-G or rabies-G into rat striatum (n=4) (as described above), animals were sacrificed 2 weeks post-transduction. Punches from striatum, thalamus and substantia nigra were quickly removed and frozen in liquid nitrogen. Genomic DNA was isolated from all samples using the Wizard Genomic DNA Purification kit (Promega, Madison-Wisconsin #A1120). Thawed brain tissue (20 mg) was homogenized for 10 seconds using a disposable homogenizer in cooled nuclei lysis solution according to the manufacturer's protocol. PCR reactions were set to detect the E. coli LacZ gene (Gene Bank #V00296) expressed by injected vectors. Each reaction was set in 50 μl volume containing the following components (final concentration): 300 nM forward primer CGT TGC TGC ATA AAC CGA CTA CAC (SEQ ID NO:10; nt: 638-661), 300 nM reverse primer TGC AGA GGA TGA TGC TCG TGA C (SEQ ID NO: 11; nt: 1088-1067) 200 μM of dNTP (each), 2 mM MgCl₂, 1× FastStart Taq DNA polymerase buffer and 2 Units FastStart Taq DNA polymerase (Roche Diagnostics, Mannheim Germany). 300 ng of template DNA was used per reaction. PCR amplification was carried out on a PCR Express (Hybaid, Hercules, USA) under the following thermal cycling conditions: initial denaturation and enzyme activation at 95° C. for 4 minutes, followed by 30 cycles of denaturation at 95° C. for 30 seconds, annealing at 58° C. for 45 seconds and elongation at 72° C. for 45 seconds, and finally, one cycle of extension at 72° C. for 7 minutes. PCR products (10 μl/reaction) were resolved on 1.2% TBE agarose gel at 10 v/cm for 2 hours.

Results

Comparison of Transduction Using EIAV Vectors Pseudotyped with VSVG and Rabies G After Delivery to Striatum

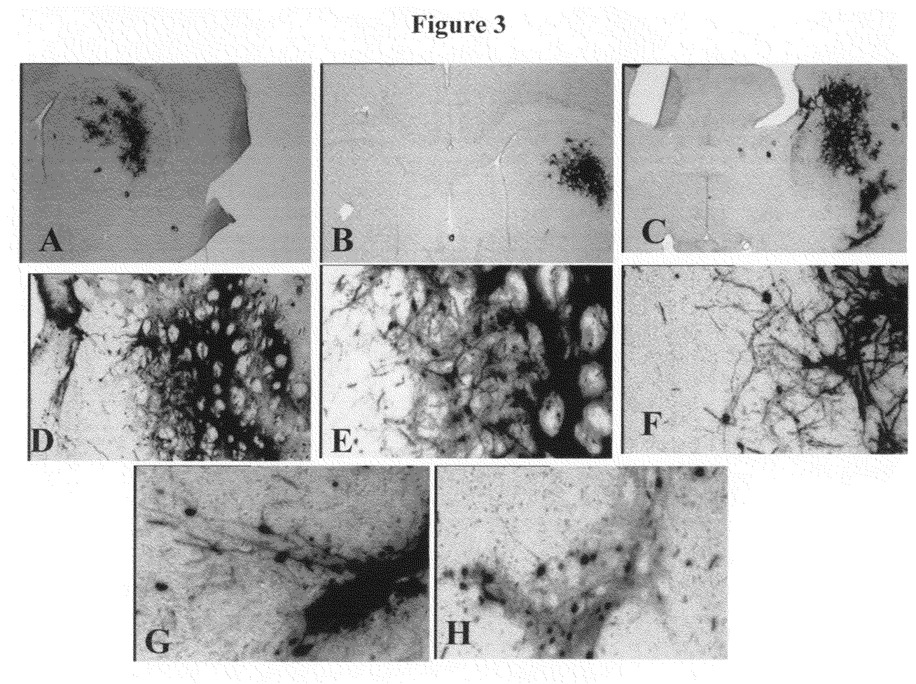

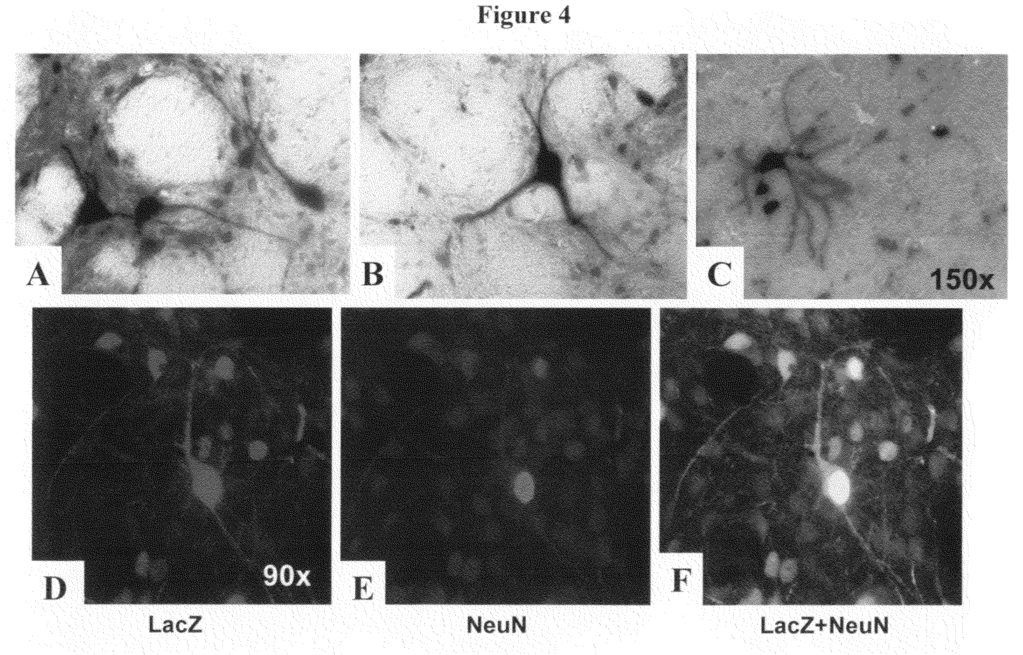

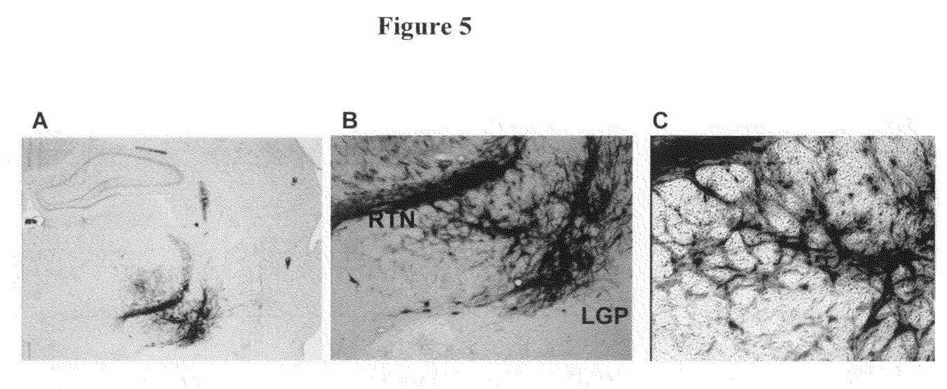

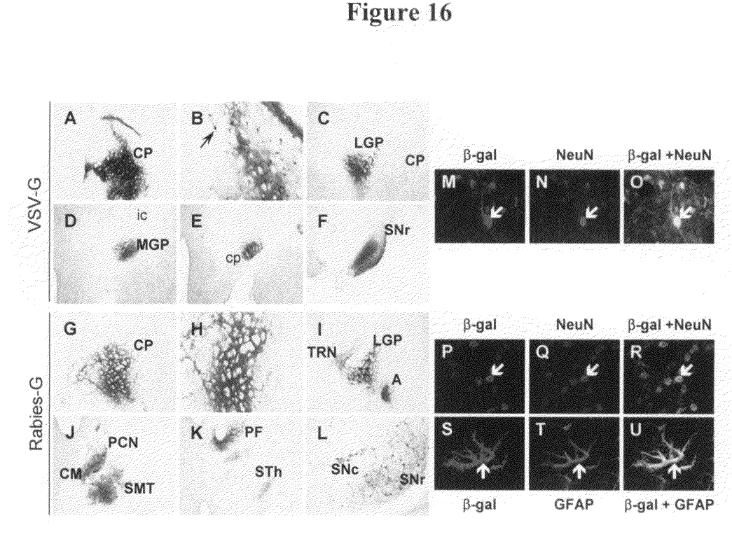

In order to compare the pattern of expression of the two different pseudotyped vectors in the adult rat brain, concentrated viral vector preparations were sterotactically injected into caudate putamen. VSVG pseudotyped EIAV-LacZ expressing vectors gave very efficient gene transfer spanning an average region of 2.5 mm anteroposterior (50×50 μm coronal sections stained), 1 mm mediolateral and 5 mm dorsoventral around the area of injection, giving an approximate cell volume transduced of ˜5×10⁴ (FIG. 3). This equates to about 29750±1488 transduced cells (FIGS. 16A and 16B). The transduced cells have principally neuronal morphology (striatal interneurons, medial spiny neurons and aspiny neurons) which was further confirmed using confocal co-localisation of the neuronal marker NeuN and LacZ markers (FIG. 4 and FIGS. 16M-16O). Transduced glia were seen in some rats in white matter tracts, such as corpus callosum. Transduction was localised to striatum, with some anterograde transport of LacZ proteins to axons projecting to subthalamic nucleus (SN), the lateral and medial globus pallidus (FIGS. 16C and 16D), cerebral penduncle (FIG. 16E), and the substantia nigra pars reticulata (SNr) (FIG. 16F). In rats where lateral globus pallidus (GP) is co-transduced, reticular thalamic nucleus (RTN) was also strongly stained by anterograde transport of LacZ (FIG. 5).

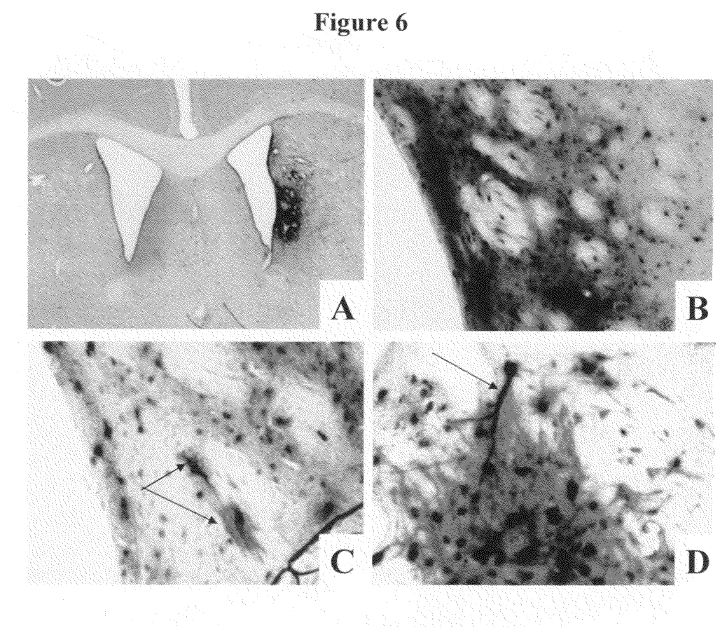

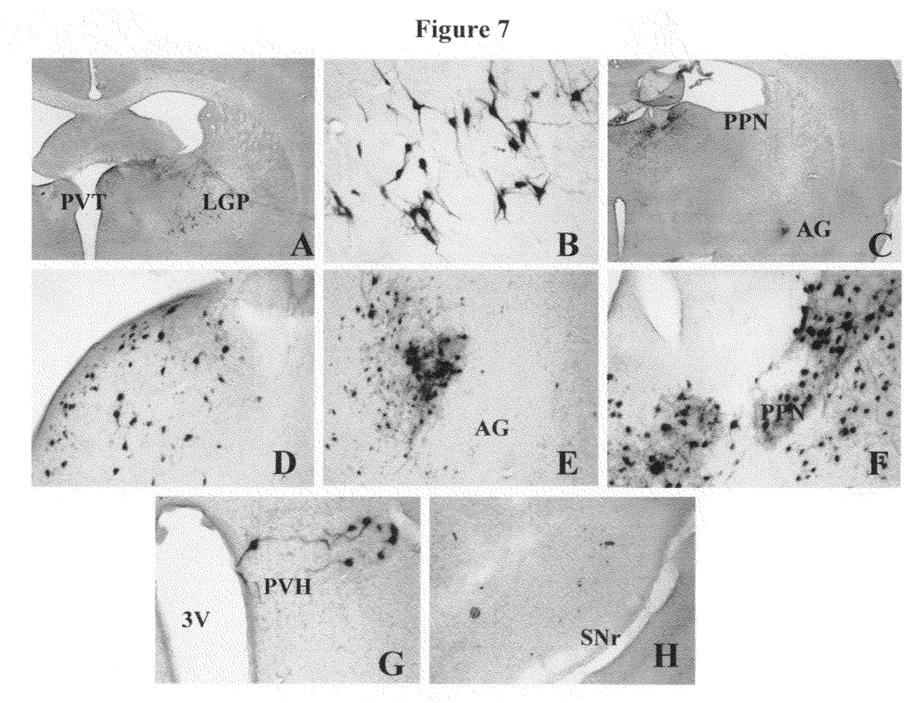

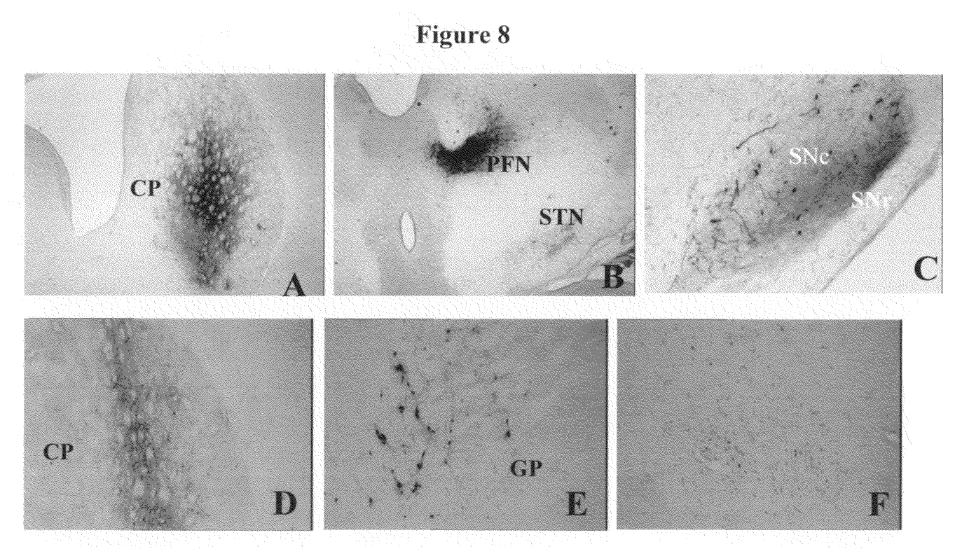

Transduction of rat striatum with Rabies-G pseudotyped EIAV-LacZ expressing vectors also gave efficient gene transfer to cells of both neuronal and glial phenotype within caudate putamen (FIGS. 16G and 16H). In addition, a far greater spread of transduced neurons was observed in regions caudal to the site of injection, including globus pallidus, thalamus, amygdala, ventral tegmental area (VTA), subthalamic nucleus (STN) and substantia nigra compacta (SNc) and reticulata (SNr) (FIGS. 6-8 and FIGS. 16G-16L). Anatomical connections are known to exist between these structures (see, for example, “Human Anatomy” 1976 Carpenter M. B. Williams and Wilkins Co. Baltimore, 7^{th} Edition, and references therein). Average transduction was seen anteroposteriously (7.5 mm anteposterior to the injection site) in 60×50 μm coronal sections spanning striatum, and also in neurons in 55×50 μm sections spanning GP and thalamus, and also in 40×50 μm sections spanning SN. This is the result of retrograde transport of viral vector to neurons in these areas from their axon terminals in striatum as well as anterograde transport of LacZ to neuron terminals whose cell bodies are in striatum. Cell counts indicated that 32650±1630 cells were transduced in striatum, while 14880±744 neurons were transduced in thalamus and 3050±150 neurons were transduced in substantia nigra. Staining in caudate putamen was paler and more punctate in comparison to VSVG vectors, with approximately 80% neurons and 20% glia transduced (FIGS. 16P-16U). Only glial cells appeared to be completely stained with LacZ. In comparison, neurons in other areas, such as GP, VTA and SNr, did stain in their entirety with LacZ (FIGS. 7 and 8).

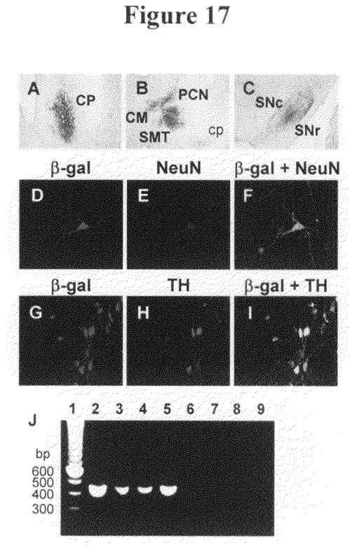

Confocal colocalization studies at the injection site indicate that the glia transduced were astrocytes. No projection neurons were transduced, in contrast with the VSV-G pseudotyped vectors. Anterograde transport of β-gal was also present in neurons transduced with the rabies-G pseudotyped vectors, as indicated by the pale staining of the thalamic reticular nucleus (from lateral globus pallidal neurons) and the substantia nigra pars reticulata (from striatal neurons) (FIGS. 16I and 16L). Confocal studies confirmed the neuronal nature of the cells transduced distally when rabies-G pseudotyped vectors were delivered into the caudate putamen, such as the NeuN positive pallidal neurons and the tyrosine hydroxylase positive dopaminergic neurons of the substantia nigra (FIGS. 17ii D-I).

Retrograde transport of viral vector itself was confirmed by PCR experiments using punches taken from thalamus and substantia nigra, since viral DNA in these areas could only be detected after rabies-G pseudotyped EIAV striatal transduction (FIG. 17iii). Control experiments where integrase mutant viral preparations or vector preparations, preheated at 50° C., were injected in the brain, failed to give any significant levels of transduction, thus excluding the possibility that pseudotransduction was responsible for the observed gene transfer (Hass et al (2000) Mol Ther 2, 71-80).

Long-term expression was observed after delivery of both types of vectors to the caudate putamen from 1 week for up to eight months post-injection in the present study. Expression of rabies-G pseudotyped vectors was observed both at the site of injection and at all the distal neurons that were transduced at one month post-injection (FIG. 17iA-C; only thalamus and substantia nigra are shown).

Comparison of Transduction Using EIAV Vectors Pseudotyped with VSVG and Rabies G to Substantia Nigra

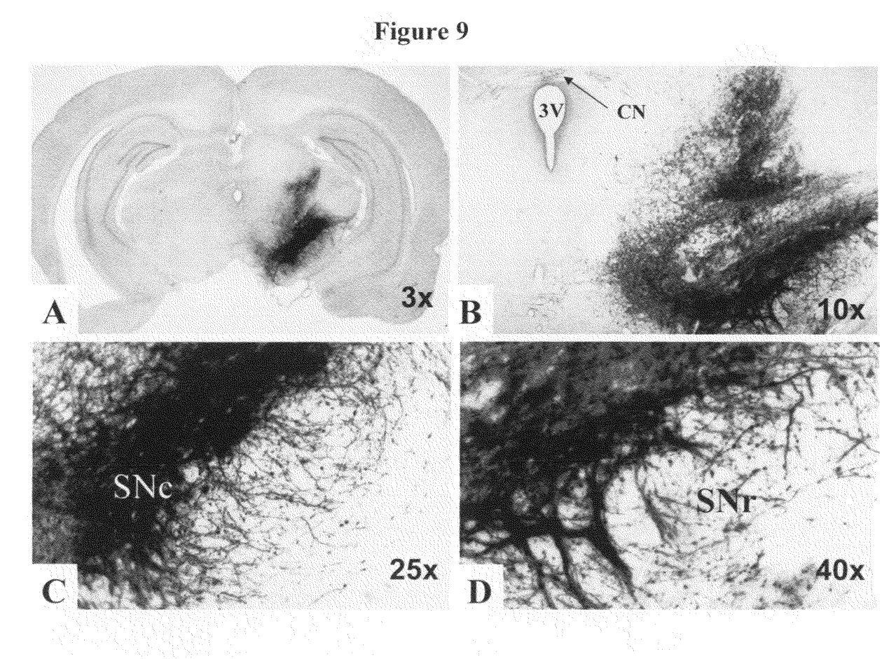

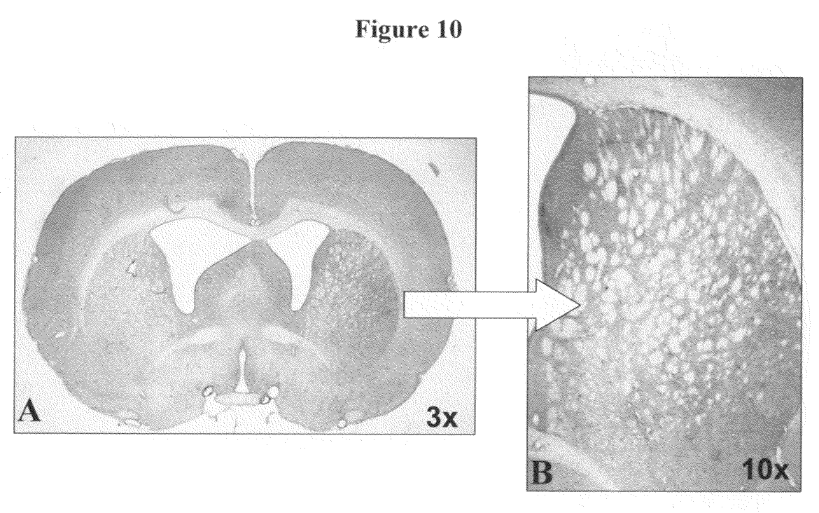

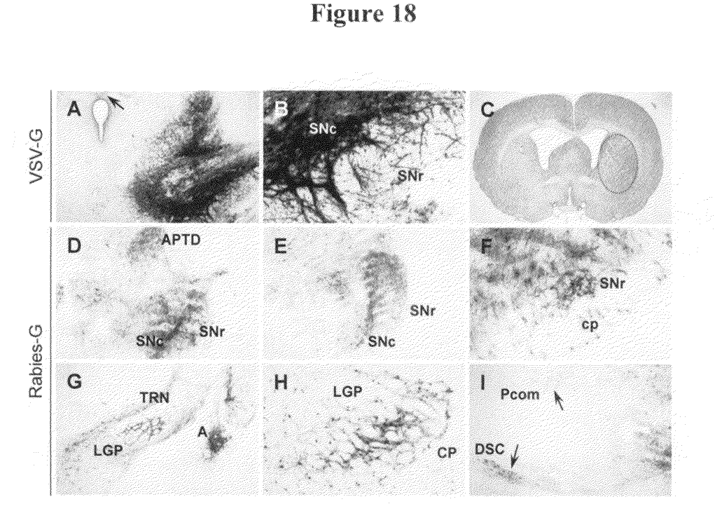

In order to compare the ability of the two different pseudotyped vectors to transduce central nervous system dopaminergic neurons, concentrated viral vector preparations were stereotactically injected in the vicinity of substantia nigra (medial lemniscus). Perinigral injections are preferable, since SN is prone to cell death after damage. VSVG pseudotyped EIAV-LacZ expressing vectors gave very efficient transduction of SNc and the thalamic structures caudal to it (FIG. 9, FIGS. 18A and 18B). LacZ was transported anterogradely to axon terminals of the nigrostriatal neurons, giving staining in the striatum (FIG. 10 and FIG. 18C). Projections of neurons from SNc to SNr were also stained. LacZ staining spanned 40×50 μm coronal thalamic/nigral sections.

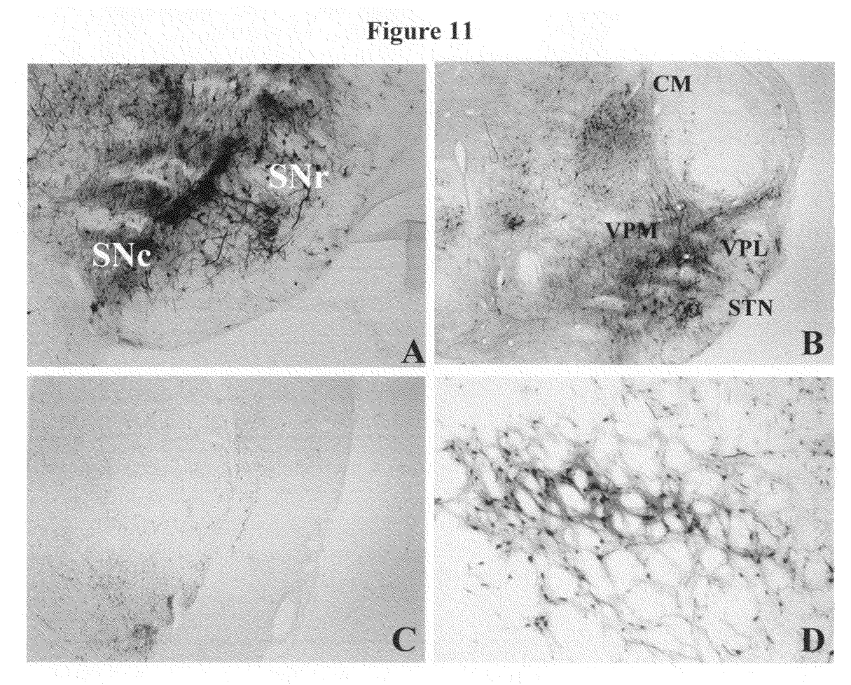

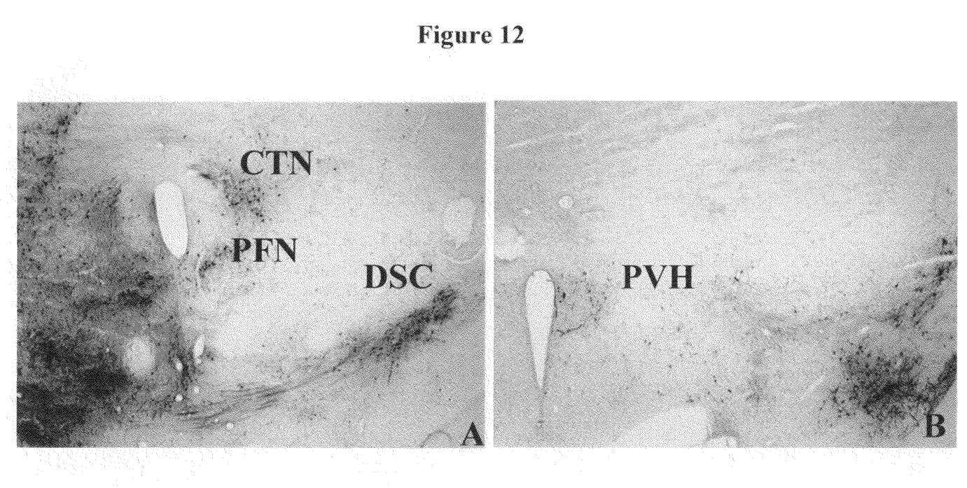

In contrast, perinigral injections of Rabies-G pseudotyped EIAV vector gave strong transduction of SNc neurons and much wider transduction of rostal thalamic nuclei, and in addition, transduction was observed in neurons of the SNr, STN, VTA, thalamus, GP and cortex (FIGS. 11,12). The β-gal staining was observed with the VSV-G pseudotyped vectors, and in addition, many fibres within the thalamus were stained. Transduction of distal neurons in the lateral globus pallidus and amygdala, where stronger β-gal staining was observed, was due to retrograde transport of virus from efferent connections to the substantia nigra pars reticulata and pars lateralis, respectively (FIGS. 18G and 18H). These neuronal projections from nigra were previously established by the retrograde tracer studies of Bunney and Aghajanian (Brain Res 117 234-435). In addition, on the contralateral side, transduction was observed of several (uninjected) commissural nuclei and their projections (FIGS. 12A and 18I), providing further evidence of retrograde transport operating with this vector.

### Example 3

### Isolation of Novel Trophic Factors

A VSV-G pseudotyped lentiviral vector system is constructed as described in Example 1, and used to express a cDNA library. A retroviral stock supernatant is produced by a transient method (as described above) and used to transduce primary rat ventral mesencephalic cultures established under low MOI, as described in Example 1. The expression of a secretable factor that acts as a trophic factor for dopaminergic neurons is determined in these cultures by measuring TH⁺ neurons per cm² on grids after 12 or 21 days culture in minimal media. (The trophic factor prevents naturally occurring apoptosis). In addition, changes in morphology of TH⁺ neurons are followed (such as more extensive neurite outgrowth and increased cell body size). Similar effects as observed with GDNF are used as a positive control.

### Example 4

### Isolation of Novel Neuroprotective/Survival Factors

A RbG pseudotyped lentiviral vector system is constructed as described in Example 1, and used to express a cDNA library under the control of a dopaminergic specific promoter. A retroviral stock supernatant is produced by a transient method (as described above) and used to transduce TH positive cells in primary rat ventral mesencephalic cultures, established as described in Example 1. The expression of a factor that acts as a survival/neuroprotective factor for dopaminergic neurons is determined in these cultures by measuring TH⁺ neurons per cm² on grids 12 days after exposure to 6-OHDA or MPP+. This identifies factors that act intracellularly and have an antiapoptotic effect. The contents of each of the surviving neurons are subsequently specifically amplified by putchclump PCR to determine the sequence of the introduced cDNA. In addition, the RNA from such cells is turned into cDNA, amplified by T7 RNA polymerase, and the aRNA hybridised to microarrays containing cDNAs obtained from differential display experiments (i.e. mRNAs preferentially expressed in dopaminergic neurons). This can also be applied on SN dopaminergic neurons in tissue sections using the technique of laser capture microdissection (Luo et al 1999, as above).

### Example 5

### Screening for Differentiation Factors for Neural Progenitor Cells

Neural progenitor cells are naturally occurring, and are the “new hope” for neural transplantation for brain injury and neurodegenerative disease. Human neural progenitors can be obtained commercially (Clonetics). These are neurospheres of subventricular origin that divide when exposed to EGF (originally identified and still worked upon by Canadian company NeuroSpheres). Rodent progenitor cells can also be isolated.

Several groups have tried to differentiate progenitors to dopaminergic neurons, but without great success (not one factor identified to date is capable of triggering the TH phenotype on its own). Recent papers demonstrate an unidentified astrocytic soluble factor involved in inducing dopaminergic TH+ phenotype in neural progenitors (Wagner et al (1999) Nat. Biotechnol. 17:653-659; Kawasaki et al (2000) Neuron 28:31-40). If such factor(s) are identified and can induce near 100% dopaminergic differentiation, they will prove very useful for differentiating grafts of neuroprogenitor cells into dopaminergic neurons after transplantation in the adult nervous system (where such inducible factor might not be expressed or expressed at low levels compared to the embryonic brain).

An RbG pseudotyped lentiviral vector system is constructed, as described in Example 1, and used to express a cDNA library from E14 embryo mesencephalon.

Dissection of E14 embryos yields mesencephalic cells. At day 3, when these cultures are stable, they are transduced with the retroviral library. Each 1×10⁵ primary mesencephalic cells are incubated with 0.5 ml of virus stock containing 10 μg/ml polybrene. This viral aliquot contains the equivalent of 200 transducing units (cDNAs). As this necessitates a large number of cultures (5000), the viral stock media needs to be appropriately diluted, frozen and used with sequential culture batches until the screening of the entire library is complete. After 8 hours, 0.5 ml of fresh growth medium is added to the culture and incubated overnight. The next day, the cultures are re-fed and allowed to continue until day 12, when the cells are stained for TH and counted. Where a significant increase in TH+ cell numbers is observed, genomic DNA is isolated, and cDNAs are amplified from small amounts (10 ng) of genomic DNA by PCR using retroviral vector primers, and sequenced. Chosen candidates are transfected into cells (293), and conditioned media is then used to re-confirm the result on fresh mesencephalic cultures, thus purifying the neurotrophic factor.

In an alternative approach, the library is transduced into HeLa cells, selected for antibiotic-resistance, and split into pools of 200 HeLa cells/cDNA clones (sub-libraries). The cells are subsequently co-cultured with the neurons, where they produce and secrete factors. Where an effect is seen, clones are selected and subjected to limit dilution clones, in order to isolate the cell of interest. The experiment is repeated with conditioned media from the single clone to further confirm the effect.

With low MOIs needed and efficiencies of only 20%, most cells will harbour only a single retrovirus, and only less than 10% of the cells might have multiple integrations (Onishi et al 1996).

Once a clone is isolated, it can be compared to GDNF (i.e. GDNF expressed from the same vector system) using a survival assay, or by measuring the extent to which it blocks the effect (for example, the apoptosis of TH+ neurons) of a neurotoxin (MPTP or 6-OHDA) on these cultures.

### Example 6

### Gene Transfer to Hippocampus Using VSV-G and Rabies-G Pseudotyped EIAV Vectors

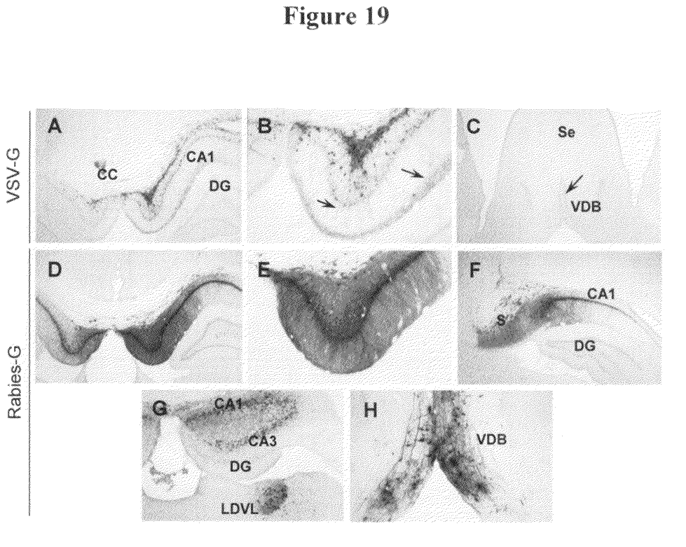

To test whether VSV-G and rabies-G pseudotyped EIAV vectors exhibit similar transduction properties to those observed when injected into the basal ganglia, these vectors were stereotactically injected into the right anteriodorsal hippocampus of rats. In the case of the VSV-G pseudotyped vectors, this led to strong transduction of neurons in the subiculum and, to a lesser extent, in the CA1 pyramidal cell layer (FIGS. 19A and 19B). Cells with neuronal morphology within the stratum oriens were also stained, while some glial transduction was observed within the corpus callosum. In addition, anterograde transport of β-gal was observed, resulting in weak staining of axon fibers projecting to stratum moleculare (FIG. 19B) and in few fibers projecting to septum (FIG. 19C).

By contrast, injections of rabies-G pseudotyped EIAV vectors into the hippocampal region led to strong β-gal staining of CA1 and CA3 pyramidal neurons within the stratum pyrimidale of the rostal hippocampus. This became restricted to the CA1 region in caudal aspects, and some staining was also observed in the CA4 pyramidal cell layer (FIGS. 19D-19F). Apical dendrites and axons of CA1 neurons were strongly stained. β-gal staining within the subiculum and corpus callosum was observed (FIG. 19F). Retrograde transport of the viral vector, and transduction of distal neurons projecting to the area of viral delivery, resulted in strong staining of the medial forebrain bundle nuclei in the lateral hypothalamus and in the vertical limb of the diagonal band of Broca (with axons projecting to the mediodorsal septal area and to the hippocampus via the fimbria of the formix) (FIG. 19H), supramammillary hypothalamic nuclei and thalamic nuclei (laterodorsal, anterodorsal and anteroventral nuclei) (FIG. 19G) (Segal (1974) Brain Res 78 1-15). Staining of the contralateral hippocampus was probably due to viral vector leakage during the injection along this folded structure, producing an identical but weaker pattern of staining on that side.

### Example 7

### Gene Transfer to Spinal Cord Using VS V-G or Rabies-G Pseudotyped EIAV Vectors

Methods

Intraspinal Injection

For intraspinal injection, anesthetized 2 month old rats were placed in a stereotaxic frame and their spinal cords were immobilized using a spinal adaptor (Stoelting Co., IL, USA). Injection was into the lumbar spinal cord, following laminectomy, with 1 μl of pONY8Z vector pseudotyped with rabies-G (n=3) or VSV-G (n=3) (6×10⁸ T.U./ml) at one site. Injections, controlled by an infusion pump (World Precision Instruments Inc., Sarasota, USA), were at 0.1 μl per minute through a 10 μl Hamilton syringe fitted with a 33 gauge needle. Following injection, the needle was left in place for 5 minutes before being retrieved. Two weeks following virus injection, rats received fluorogold (FG) administration. The sciatic nerve was exposed at mid-thigh level and cut 5 mm proximal to the nerve trifurcation. A small cup containing a 4% w/v fluorogold (FG) solution in saline was placed on the proximal segment of the transected nerve. Five days after application of FG the animals were perfused transcardially with 4% w/v paraformaldehyde. The lumbar spinal cord was dissected out and analysed by immunohistochemistry and X-gal reaction. The number of FG and β-gal double-labelled motoneurons was counted 3 weeks after injection of the viral vector. In addition, brains from these animals were also removed, and 50 μm coronal sections were stained in X-gal solution, as described above.

Intramuscular Injection

For intramuscular delivery, pONY8Z vectors were injected unilaterally in exposed gastrocnemius muscle with a microsyringe fitted with a 30-gauge needle (Hamilton, Switzerland). Two groups of rats were injected: the first group (n=3) received pONY8Z pseudotyped with rabies-G, and the second group of rats (n=3) received pONY8Z pseudotyped with VSV-G (titer of both type of vectors is 3×10⁸ T.U./ml). Five sites per animal were injected with 10 μl per site. The solution was infused at speed of approximately 1 μl/min. Two animals from each group were sacrificed 3 weeks post injection. The remaining two rats were anesthetized by an intraperitoneal injection of Hypnorm/Hypnovel solution, and FG administration was performed as described above. Two days after application of FG, the animals were sacrificed. All animals were perfused transcardially with 4% w/v paraformaldehyde. Subsequently, the muscles were excised and snap frozen in liquid nitrogen. Spinal cords were excised and cryoprotected in 30% w/v sucrose for 2 days. Transverse and longitudinal sections (25 μm each) of both the muscle and spinal cords were analysed by immunohistochemistry and X-gal reaction. To evaluate the number of transduced neurons, motoneurons, lumbar and thoracic spinal cord were analyzed. The number of β-gal-positive cells double-labelled with NeuN were examined in every third section. The proportion of infected motoneurons was expressed as the percentage of fluorogold back-labeled cells expressing β-gal.

Results

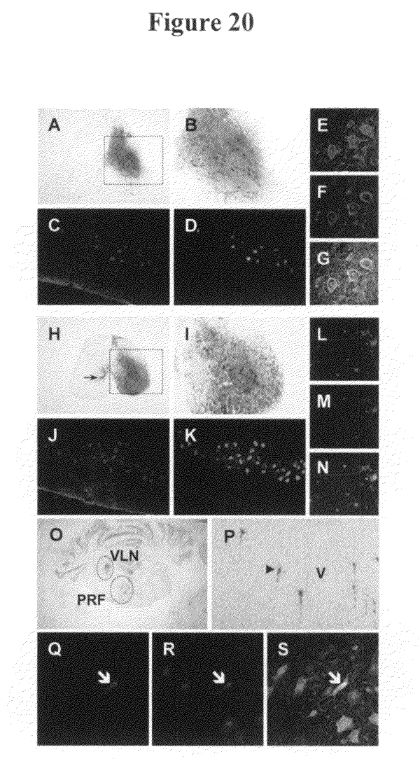

To determine the transduction efficiency of the EIAV vector, intraspinal and intramuscular injections of the β-gal-expressing vectors were performed in the rat. Intraspinal injection of the lentiviral vector was associated only with a mild degree of inflammation, with no significant cell damage. All rats tolerated the surgery and lentiviral vector injections without complication. Moreover, coordination and movement of operated animals was unaffected, indicating the absence of functional deterioration following intraspinal injection of the viral vector. Examination of transverse sections of the spinal cord revealed robust reporter gene expression after delivery of both VSV-G and the rabies-G pseudotyped lentiviral vectors (FIGS. 20A, 20B, 20H and 20I). Injection in the lumbar spinal cord led to β-gal expression in 10,260±513 and in 16,695±835 cells with VSV-G and rabies-G pseudotyped vectors, respectively. The rabies-G pseudotyped lentiviral vectors produced a more extensive rostrocaudal spread of expressing cells within the area of viral delivery (lumbar spinal cord) and also in the adjoining thoracic spinal cord.

To identify the phenotype of the cells transduced after intraspinal injections, sections were double-labelled with antibodies to β-gal and either NeuN or GFAP. On average, 90% and 80% of the transduced cells were double-labelled with NeuN after VSV-G and rabies-G pseudotyped vector delivery, respectively (FIGS. 20E-20G and 20L-20N). To assess the percentage of motoneurons expressing the reporter gene, motoneurons were back-labelled with FG (FIGS. 20C, 20D, 20J and 20K). The number of FG-positive motoneurons expressing β-gal was evaluated in longitudinal sections of the lumbar spinal cord. Analysis of these sections showed that 52% and 67% of the FG-back labeled motoneurons expressed β-gal after intraspinal injections of VSV-G and rabies-G pseudotyped EIAV vectors, respectively.

Interestingly, brainstem motoneurons of the tectospinal, vestibulospinal and reticulospinal tracts, as well as corticospinal motoneurons located in the layer V of primary motor cortex, were retrogradely transduced following intraspinal injection only of the rabies-G lentiviral pseudotyped vector (FIGS. 20O and 20P). Some spinal commissural interneurons projecting from the contralateral side were also retrogradely transduced (FIG. 20H). Interestingly, retrograde transport of the rabies pseudotyped vector was also found in lumbar spinal motoneurons following injection into the gastrocnemius muscle (FIGS. 20Q-20S). Intramuscular injections of rabies-G pseudotyped lentiviral vector led to β-gal expression in 27% of the FG-back labelled motoneurons (approximately 850±90 transduced motoneurons). No muscle transduction was observed with this vector. By contrast, the VSV-G pseudotyped vector transduced muscle cells surrounding the injection site at low efficiency, but did not label any cells in the spinal cord.

### Example 8

### Minimal Immune Response in CNS After EIAV Vector Injection. Methods

Investigation of the Immune Response

Groups of rats received intrastriatal injections of pONY8Z vector pseudotyped either with VSV-G (n=6) or rabies-G (n=6) or an equivalent amount of PBS, using the stereotactic procedure described above. Following euthanasia at 7, 14, and 35 days post injection, brains were removed, snap frozen directly in OCT and analysed. Sections (15 μm) were cut onto APES (Sigma) coated slides using a Leica CM3500 cryostat (Milton Keynes, UK). One in every 10 sections was stained with X-gal for 3 hours at 37° C. to identify areas of gene transfer. Adjacent sections were selected and stained with monoclonal antibody tissue culture supernatant (TCS) against OX1 (leucocyte common antigen), OX18 (MHC class I), OX42 (complement receptor type 3 on microglia and macrophages) and OX62 (dendritic cells). These antibodies were a kind gift from the MRC Cellular Immunology Unit, Sir William Dunn School of Pathology, Oxford. Sections were incubated overnight in neat TCS and following several washes in PBS, incubated for 1 hour with an HRP conjugated rabbit anti-mouse antibody (Dako, UK). Positive staining was then visualised to a brown color using a diaminobenzidine (DAB) kit (Vector Labs, USA). Sections were counterstained with hematoxylin, dehydrated, cleared and mounted using DePeX (BDH Merck, Poole, UK). X-gal stained sections were counterstained using carminic acid (Sigma, UK) and mounted using Permount (Fisher, USA).

Results

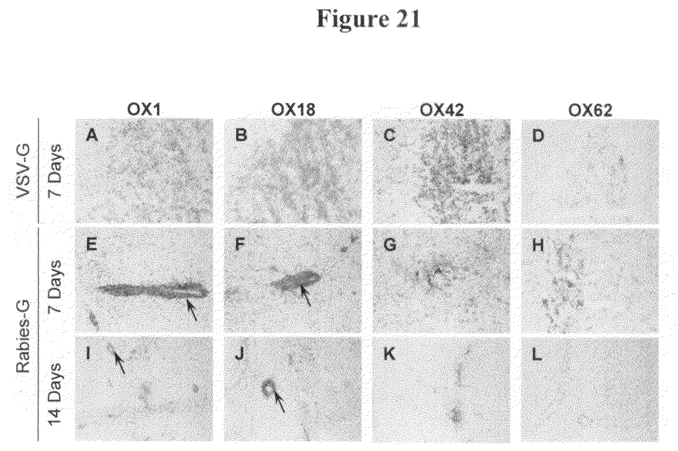

At different time points after gene transfer to the brain (striatum), specific antibody markers were used to detect immune responsive cells at the site of injection, at different time points after vector delivery. In no cases after stereotactic delivery was any adverse brain pathology observed. Control injections with PBS caused a negligible immune reaction that consisted of a small infiltration of OX-42⁺/ED1⁺ activated macrophages/microglia around the needle tract in the cortex and striatum, and also along white matter tracts, such as corpus callosum. No staining was observed with any of the other markers when PBS was injected. This immunoreactivity declined, but was still detectable at 35 days. A similar response with these markers was observed with both viral vector preparations, and probably represents the reaction to the injection procedure. In addition, the VSV-G pseudotyped vectors resulted in an infiltration of OX18⁺, MHC class I positive cells in the ipsilateral striatum, present at all time points, but no leucocytes or dendritic cells were observed at any time point (FIGS. 21A-21D). However, the rabies-G vector injection initiated a more acute immune response with infiltrating leucocytes, dendritic cells and MHC class I immunopositive cells into striatum and cortex, and also along white matter tracts, meninges and subventicular cell layers (FIGS. 21E-21H). Some perivascular cuffing and tightly packed inflammatory cells were observed within the striatum with the OX1 and OX18 markers (FIGS. 21E and 21F). Reduced levels of response, including the absence of dendritic cells, were detected at 14 days, and declined to background levels by 35 days.

### Example 9

### Gene Transfer into the Sensory Nervous System

Methods

Injection of the Virus into the Dorsal Horn of the Spinal Cord

The intraspinal injection described in Example 7 was followed, except that the site of injection was in the dorsal horn instead of in the ventral horn. A group of rats was injected with pONY8Z or pONY8.1Z (rabies-G or VSV-G), or an equivalent amount of PBS, via a posterior laminectomy within the dorsal horn of the spinal cord. Three injection sites at the lumbar level, separated by 2 mm, were performed. Each rat received 1 μl per site of the viral solution at dorso-ventral coordinate of 0.5 mm. PONY8.1Z (VSV-G) was obtained directly from pONY8.0Z by digestion with SalI and partial digestion with SapI. Following restriction, the overhanging termini of the DNA were made blunt ended by treatment with T4 DNA polymerase. The resulting DNA was then re-ligated. This manipulation resulted in a deletion of sequence between the LacZ reporter gene and just upstream of the 3′PPT. The 3′ border of the deletion was nt 7895 with respect to wild type EIAV, Acc. No. U01866. Thus pONY8.1Z does not contain sequences corresponding to the EIAV RREs.

Direct Injection of the Virus in the Dorsal Root Ganglia

Dorsal root ganglia (DRG) were surgically exposed by dissecting the musculus multifidus and the musculus longissimus lumborum, and by removing the processus accessorius and parts of the processus transversus. EIAV vectors (pONY8 or pONY8.1 version) coding for the reporter gene β-gal were injected directly in the DRG. Subjects received 0.5 μl of the viral solution per ganglion. All injections were done by using a stereotaxic frame and a Hamilton syringe with 33-gauge needle. The solution was slowly infused at the speed of approximately 0.1 μl/min.

Peripheral Administration of the Virus

The procedure of the application of the virus on the skin surface was described in Wilson et al. (1999). Briefly, the hair was removed from the dorsal of the hindfoot surface. The skin was scarified by using medium-coarse sandpaper. Ten microliters of the viral solution was applied to each foot. The side of pipettor tip was used to spread the virus. The virus was retrogradely transported to the DRG. Subcutaneous injections of the virus in the hindfoot were also performed. Each rat received unilateral application or injection of 10 μl viral solution.

Direct Injection of the Virus into the Sciatic Nerve

For intranerval injection, the right sciatic nerve of an anaesthetized rat was surgically exposed. The nerve was gently placed on to a metal plate, and pONY8Z or pONY8.1Z pseudotyped with VSV-G or Rabies-G was injected with a 33-gauge Hamilton syringe over 3 min. The volume injected per rat was 1 μl. The sciatic nerve was anatomically repositioned, and the skin was closed with vicryl 5/0 sutures.

Results

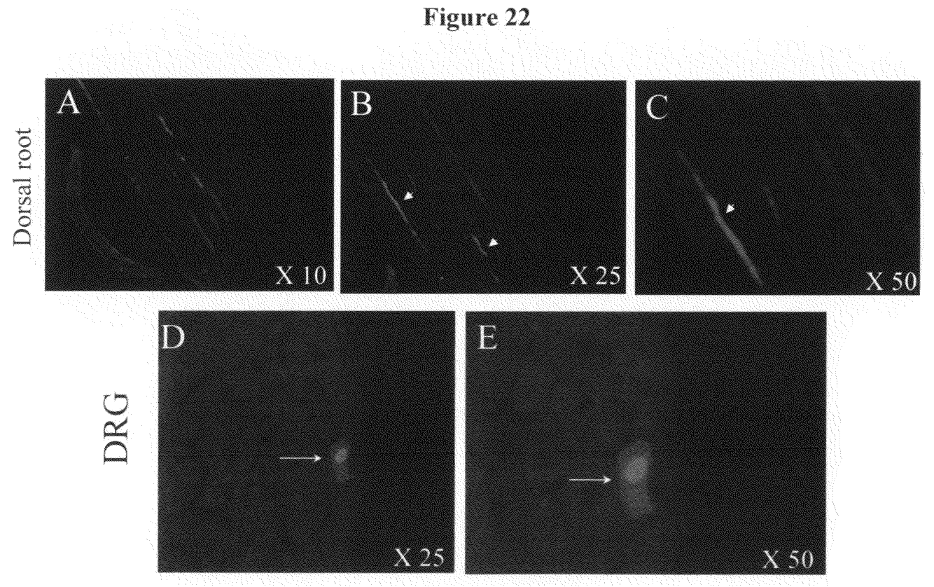

pONY8Z vectors were injected into the dorsal horn in four rats and analysed 5 weeks post-transduction (rabies-G 3.8×10⁸ TU/ml, n=2; VSV-G 1.2×10⁹ TU/ml, n=2). Histological sections from the spinal cord, the dorsal root, and the DRG were examined at various magnifications. All animals showed expression of the marker gene in the immediate neighborhood of the site of injection into the spinal cord. Of three rats injected into the spinal cord with pONY8Z rabies-G, two showed expression of β-gal in Schwann cells. Axonal expression was also seen (FIGS. 22A-22C). The two rats displayed retrogradely transduced DRG neurons (FIGS. 22D and 22E). However, in contrast to pONY8Z rabies-G injected rats, no β-gal reactivity was detectable in dorsal root and DRG sections from rats injected with pONY8Z VSV-G.

### Example 10

### Injection of EIAV Pseudotyped with Rabies-G or VSV-G Envelopes into the Cerebrospinal Fluid (CSF) and Treatment of MS Using an Intrathecal Route for Gene Therapy

Mutant Rabies G

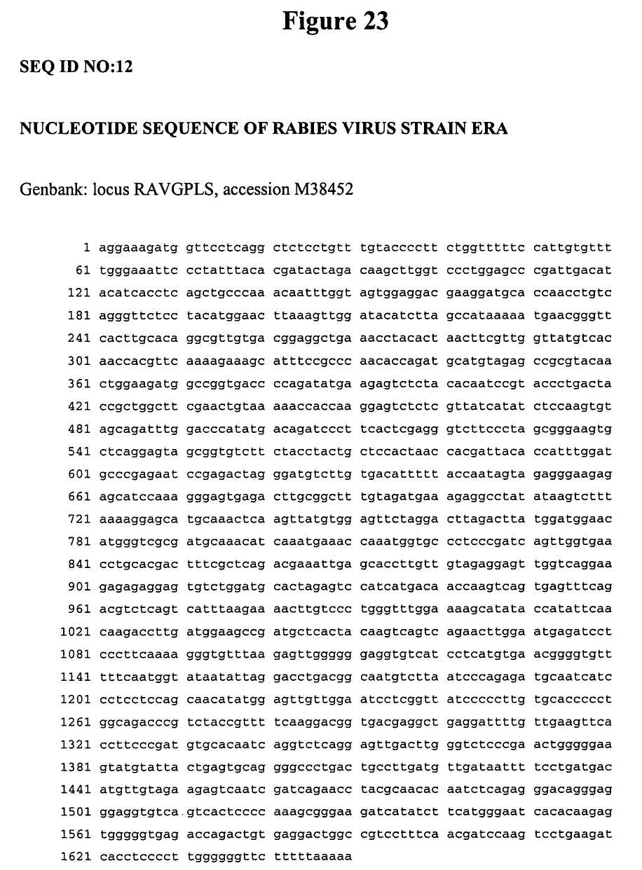

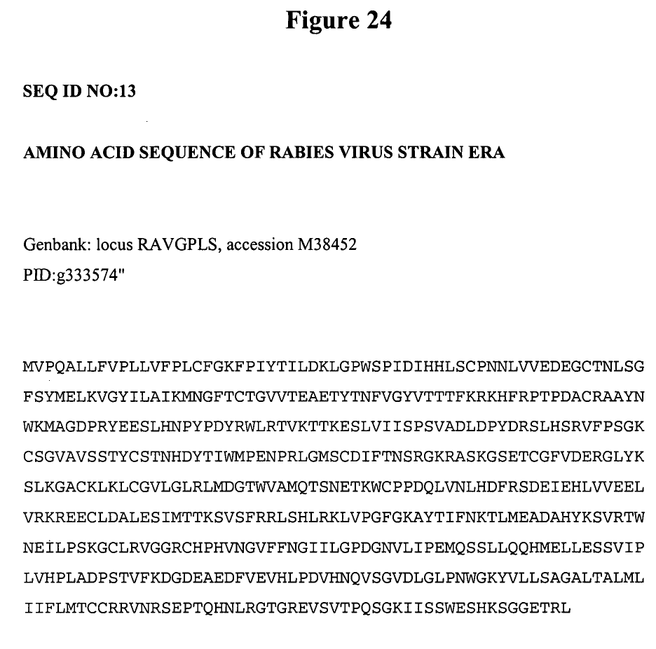

EIAV vectors were pseudotyped with wild-type and 2 variants of the ERA strain of rabies-G envelopes. The sequence of rabies virus strain ERA is shown in FIGS. 23 and 24 (SEQ ID NOs:12 and 13). A single mutant of the wild-type ERA strain (ERAwt) was generated by replacing arginine at amino acid 333 with glutamine. This mutant, which is naturally occurring and apathogenic in adult mice, was termed ERAsm. An additional substitution at amino acid 330, from K to N, resulted in a double mutant of ERAwt, named ERAdm. Both these envelopes were used to pseudotype the EIAV vectors expressing a marker gene LacZ.

In more detail, a partial PCR fragment of the ERAwt that incorporated the 2 amino acid changes was amplified using the following primers:

(SEQ ID NO: 16)

(5′ to 3′)
CTA CAA CTC AGT CAT GAC TTG GAA TGA GAT CCT CCC
CTC AAA AGG GTG TTT AAG AGT TGG GGG GAG G

(SEQ ID NO: 17)

(5′ to 3′)
CCT TTT GAG GGG AGG ATC TCA TTC CAA GTC ATG ACT
GAG TTG TAG TGA GCA TCG GCT TCC ATC AAG GTC

The full-length fragment of the ERAdm (incorporating the 2 amino acid changes) was then amplified using the following primers:

| | |
|---|---|
| (5′ to 3′) | |
| ACC GTC CTT GAC ACG AAG CT | (SEQ ID NO: 18) |
| (5′ to 3′) | |
| GGG GGA GGT GTG GGA GGT TT | (SEQ ID NO: 19) |

The resulting fragment was cloned into pSA91 using appropriate restriction enzymes. Successful clones were sequenced and used to produce EIAV vectors using the transient transfection method.

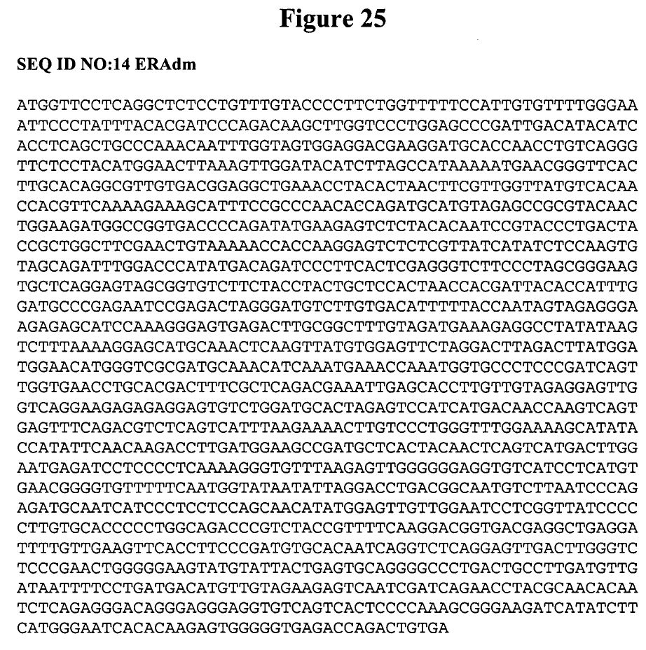

The sequence of the ERAdm is shown in FIG. 25 (SEQ ID NO:14).

CVS

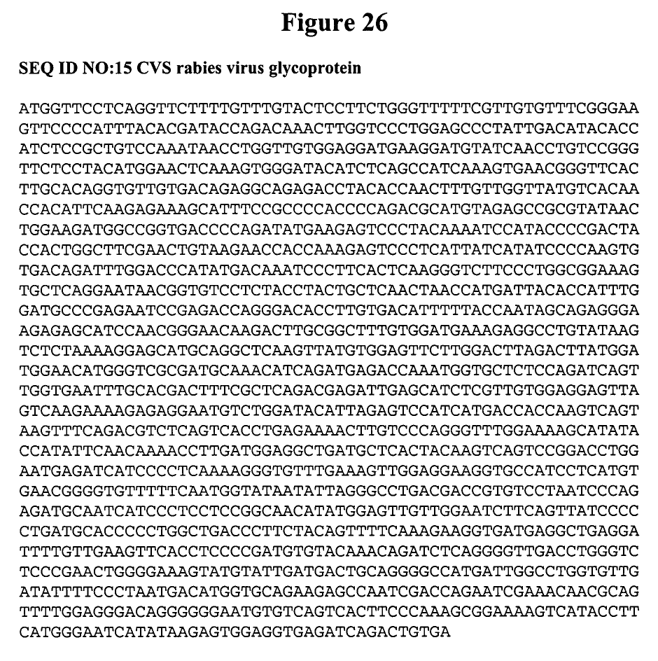

cDNA for CVS (Challenge Virus Standard) rabies virus glycoprotein was obtained from ATCC (ATCC number 40280 designation pKB3-JE-13). The fragment containing the complete coding sequence of the glycoprotein was excised using EcoRI, cloned into pSA91 and sequenced (Bk 1092 pg 75). The sequence is shown in FIG. 26 (SEQ ID NO:15).

Viral Transductions

The titres of the various pseudotyped EIAV vectors, as determined by transduction efficiencies in D17 cells, were as follows:

| | |
|---|---|
| pONY8Z ERAwt | 7 × 10⁸ TU/ml |
| pONY8Z ERAsm | 9 × 10⁸ TU/ml |
| pONY8Z ERAdm | 1 × 10⁸ TU/ml |
| pONY8Z CVS | 7 × 10⁸ TU/ml |

Stereotaxic administrations were performed under Hypnorm & Hypnovel anesthesia using a 5 μl Hamilton syringe with a 33-gauge blunt tip needle. A total of 8 rats received 10 μl injections of viral vectors into the CSF at coordinates: AP=−0.92; L=1.4; V=3.3. The first group of animals (n=4) were injected with EIAV pseudotyped with VSV-G envelope. In the second group (n=4) all the viral vectors were rabies-G pseudotyped. The viral titre was 7×10⁸ TU/ml. The lentiviral solution was slowly infused at the speed of 0.2 μl/minute using an infusion pump (World Precision Instruments Inc.). After viral vector injections, the skin was closed using a 5-0 Vicryl running suture and following surgery, animals were kept warm until recovery was complete. All surgical procedures were approved by the local veterinarian and ethical committee and were carried out according to Home Office regulations.

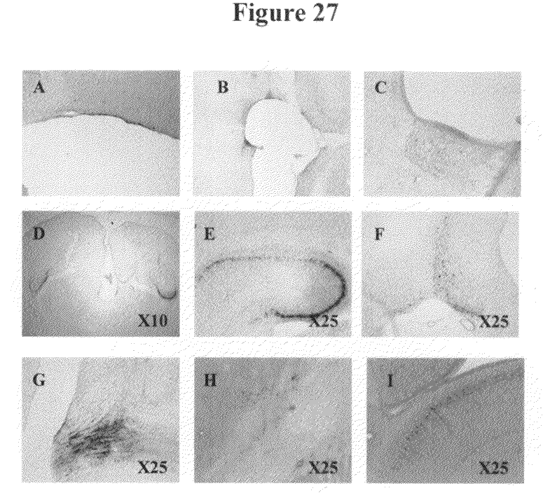

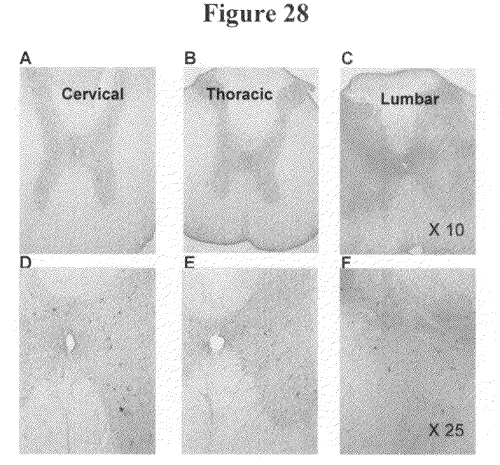

Following injections into the CSF, the expression of the marker gene LacZ could be demonstrated in different areas of the brain and spinal cord (FIG. 27). The rabies-G pseudotyped vectors were able to infect the ependymal and leptomeningeal cells (FIGS. 27A-27C). Strong bilateral transduction was also observed in the hippocampus (mainly in CA3), corpus collasum, and septum (FIGS. 27D-27I). The virus also spread to the spinal cord (FIGS. 28A-28F).

In contrast, no signs of transport or biodistribution were seen with VSV-G pseudotyping.

As demonstrated by these results, the present invention may represent an alternative treatment for inflammatory neurological disorders. Lentiviral-mediated delivery of cytokines-encoding genes to the CSF in accordance with the present invention shows the following major advantages: i) the availability of high cytokine levels widely in the CNS; ii) long-term and persistent expression of exogenous genes after incorporation into the DNA of the host cell; and iii) absence of the immune response to the viral particle.

### Example 11

### Gene Transfer to Muscle in Neonatal Mice Using EIAV-Rabies-G and EIAV-CVS

To determine if EIAV vectors pseudotyped with rabies-G or CVS envelope is retrogradely transported to the mouse spinal cord, P6 neonatal mice received intramuscular injection of pONY8Z rabies-G viral stock solution (titre 5.7×10⁸ TU/ml). Seven mice were injected with pONY8Z rabies-G (10 μl, n=2; 20 μl, n=2; 30 μl, n=3). The second group of mice were injected with pONY8Z CVS (titre 7×10⁸ TU/ml, n=3, volume injected=30 μl).

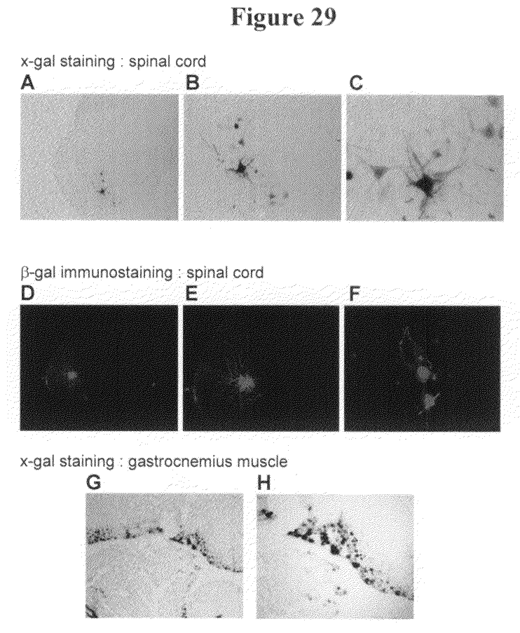

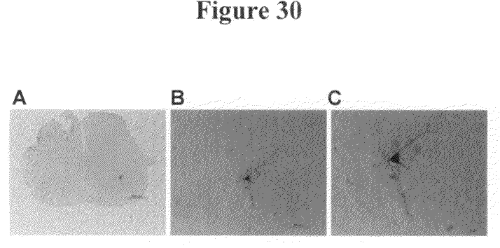

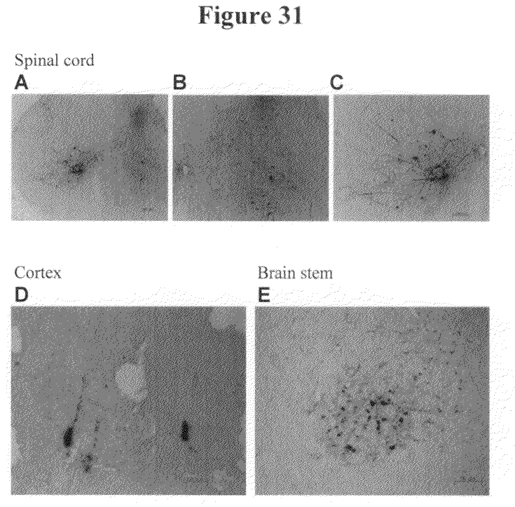

The results are shown in FIGS. 29 and 30, and the experiment demonstrates that a large number of motor neurons (MN) were retrogradely transduced after injection of the viral particles in the gastrocnemius muscle. In the present study, 10-12 MN (˜50% of MN) per section were X-gal-positive in pONY8Z-rabies-G injected mice (FIG. 29). In EIAV-CVS injected animals, 7-8 MN per section were x-gal positive (FIG. 30). Transduced cells were found to be localised in the ventral horn and only on one side. Examination of the morphology of transduced cells suggested that these cells were motorneurons (cells with large size). β-gal immunostaining was also performed. Muscle cells were also transduced in EIAV-rabies-G injected animals (FIG. 29).

### Example 12

### Gene Transfer to Rat Spinal Cord Using EIAV-CVS

For intraspinal injection, anesthetized 2 month old rats were placed in a stereotaxic frame and their spinal cords were immobilized using a spinal adaptor (Stoelting Co., IL, USA). Injection into the lumbar spinal cord following laminectomy with 1 μl of pONY8.0Z vector pseudotyped with CVS (n=3) (7×10⁸ T.U./ml) at one site. Injections, controlled by an infusion pump (World Precision Instruments Inc., Sarasota, USA), were at 0.1 μl per minute through a 10 μl Hamilton syringe fitted with a 33 gauge needle. Following injection, the needle was left in place for 5 minutes before being retrieved. Four weeks after viral injection animals were perfused transcardially with 4% w/v paraformaldehyde. The spinal cord and brain were dissected out and analysed X-gal reaction.

The results from this experiment are described in FIG. 31. Injection of EIAV-Lac CVS into the spinal cord induced strong transduction in the injected side, with retrograde transport to the contralateral side of the spinal cord. Interestingly motor neurons in the brain stem and cortex were transduced by retrograde transport (FIG. 31).

### Example 13

### Injection of EIAV Vectors Pseudotyped with CVS Envelope into the Striatum

Approximately 2×10⁶ TU of each vector was slowly infused into the striatum of adult male Wistar rats (300 g) using the stereotaxic coordinates AP 0 mm, ML 3.5 mm, DV 4.75 mm, and left for 2 or 4 weeks. The rats were then sacrificed and transcardially perfused with 4% paraformaldehyde. Following an overnight incubation in 4% paraformaldehyde, the brains were cryoprotected in 30% sucrose for at least 3 days, after which they were frozen and cut into 40 μm coronal sections. X-gal staining and immunohistochemistry were performed.

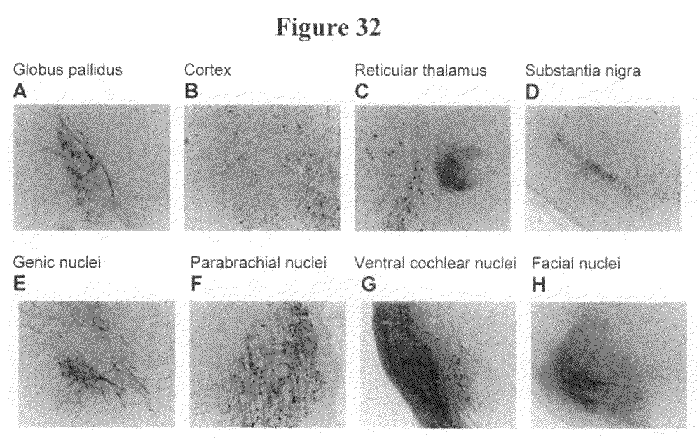

As shown in FIG. 32, when EIAV vectors pseudotyped with the CVS envelope was injected into the striatum, there was strong expression in the globus pallidus. Retrograde transport was observed in the cortex, various thalamic nuclei, amygdala, hypothalamus, supraoptic nucleus, deep mesencephalic nuclei and substantia nigra. In addition, retrograde transport to the caudal regions of the brainstem was observed. In this region, various nuclei such as the nuclei of the brachium inferior colliculus, paraleminiscal nuclei, genic nuclei, parabrachial nuclei, ventral cochlear nuclei and facial nuclei were positive for X-gal staining.

### Example 14

### Retrograde Transport to the Brain Following Subretinal Delivery of a Lentivirus Vector Pseudotyped with the Rabies Envelope

Methods

The transient three plasmid transfection method was used to generate an EIAV virus vector based on the pONY8.0 CMV GFP genome pseudotyped with the Rabies envelope (pSA91ERAwt). The virus (batch number OBM039) was titered biologically and estimated to be 1×10e10 TU/ml. A total of 4 ul (2×2 ul) was sub-retinally injected into C57/bl-6J mice and tissues were harvested at different time points for analysis of gene expression.

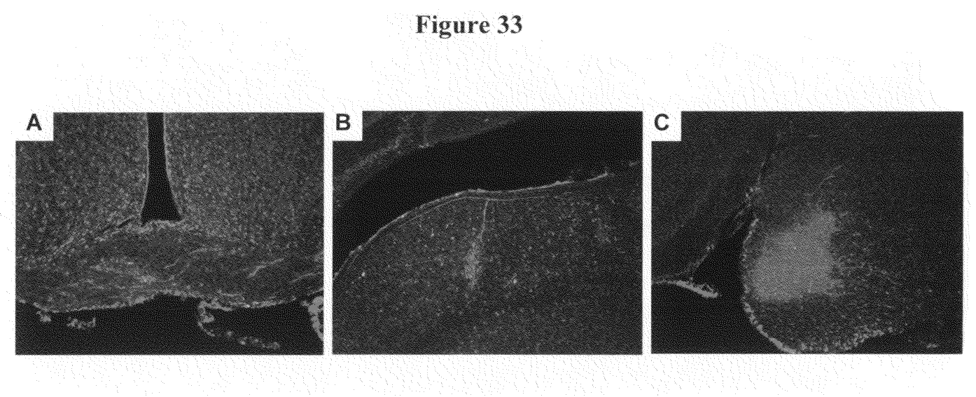

This demonstrates that sub-retinal delivery of this Rabies pseudotyped EIAV vector leads to retrograde transport of the vector along the optic nerve to the optic chiasm at the base of the brain, and from there, travels along the optic tract to the region of the lateral geniculate nuclei (LGN), a subdivision of the subcortical thalamus (FIG. 33).

The optic nerve fibres from each eye cross over in a very specific way at the optic chiasm—fibres originating in the nasal part of the retina cross over to the opposite hemisphere, while those originating in the temporal retina do not, but continue to the same side of the brain. Therefore, sub-retinal delivery to a single eye can lead to retrograde transport to both cerebral hemispheres. Alternatively, if the sub-retinal injection is restricted to a particular region of the eye, either nasal or temporal, then a single cerebral hemisphere may be targeted.

### Example 15

### In Vitro Validation of SMA Fibroblast

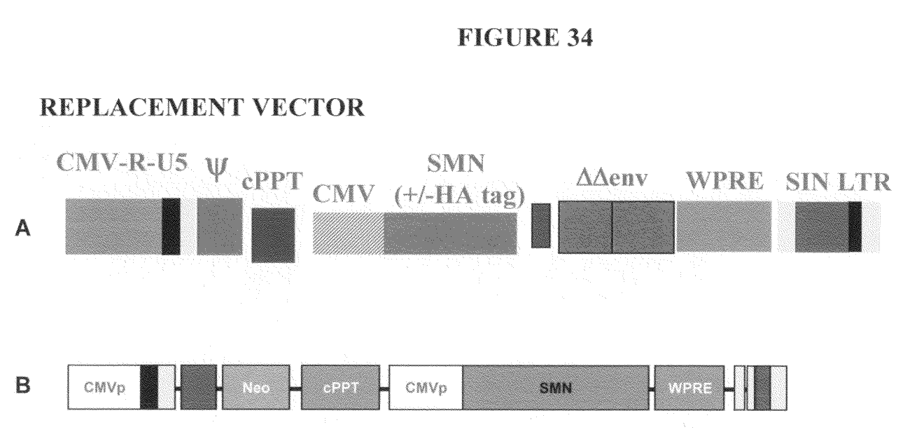

Construction of Smart2SMN and pONY 8.7NCSMN vectors, as shown in FIG. 34, is described by Mazarakis et al. (2001). SMN gene was a gift from Dr. Arthur Burghes (Ohio State University, Ohio, USA). The Smart2SMN vector was pseudotyped with rabies-G envelope protein derived from ERA strain.

SMA fibroblasts represent an in vitro model of SMA, and primary fibroblast cultures were established from SMA patients, type I, according to standard methods (DiDonato et al., 2003; Human Gene Therapy 14:179). These cells show very low or no expression of SMN protein. The Smart2SMN vector pseudotyped with rabies-G envelope was used to transduce SMA fibroblast at an MOI of 50 and 100, essentially as described in Mazarakis et al. (Human Molecular Genetics, 2001).

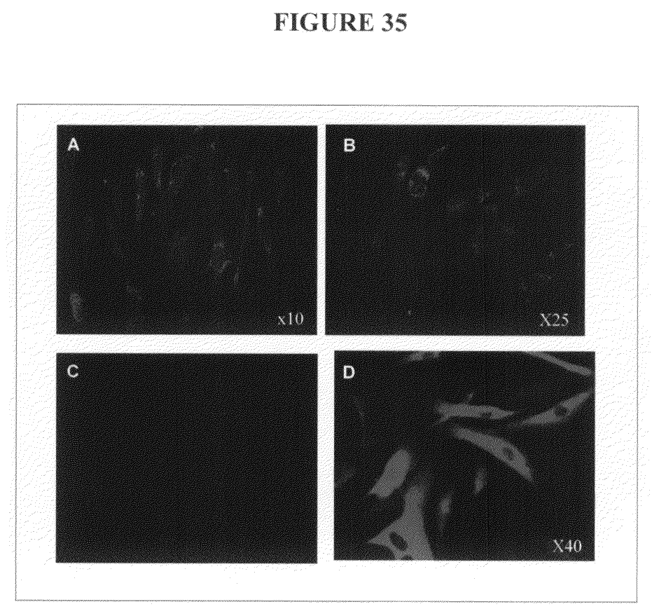

A Smart2LacZ ERAwt transduction and untransduced cells were used as negative controls. Immunocytochemistry was used to confirm expression of the SMN protein from pSMT2SMN ERAwt. Confocal microscopy demonstrated strong positive SMN staining in the cytoplasm. This experiment also demonstrates the use of EIAV to restore gems in the nucleus of SMA fibroblast (FIG. 35). The best results were obtained with an MOI 100. No such staining was seen in the negative controls.

Cell counting showed an average of 8 gems per SMN transduced cell. An average of 3-6 nuclear gems was seen in treated fibroblasts from SMA patients by Skordis et al. (PNAS 100, 4114-4119) and DiDonato et al. (Hum Gen Ther 14, 179-188).

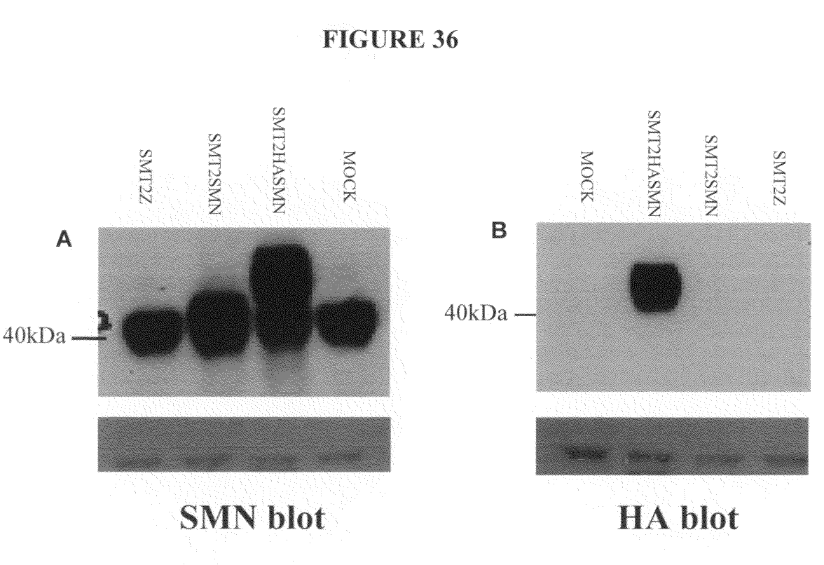

To test the efficiency of the SMN vectors, the dog osteosarcoma cell line, D17 was used. FIG. 36 shows a Western Blot, using SMN antibody (Transduction Laboratories) recognising SMN, and antibodies against HA tag, which demonstrates expression of SMN in those cells transduced with the SMN vector.

Although D17 cells express some SMN protein, overexpression was seen when cells were transduced with SMN vectors, compared to control cells transduced with LacZ vector. The expression of SMN transgene was confirmed using HA tag antibody.

### Example 16

### SMN Gene Replacement in an SMA Animal Model

SMN-1 gene replacement strategy using gene therapy can be used for rescuing motor neurons from cell death in an animal model of SMA and in SMA patients.

Mouse Model of Type III SMA

Type III mice display muscle weakness, motor neuron degeneration and a reduction in SMN protein level (an average of 4.5 nuclear gems were counted per motor neuron in the type III SMA mice versus 9.8 nuclear gems in the age-matched control).

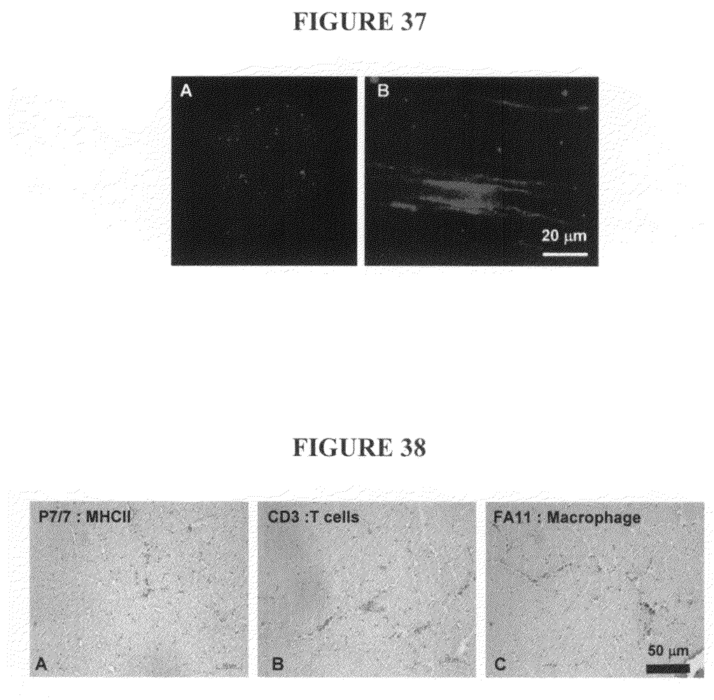

Four type III animals received unilateral injections of Smart2SMNHA into leg muscles. Mice were perfused with 4% paraformaldehyde and spinal cord was extracted and stored at −80° C. Expression of SMN in motor neurons was monitored using HA and SMN antibodies. Confocal microscopy demonstrated efficient transduction of motor neurons by retrograde transport, as demonstrated by HA tag immunostaining (FIG. 37A). Further analysis demonstrated good SMN gene transfer into muscle in these mice (FIG. 37B).

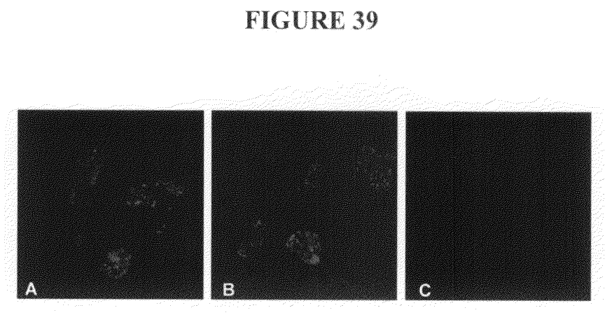

SMN gene transfer in mouse model of type III induced minimal immune response is shown in FIG. 38.

Mouse Model of Type I SMA.

The animal model of type I SMA represents a model of the severe form of SMA. These mice display motor neuron death, muscle weakness, and die by postnatal day 14. The aim of this work was to extend mouse survival using muscle delivery of LentiVector® expressing SMN gene.

Neonatal SMN mice of age 1-2 days were used in this study. Neonate injections were performed as follows: animals were briefly anaesthetized in hypothermia, and viral vectors were injected using a Hamilton microsyringe fitted with a 33 gauge needle. The following groups are included in the present study.

Smart2-SMN group (n=8)

Leg muscles: 20 μl each

Intraperitoneal: 10 μl

Diaphragm muscle: 10 μl

Face muscles: 20 μl

Tongue: 10 μl

Intracranial (brainstem): 5 μl

Muscles of the thoracic trunk: 10 μl

Smart2-GDNF group (n=4)

Leg muscles: 20 μl each

Intraperitoneal: 10 μl

Diaphragm muscle: 10 μl

Face muscles: 20 μl

Tongue: 10 μl

Intracranial (brainstem): 5 μl

Muscles of the thoracic trunk: 10 μl

Smart2-SMN+Smart2-GDNF group (n=6)

Leg muscles: 20 μl each

Intraperitoneal: 10 μl

Diaphragm muscle: 10 μl

Face muscles: 20 μl

Tongue: 10 μl

Intracranial (brainstem): 5 μl

Muscles of the thoracic trunk: 10 μl

Smart2-LacZ group (n=6)

Leg muscles: 20 μl each

Intraperitoneal: 10 μl

Diaphragm muscle: 10 μl

Face muscles: 20 μl

Tongue: 10 μl

Intracranial (brainstem): 5 μl

Muscles of the thoracic trunk: 10 μl

All of the lentiviral vectors for these experiments were rabies-G pseudotyped so as to achieve retrograde transport of the virus and transduction of motor neurons.

EIAV gene transfer in a mouse model of type I SMA led to widespread expression of the transgene, extending the survival of these mice. SMN immunostaining demonstrated robust expression of the transgene, not only in spinal motor neurons but also in DRG neurons, suggesting that intramuscular injection of Smart2SMN or pONY8.7NCSMN in type I mice leads to transduction of motor neurons and DRG cells by retrograde transport (FIG. 39). No such staining was seen in mice injected with Smart2LacZ (FIG. 39).

Lentiviral vector-mediated expression of SMN gene in SMA type I mice extended the survival of these mice by 35%, compared to control LacZ treated mice, and 50% compared to untreated mice.

### Example 17

### VEGF Gene Delivery Prolongs Survival of SOD1 Transgenic Mice

The effect of the LentiVector® expressing anti-apoptotic molecules, such as XIAP (Aegera Therapeutics Inc.), and neuroprotective molecules, such as VEGF, IGF-I, GDNF, and siRNA strategy on motor neuron survival in the ALS animal models was studied with the aim of preventing or halting the progress of neurodegeneration in motor neurons of ALS patients.

Gene Therapy in SOD1 Transgenic Mice

To test functional efficiency in SOD1 mice, intramuscular injections of Smart2LacZ and Smart2VEGF and Smart2XIAP were performed (Table 6). Three groups were included in the current experiment: The first group of mice received injections of Smart2VEGF (n=7). The second group were injected with LentiVector® expressing anti-apoptotic protein XLAP (n=6). The control group (n=6) was treated with Smart2LacZ vector. Three muscle groups were targeted (Table 1): leg, face and diaphragm muscles.

TABLE 6
In vivo studies in SOD1 transgenic mice.

| | | Volume and site of injections | | | |
|---|---|---|---|---|---|
| | | Leg | Face | Diaphragm | Titres |
| Treatment | No. of mice | (μl) | (μl) | (μl) | (Taqman) |
| Smart2LacZ | 6 | 25 | 10 | 10 | 3.4 × 10⁹ |
| Smart2VEGF | 7 | 25 | 10 | 10 | 2.1 × 10⁹ |
| Smart2XIAP | 6 | 25 | 10 | 10 | 8.9 × 10⁹ |

Smart2hVEGF treatment delayed the onset of the disease and extended the survival of SOD1 transgenic mice, compared to LacZ control mice. The onset of the disease was delayed by an average of 30 days. hVEGF-injected mice survived a minimum of 40 days longer that LacZ group. However, Smart2XIAP did not show any efficacy in SOD1 transgenic mice. VEGF treatment also enhanced the motor function in SOD1 mice, compared to LacZ group. This result was based on rotarod and footprint tests.

Having thus described in detail preferred embodiments of the present invention, it is to be understood that the invention defined by the appended claims is not to be limited to particular details set forth in the above description, as many apparent variations thereof are possible without departing from the spirit or scope of the present invention. Modifications and variations of the method and apparatuses described herein will be obvious to those skilled in the art, and are intended to be encompassed by the following claims.

1. A method of treating motor neuron disease in a patient in need thereof, the method comprising delivering to a target site, a lentiviral vector pseudotyped with a rabies G envelope protein, the lentiviral vector comprising a nucleotide of interest (NOI), wherein the target site is at least part of the central nervous system, and wherein the NOI encodes a gene product that is expressed in the target site, thereby treating motor neuron disease in the patient.

2. The method of claim 1, wherein treating motor neuron disease comprises halting or delaying the degeneration of motor neurons in the patient.

3. The method of claim 1, wherein the delivery to the target site of the lentiviral vector comprising the NOI is by diffusion.

4. The method of claim 1, wherein the delivery to the target site of the lentiviral vector comprising the NOI is via intramuscular or intraparenchymal administration.

5. The method of claim 1, wherein the delivery to the target site of the lentiviral vector comprising the NOI is via retrograde transport.

6. The method of claim 1, wherein the motor neuron disease is ALS (Amyotrophic Lateral Sclerosis) or SMA (Spinal Muscular Atrophy).

7. The method of claim 1, wherein the target site comprises a target cell selected from the group consisting of a sensory neuron, a motor neuron, an astrocyte, an oligodendrocyte, a microglial cell, and an ependymal cell.

8. The method of claim 1, wherein the NOI encodes a neurotrophic or anti-apoptotic gene product.

9. The method of claim 1, wherein the NOI encodes a protein selected from the group consisting of SMN-1, GDNF, IGF-1, VEGF, XIAP, NIAP, and bcl-2.

10. The method of claim 1, wherein the lentiviral vector is pseudotyped with a mutant, variant, fragment or homologue of a rabies G envelope protein.

11. A method of delivering a nucleotide of interest (NOI) to a target site, comprising introducing a lentiviral vector comprising an NOI and pseudotyped with a rabies G envelope protein to the target site, wherein the target site is at least part of the central nervous system.

12. The method of claim 11, wherein the NOI can treat motor neuron disease by halting or delaying the degeneration of motor neurons in a subject.

13. The method of claim 11, wherein the NOI is introduced to the target site by diffusion.

14. The method of claim 11, wherein the NOI is introduced to the target site via intramuscular or intraparenchymal administration of the lentiviral vector.

15. The method of claim 11, wherein the NOI is introduced to the target site by retrograde transport.

16. The method of claim 12, wherein the motor neuron disease is ALS (Amyotrophic Lateral Sclerosis) or SMA (Spinal Muscular Atrophy).

17. The method of claim 11, wherein the target site comprises a target cell selected from the group consisting of a sensory neuron, a motor neuron, an astrocyte, an oligodendrocyte, a microglial cell, and an ependymal cell.

18. The method of claim 11, wherein the NOI encodes a neurotrophic or anti-apoptotic gene product.

19. The method of claim 11, wherein the NOI encodes a protein selected from the group consisting of SMN-1, GDNF, IGF-1, VEGF, XIAP, NIAP, bcl-2, and RARβ2.

20. The method of claim 11, wherein the lentiviral vector is pseudotyped with a mutant, variant, fragment or homologue of a rabies G envelope protein.

21. A method of expressing a nucleotide of interest (NOI) in a target site, comprising introducing a lentiviral vector comprising an NOI and pseudotyped with a rabies G envelope protein to the target site, wherein the target site is at least part of the central nervous system, and wherein the NOI encodes a gene product that is expressed in the target site.

22. The method of claim 21, wherein expression of the gene product can treat motor neuron disease by halting or delaying the degeneration of motor neurons in a subject.

23. The method of claim 21, wherein the NOI is introduced to the target site by diffusion.

24. The method of claim 21, wherein the NOI is introduced to the target site via intramuscular or intraparenchymal administration of the lentiviral vector.

25. The method of claim 21, wherein the NOI is introduced to the target site by retrograde transport.

26. The method of claim 22, wherein the motor neuron disease is ALS (Amyotrophic Lateral Sclerosis) or SMA (Spinal Muscular Atrophy).

27. The method of claim 21, wherein the target site comprises a target cell selected from the group consisting of a sensory neuron, a motor neuron, an astrocyte, an oligodendrocyte, a microglial cell, and an ependymal cell.

28. The method of claim 21, wherein the NOI encodes a neurotrophic or anti-apoptotic gene product.

29. The method of claim 21, wherein the NOI encodes a protein selected from the group consisting of SMN-1, GDNF, IGF-1, VEGF, XIAP, NIAP, bcl-2, and RARβ2.

30. The method of claim 21, wherein the lentiviral vector is pseudotyped with a mutant, variant, fragment or homologue of a rabies G envelope protein.

31. The method of claim 21, wherein expression of the gene product treats or prevents pain associated with a neurological disorder or injury.

32. A method of treating motor neuron disease in a patient in need thereof, the method comprising delivering to a target site, a lentiviral vector pseudotyped with a rabies G envelope protein, the lentiviral vector comprising a nucleotide of interest (NOI), wherein the target site is at least part of the central nervous system, and wherein the NOI encodes a gene product that is expressed in the target site, thereby treating motor neuron disease in the patient.

33. The method of claim 32, wherein treating motor neuron disease comprises halting or delaying the degeneration of motor neurons in the patient.

34. The method of claim 32, wherein the delivery to the target site of the lentiviral vector comprising the NOI is by diffusion.

35. The method of claim 32, wherein the delivery to the target site of the lentiviral vector comprising the NOI is via intramuscular or intraparenchymal administration.

36. The method of claim 32, wherein the delivery to the target site of the lentiviral vector comprising the NOI is via retrograde transport.

37. The method of claim 32, wherein the motor neuron disease is ALS (Amyotrophic Lateral Sclerosis) or SMA (Spinal Muscular Atrophy).

38. The method of claim 32, wherein the target site comprises a target cell selected from the group consisting of a sensory neuron, a motor neuron, an astrocyte, an oligodendrocyte, a microglial cell, and an ependymal cell.

39. The method of claim 32, wherein the NOI encodes a neurotrophic or anti-apoptotic gene product.

40. The method of claim 32, wherein the NOI encodes a protein selected from the group consisting of SMN-1, GDNF, IGF-1, VEGF, XIAP, NIAP, and bcl-2.

41. The method of claim 32, wherein the lentiviral vector is pseudotyped with a mutant, variant, fragment or homologue of a rabies G envelope protein.

42. A method of delivering a nucleotide of interest (NOI) to a target site, comprising introducing a lentiviral vector comprising an NOI and pseudotyped with a rabies G envelope protein to the target site, wherein the target site is at least part of the central nervous system.

43. The method of claim 42, wherein the NOI can treat motor neuron disease by halting or delaying the degeneration of motor neurons in a subject.

44. The method of claim 42, wherein the NOI is introduced to the target site by diffusion.

45. The method of claim 42, wherein the NOI is introduced to the target site via intramuscular or intraparenchymal administration of the lentiviral vector.

46. The method of claim 42, wherein the NOI is introduced to the target site by retrograde transport.

47. The method of claim 46, wherein the motor neuron disease is ALS (Amyotrophic Lateral Sclerosis) or SMA (Spinal Muscular Atrophy).

48. The method of claim 42, wherein the target site comprises a target cell selected from the group consisting of a sensory neuron, a motor neuron, an astrocyte, an oligodendrocyte, a microglial cell, and an ependymal cell.

49. The method of claim 42, wherein the NOI encodes a neurotrophic or anti-apoptotic gene product.

50. The method of claim 42, wherein the NOI encodes a protein selected from the group consisting of SMN-1, GDNF, IGF-1, VEGF, XIAP, NIAP, bcl-2, and RARβ2.


SEQUENCE LISTING (19 sequences)

SEQ ID NO: 1 - DNA, 10998 bp, Artificial Sequence
  Description of Artificial Sequence Synthetic nucleotide construct pONY8Z sequence
agatcttgaataataaaatgtgtgtttgtccgaaatacgcgttttgagatttctgtcgccgactaaattcatgtcgcgcgatagtggtgtttatcgccgatagagatggcgatattggaaaaattgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcgtaatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgggcactcagattctgcggtctgagtcccttctctgctgggctgaaaaggcctttgtaataaatataattctctactcagtccctgtctctagtttgtctgttcgagatcctacagttggcgcccgaacagggacctgagaggggcgcagaccctacctgttgaacctggctgatcgtaggatccccgggacagcagaggagaacttacagaagtcttctggaggtgttcctggccagaacacaggaggacaggtaagattgggagaccctttgacattggagcaaggcgctcaagaagttagagaaggtgacggtacaagggtctcagaaattaactactggtaactgtaattgggcgctaagtctagtagacttatttcatgataccaactttgtaaaagaaaaggactggcagctgagggatgtcattccattgctggaagatgtaactcagacgctgtcaggacaagaaagagaggcctttgaaagaacatggtgggcaatttctgctgtaaagatgggcctccagattaataatgtagtagatggaaaggcatcattccagctcctaagagcgaaatatgaaaagaagactgctaataaaaagcagtctgagccctctgaagaatatctctagaactagtggatcccccgggctgcaggagtggggaggcacgatggccgctttggtcgaggcggatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcatgtacggtgggaggtctatataagcagagctcgtttagtgaaccgtcagatcgcctggagacgccatccacgctgttttgacctccatagaagacaccgggaccgatccagcctccgcggccccaagcttcagctgctcgaggatctgcggatccggggaattccccagtctcaggatccaccatgggggatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatccgcagatccggctgtggaatgtgtgtcagttagggtgtggaaagtccccaggctccccagcaggcagaagtatgcaaagcatgcctgcaggaattcgatatcaagcttatcgataccgtcgacctcgagggggggcccggtacccagcttttgttccctttagtgagggttaattgcgcgggaagtatttatcactaatcaagcacaagtaatacatgagaaacttttactacagcaagcacaatcctccaaaaaattttgtttttacaaaatccctggtgaacatgattggaagggacctactagggtgctgtggaagggtgatggtgcagtagtagttaatgatgaaggaaagggaataattgctgtaccattaaccaggactaagttactaataaaaccaaattgagtattgttgcaggaagcaagacccaactaccattgtcagctgtgtttcctgacctcaatatttgttataaggtttgatatgaatcccagggggaatctcaacccctattacccaacagtcagaaaaatctaagtgtgaggagaacacaatgtttcaaccttattgttataataatgacagtaagaacagcatggcagaatcgaaggaagcaagagaccaagaatgaacctgaaagaagaatctaaagaagaaaaaagaagaaatgactggtggaaaataggtatgtttctgttatgcttagcaggaactactggaggaatactttggtggtatgaaggactcccacagcaacattatatagggttggtggcgatagggggaagattaaacggatctggccaatcaaatgctatagaatgctggggttccttcccggggtgtagaccatttcaaaattacttcagttatgagaccaatagaagcatgcatatggataataatactgctacattattagaagctttaaccaatataactgctctataaataacaaaacagaattagaaacatggaagttagtaaagacttctggcataactcctttacctatttcttctgaagctaacactggactaattagacataagagagattttggtataagtgcaatagtggcagctattgtagccgctactgctattgctgctagcgctactatgtcttatgttgctctaactgaggttaacaaaataatggaagtacaaaatcatacttttgaggtagaaaatagtactctaaatggtatggatttaatagaacgacaaataaagatattatatgctatgattcttcaaacacatgcagatgttcaactgttaaaggaaagacaacaggtagaggagacatttaatttaattggatgtatagaaagaacacatgtattttgtcatactggtcatccctggaatatgtcatggggacatttaaatgagtcaacacaatgggatgactgggtaagcaaaatggaagatttaaatcaagagatactaactacacttcatggagccaggaacaatttggcacaatccatgataacattcaatacaccagatagtatagctcaatttggaaaagacctttggagtcatattggaaattggattcctggattgggagcttccattataaaatatatagtgatgtttttgcttatttatttgttactaacctcttcgcctaagatcctcagggccctctggaaggtgaccagtggtgcagggtcctccggcagtcgttacctgaagaaaaaattccatcacaaacatgcatcgcgagaagacacctgggaccaggcccaacacaacatacacctagcaggcgtgaccggtggatcaggggacaaatactacaagcagaagtactccaggaacgactggaatggagaatcagaggagtacaacaggcggccaaagagctgggtgaagtcaatcgaggcatttggagagagctatatttccgagaagaccaaaggggagatttctcagcctggggcggctatcaacgagcacaagaacggctctggggggaacaatcctcaccaagggtccttagacctggagattcgaagcgaaggaggaaacatttatgactgttgcattaaagcccaagaaggaactctcgctatcccttgctgtggatttcccttatggctattttggggactagtaattatagtaggacgcatagcaggctatggattacgtggactcgctgttataataaggatttgtattagaggcttaaatttgatatttgaaataatcagaaaaatgcttgattatattggaagagctttaaatcctggcacatctcatgtatcaatgcctcagtatgtttagaaaaacaaggggggaactgtggggtttttatgaggggttttataaatgattataagagtaaaaagaaagttgctgatgctctcataaccttgtataacccaaaggactagctcatgttgctaggcaactaaaccgcaataaccgcatttgtgacgcgagttccccattggtgacgcgttaacttcctgtttttacagtatataagtgcttgtattctgacaattgggcactcagattctgcggtctgagtcccttctctgctgggctgaaaaggcctttgtaataaatataattctctactcagtccctgtctctagtttgtctgttcgagatcctacagagctcatgccttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccaacctggcttatcgaaattaatacgactcactatagggagaccggc

SEQ ID NO: 2 - DNA, 8531 bp, Artificial Sequence
  Description of Artificial Sequence Synthetic nucleotide construct pONY8G sequence
agatcttgaataataaaatgtgtgtttgtccgaaatacgcgttttgagatttctgtcgccgactaaattcatgtcgcgcgatagtggtgtttatcgccgatagagatggcgatattggaaaaattgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcgtaatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgggcactcagattctgcggtctgagtcccttctctgctgggctgaaaaggcctttgtaataaatataattctctactcagtccctgtctctagtttgtctgttcgagatcctacagttggcgcccgaacagggacctgagaggggcgcagaccctacctgttgaacctggctgatcgtaggatccccgggacagcagaggagaacttacagaagtcttctggaggtgttcctggccagaacacaggaggacaggtaagattgggagaccctttgacattggagcaaggcgctcaagaagttagagaaggtgacggtacaagggtctcagaaattaactactggtaactgtaattgggcgctaagtctagtagacttatttcatgataccaactttgtaaaagaaaaggactggcagctgagggatgtcattccattgctggaagatgtaactcagacgctgtcaggacaagaaagagaggcctttgaaagaacatggtgggcaatttctgctgtaaagatgggcctccagattaataatgtagtagatggaaaggcatcattccagctcctaagagcgaaatatgaaaagaagactgctaataaaaagcagtctgagccctctgaagaatatctctagaactagtggatcccccgggctgcaggagtggggaggcacgatggccgctttggtcgaggcggatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcatgtacggtgggaggtctatataagcagagctcgtttagtgaaccgtcagatcgcctggagacgccatccacgctgttttgacctccatagaagacaccgggaccgatccagcctccgcggccccaagcttgttgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagagtcgacctgcaggcatgcaagcttcagctgctcgagggggggcccggtacccagcttttgttccctttagtgagggttaattgcgcgggaagtatttatcactaatcaagcacaagtaatacatgagaaacttttactacagcaagcacaatcctccaaaaaattttgtttttacaaaatccctggtgaacatgattggaagggacctactagggtgctgtggaagggtgatggtgcagtagtagttaatgatgaaggaaagggaataattgctgtaccattaaccaggactaagttactaataaaaccaaattgagtattgttgcaggaagcaagacccaactaccattgtcagctgtgtttcctgacctcaatatttgttataaggtttgatatgaatcccagggggaatctcaacccctattacccaacagtcagaaaaatctaagtgtgaggagaacacaatgtttcaaccttattgttataataatgacagtaagaacagcatggcagaatcgaaggaagcaagagaccaagaatgaacctgaaagaagaatctaaagaagaaaaaagaagaaatgactggtggaaaataggtatgtttctgttatgcttagcaggaactactggaggaatactttggtggtatgaaggactcccacagcaacattatatagggttggtggcgatagggggaagattaaacggatctggccaatcaaatgctatagaatgctggggttccttcccggggtgtagaccatttcaaaattacttcagttatgagaccaatagaagcatgcatatggataataatactgctacattattagaagctttaaccaatataactgctctataaataacaaaacagaattagaaacatggaagttagtaaagacttctggcataactcctttacctatttcttctgaagctaacactggactaattagacataagagagattttggtataagtgcaatagtggcagctattgtagccgctactgctattgctgctagcgctactatgtcttatgttgctctaactgaggttaacaaaataatggaagtacaaaatcatacttttgaggtagaaaatagtactctaaatggtatggatttaatagaacgacaaataaagatattatatgctatgattcttcaaacacatgcagatgttcaactgttaaaggaaagacaacaggtagaggagacatttaatttaattggatgtatagaaagaacacatgtattttgtcatactggtcatccctggaatatgtcatggggacatttaaatgagtcaacacaatgggatgactgggtaagcaaaatggaagatttaaatcaagagatactaactacacttcatggagccaggaacaatttggcacaatccatgataacattcaatacaccagatagtatagctcaatttggaaaagacctttggagtcatattggaaattggattcctggattgggagcttccattataaaatatatagtgatgtttttgcttatttatttgttactaacctcttcgcctaagatcctcagggccctctggaaggtgaccagtggtgcagggtcctccggcagtcgttacctgaagaaaaaattccatcacaaacatgcatcgcgagaagacacctgggaccaggcccaacacaacatacacctagcaggcgtgaccggtggatcaggggacaaatactacaagcagaagtactccaggaacgactggaatggagaatcagaggagtacaacaggcggccaaagagctgggtgaagtcaatcgaggcatttggagagagctatatttccgagaagaccaaaggggagatttctcagcctggggcggctatcaacgagcacaagaacggctctggggggaacaatcctcaccaagggtccttagacctggagattcgaagcgaaggaggaaacatttatgactgttgcattaaagcccaagaaggaactctcgctatcccttgctgtggatttcccttatggctattttggggactagtaattatagtaggacgcatagcaggctatggattacgtggactcgctgttataataaggatttgtattagaggcttaaatttgatatttgaaataatcagaaaaatgcttgattatattggaagagctttaaatcctggcacatctcatgtatcaatgcctcagtatgtttagaaaaacaaggggggaactgtggggtttttatgaggggttttataaatgattataagagtaaaaagaaagttgctgatgctctcataaccttgtataacccaaaggactagctcatgttgctaggcaactaaaccgcaataaccgcatttgtgacgcgagttccccattggtgacgcgttaacttcctgtttttacagtatataagtgcttgtattctgacaattgggcactcagattctgcggtctgagtcccttctctgctgggctgaaaaggcctttgtaataaatataattctctactcagtccctgtctctagtttgtctgttcgagatcctacagagctcatgccttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccaacctggcttatcgaaattaatacgactcactatagggagaccggc

SEQ ID NO: 3 - protein, 524 aa, Rabies virus
MVPQALLFVPLLVFPLCFGKFPIYTIPDKLGPWSPIDIHHLSCPNNLVVEDEGCTNLSGFSYMELKVGYILAIKMNGFTCTGVVTEAETYTNFVGYVTTTFKRKHFRPTPDACRAAYNWKMAGDPRYEESLHNPYPDYRWLRTVKTTKESLVIISPSVADLDPYDRSLHSRVFPSGKCSGVAVSSTYCSTNHDYTIWMPENPRLGMSCDIFTNSRGKRASKGSETCGFVDERGLYKSLKGACKLKLCGVLGLRLMDGTWVAMQTSNETKWCPPDQLVNLHDFRSDEIEHLVVEELVRKREECLDALESIMTTKSVSFRRLSHLRKLVPGFGKAYTIFNKTLMEADAHYKSVRTWNEILPSKGCLRVGGRCHPHVNGVFFNGIILGPDGNVLIPEMQSSLLQQHMELLESSVIPLVHPLADPSTVFKDGDEAEDFVEVHLPDVHNQVSGVDLGLPNWGKYVLLSAGALTALMLIIFLMTCCRRVNRSEPTQHNLRGTGREVSVTPQSGKIISSWESHKSGGETRL

SEQ ID NO: 4 - DNA, 1650 bp, Rabies virus
aggaaagatggttcctcaggctctcctgtttgtaccccttctggtttttccattgtgttttgggaaattccctatttacacgatcccagacaagcttggtccctggagcccgattgacatacatcacctcagctgcccaaacaatttggtagtggaggacgaaggatgcaccaacctgtcagggttctcctacatggaacttaaagttggatacatcttagccataaaaatgaacgggttcacttgcacaggcgttgtgacggaggctgaaacctacactaacttcgttggttatgtcacaaccacgttcaaaagaaagcatttccgcccaacaccagatgcatgtagagccgcgtacaactggaagatggccggtgaccccagatatgaagagtctctacacaatccgtaccctgactaccgctggcttcgaactgtaaaaaccaccaaggagtctctcgttatcatatctccaagtgtagcagatttggacccatatgacagatcccttcactcgagggtcttccctagcgggaagtgctcaggagtagcggtgtcttctacctactgctccactaaccacgattacaccatttggatgcccgagaatccgagactagggatgtcttgtgacatttttaccaatagtagagggaagagagcatccaaagggagtgagacttgcggctttgtagatgaaagaggcctatataagtctttaaaaggagcatgcaaactcaagttatgtggagttctaggacttagacttatggatggaacatgggtcgcgatgcaaacatcaaatgaaaccaaatggtgccctcccgatcagttggtgaacctgcacgactttcgctcagacgaaattgagcaccttgttgtagaggagttggtcaggaagagagaggagtgtctggatgcactagagtccatcatgacaaccaagtcagtgagtttcagacgtctcagtcatttaagaaaacttgtccctgggtttggaaaagcatataccatattcaacaagaccttgatggaagccgatgctcactacaagtcagtcagaacttggaatgagatcctcccttcaaaagggtgtttaagagttggggggaggtgtcatcctcatgtgaacggggtgtttttcaatggtataatattaggacctgacggcaatgtcttaatcccagagatgcaatcatccctcctccagcaacatatggagttgttggaatcctcggttatcccccttgtgcaccccctggcagacccgtctaccgttttcaaggacggtgacgaggctgaggattttgttgaagttcaccttcccgatgtgcacaatcaggtctcaggagttgacttgggtctcccgaactgggggaagtatgtattactgagtgcaggggccctgactgccttgatgttgataattttcctgatgacatgttgtagaagagtcaatcgatcagaacctacgcaacacaatctcagagggacagggagggaggtgtcagtcactccccaaagcgggaagatcatatcttcatgggaatcacacaagagtgggggtgagaccagactgtgaggactggccgtcctttcaacgatccaagtcctgaagatcacctccccttggggggttctttttaaaaa

SEQ ID NO: 5 - DNA, 8870 bp, Artificial Sequence
  Description of Artificial Sequence Synthetic nucleotide construct pONY8.1Z sequence
agatcttgaataataaaatgtgtgtttgtccgaaatacgcgttttgagatttctgtcgccgactaaattcatgtcgcgcgatagtggtgtttatcgccgatagagatggcgatattggaaaaattgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcgtaatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgggcactcagattctgcggtctgagtcccttctctgctgggctgaaaaggcctttgtaataaatataattctctactcagtccctgtctctagtttgtctgttcgagatcctacagttggcgcccgaacagggacctgagaggggcgcagaccctacctgttgaacctggctgatcgtaggatccccgggacagcagaggagaacttacagaagtcttctggaggtgttcctggccagaacacaggaggacaggtaagattgggagaccctttgacattggagcaaggcgctcaagaagttagagaaggtgacggtacaagggtctcagaaattaactactggtaactgtaattgggcgctaagtctagtagacttatttcatgataccaactttgtaaaagaaaaggactggcagctgagggatgtcattccattgctggaagatgtaactcagacgctgtcaggacaagaaagagaggcctttgaaagaacatggtgggcaatttctgctgtaaagatgggcctccagattaataatgtagtagatggaaaggcatcattccagctcctaagagcgaaatatgaaaagaagactgctaataaaaagcagtctgagccctctgaagaatatctctagaactagtggatcccccgggctgcaggagtggggaggcacgatggccgctttggtcgaggcggatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcatgtacggtgggaggtctatataagcagagctcgtttagtgaaccgtcagatcgcctggagacgccatccacgctgttttgacctccatagaagacaccgggaccgatccagcctccgcggccccaagcttcagctgctcgaggatctgcggatccggggaattccccagtctcaggatccaccatgggggatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatccgcagatccggctgtggaatgtgtgtcagttagggtgtggaaagtccccaggctccccagcaggcagaagtatgcaaagcatgcctgcaggaattcgatatcaagcttatcgataccgtcgaattggaagagctttaaatcctggcacatctcatgtatcaatgcctcagtatgtttagaaaaacaaggggggaactgtggggtttttatgaggggttttataaatgattataagagtaaaaagaaagttgctgatgctctcataaccttgtataacccaaaggactagctcatgttgctaggcaactaaaccgcaataaccgcatttgtgacgcgagttccccattggtgacgcgttaacttcctgtttttacagtatataagtgcttgtattctgacaattgggcactcagattctgcggtctgagtcccttctctgctgggctgaaaaggcctttgtaataaatataattctctactcagtccctgtctctagtttgtctgttcgagatcctacagagctcatgccttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccaacctggcttatcgaaattaatacgactcactatagggagaccggc

SEQ ID NO: 6 - DNA, 24 bp, Unknown Organism
  Description of Unknown Organism Illustrative DNA sequence
atttacacgatactagacaagctt

SEQ ID NO: 7 - protein, 8 aa, Unknown Organism
  Description of Unknown Organism Illustrative amino acid sequence
IYTILDKL

SEQ ID NO: 8 - DNA, 24 bp, Artificial Sequence
  Description of Artificial Sequence Synthetic DNA sequence of the present invention
atttacacgatcccagacaagctt

SEQ ID NO: 9 - protein, 8 aa, Artificial Sequence
  Description of Artificial Sequence Synthetic amino acid sequence of the present invention
IYTIPDKL

SEQ ID NO: 10 - DNA, 24 bp, Artificial Sequence
  Description of Artificial Sequence Synthetic primer
cgttgctgcataaaccgactacac

SEQ ID NO: 11 - DNA, 22 bp, Artificial Sequence
  Description of Artificial Sequence Synthetic primer
tgcagaggatgatgctcgtgac

SEQ ID NO: 12 - DNA, 1650 bp, Rabies virus
aggaaagatggttcctcaggctctcctgtttgtaccccttctggtttttccattgtgttttgggaaattccctatttacacgatactagacaagcttggtccctggagcccgattgacatacatcacctcagctgcccaaacaatttggtagtggaggacgaaggatgcaccaacctgtcagggttctcctacatggaacttaaagttggatacatcttagccataaaaatgaacgggttcacttgcacaggcgttgtgacggaggctgaaacctacactaacttcgttggttatgtcacaaccacgttcaaaagaaagcatttccgcccaacaccagatgcatgtagagccgcgtacaactggaagatggccggtgaccccagatatgaagagtctctacacaatccgtaccctgactaccgctggcttcgaactgtaaaaaccaccaaggagtctctcgttatcatatctccaagtgtagcagatttggacccatatgacagatcccttcactcgagggtcttccctagcgggaagtgctcaggagtagcggtgtcttctacctactgctccactaaccacgattacaccatttggatgcccgagaatccgagactagggatgtcttgtgacatttttaccaatagtagagggaagagagcatccaaagggagtgagacttgcggctttgtagatgaaagaggcctatataagtctttaaaaggagcatgcaaactcaagttatgtggagttctaggacttagacttatggatggaacatgggtcgcgatgcaaacatcaaatgaaaccaaatggtgccctcccgatcagttggtgaacctgcacgactttcgctcagacgaaattgagcaccttgttgtagaggagttggtcaggaagagagaggagtgtctggatgcactagagtccatcatgacaaccaagtcagtgagtttcagacgtctcagtcatttaagaaaacttgtccctgggtttggaaaagcatataccatattcaacaagaccttgatggaagccgatgctcactacaagtcagtcagaacttggaatgagatcctcccttcaaaagggtgtttaagagttggggggaggtgtcatcctcatgtgaacggggtgtttttcaatggtataatattaggacctgacggcaatgtcttaatcccagagatgcaatcatccctcctccagcaacatatggagttgttggaatcctcggttatcccccttgtgcaccccctggcagacccgtctaccgttttcaaggacggtgacgaggctgaggattttgttgaagttcaccttcccgatgtgcacaatcaggtctcaggagttgacttgggtctcccgaactgggggaagtatgtattactgagtgcaggggccctgactgccttgatgttgataattttcctgatgacatgttgtagaagagtcaatcgatcagaacctacgcaacacaatctcagagggacagggagggaggtgccagtcactccccaaagcgggaagatcatatcttcatgggaatcacacaagagtgggggtgagaccagactgtgaggactggccgtcctttcaacgatccaagtcctgaagatcacctccccttggggggttctttttaaaaa

SEQ ID NO: 13 - protein, 525 aa, Rabies virus
MVPQALLFVPLLVFPLCFGKFPIYTILDKLGPWSPIDIHHLSCPNNLVVEDEGCTNLSGFSYMELKVGYILAIKMNGFTCTGVVTEAETYTNFVGYVTTTFKRKHFRPTPDACRAAYNWKMAGDPRYEESLHNPYPDYRWLRTVKTTKESLVIISPSVADLIDPYDRSLHSRVFPSGKCSGVAVSSTYCSTNHDYTIWMPENPRLGMSCDIFTNSRGKRASKGSETCGFVDERGLYKSLKGACKLKLCGVLGLRLMDGTWVAMQTSNETKWCPPDQLVNLHDFRSDEIEHLVVEELVRKREECLDALESIMTTKSVSFRRLSHLRKLVPGFGKAYTIFNKTLMEADAHYKSVRTWNEILPSKGCLRVGGRCHPHVNGVFFNGIILGPDGNVLIPEMQSSLLQQHMELLESSVIPLVHPLADPSTVFKDGDEAEDFVEVHLPDVHNQVSGVDLGLPNWGKYVLLSAGALTALMLIIFLMTCCRRVNRSEPTQHNLRGTGREVSVTPQSGKIISSWESHKSGGETRL

SEQ ID NO: 14 - DNA, 1575 bp, Rabies virus
atggttcctcaggctctcctgtttgtaccccttctggtttttccattgtgttttgggaaattccctatttacacgatcccagacaagcttggtccctggagcccgattgacatacatcacctcagctgcccaaacaatttggtagtggaggacgaaggatgcaccaacctgtcagggttctcctacatggaacttaaagttggatacatcttagccataaaaatgaacgggttcacttgcacaggcgttgtgacggaggctgaaacctacactaacttcgttggttatgtcacaaccacgttcaaaagaaagcatttccgcccaacaccagatgcatgtagagccgcgtacaactggaagatggccggtgaccccagatatgaagagtctctacacaatccgtaccctgactaccgctggcttcgaactgtaaaaaccaccaaggagtctctcgttatcatatctccaagtgtagcagatttggacccatatgacagatcccttcactcgagggtcttccctagcgggaagtgctcaggagtagcggtgtcttctacctactgctccactaaccacgattacaccatttggatgcccgagaatccgagactagggatgtcttgtgacatttttaccaatagtagagggaagagagcatccaaagggagtgagacttgcggctttgtagatgaaagaggcctatataagtctttaaaaggagcatgcaaactcaagttatgtggagttctaggacttagacttatggatggaacatgggtcgcgatgcaaacatcaaatgaaaccaaatggtgccctcccgatcagttggtgaacctgcacgactttcgctcagacgaaattgagcaccttgttgtagaggagttggtcaggaagagagaggagtgtctggatgcactagagtccatcatgacaaccaagtcagtgagtttcagacgtctcagtcatttaagaaaacttgtccctgggtttggaaaagcatataccatattcaacaagaccttgatggaagccgatgctcactacaactcagtcatgacttggaatgagatcctcccctcaaaagggtgtttaagagttggggggaggtgtcatcctcatgtgaacggggtgtttttcaatggtataatattaggacctgacggcaatgtcttaatcccagagatgcaatcatccctcctccagcaacatatggagttgttggaatcctcggttatcccccttgtgcaccccctggcagacccgtctaccgttttcaaggacggtgacgaggctgaggattttgttgaagttcaccttcccgatgtgcacaatcaggtctcaggagttgacttgggtctcccgaactgggggaagtatgtattactgagtgcaggggccctgactgccttgatgttgataattttcctgatgacatgttgtagaagagtcaatcgatcagaacctacgcaacacaatctcagagggacagggagggaggtgtcagtcactccccaaagcgggaagatcatatcttcatgggaatcacacaagagtgggggtgagaccagactgtga

SEQ ID NO: 15 - DNA, 1575 bp, Rabies virus
atggttcctcaggttcttttgtttgtactccttctgggtttttcgttgtgtttcgggaagttccccatttacacgataccagacaaacttggtccctggagccctattgacatacaccatctccgctgtccaaataacctggttgtggaggatgaaggatgtatcaacctgtccgggttctcctacatggaactcaaagtgggatacatctcagccatcaaagtgaacgggttcacttgcacaggtgttgtgacagaggcagagacctacaccaactttgttggttatgtcacaaccacattcaagagaaagcatttccgccccaccccagacgcatgtagagccgcgtataactggaagatggccggtgaccccagatatgaagagtccctacaaaatccataccccgactaccactggcttcgaactgtaagaaccaccaaagagtccctcattatcatatccccaagtgtgacagatttggacccatatgacaaatcccttcactcaagggtcttccctggcggaaagtgctcaggaataacggtgtcctctacctactgctcaactaaccatgattacaccatttggatgcccgagaatccgagaccagggacaccttgtgacatttttaccaatagcagagggaagagagcatccaacgggaacaagacttgcggctttgtggatgaaagaggcctgtataagtctctaaaaggagcatgcaggctcaagttatgtggagttcttggacttagacttatggatggaacatgggtcgcgatgcaaacatcagatgagaccaaatggtgctctccagatcagttggtgaatttgcacgactttcgctcagacgagattgagcatctcgttgtggaggagttagtcaagaaaagagaggaatgtctggatacattagagtccatcatgaccaccaagtcagtaagtttcagacgtctcagtcacctgagaaaacttgtcccagggtttggaaaagcatataccatattcaacaaaaccttgatggaggctgatgctcactacaagtcagtccggacctggaatgagatcatcccctcaaaagggtgtttgaaagttggaggaaggtgccatcctcatgtgaacggggtgtttttcaatggtataatattagggcctgacgaccgtgtcctaatcccagagatgcaatcatccctcctccggcaacatatggagttgttggaatcttcagttatccccctgatgcaccccctggctgacccttctacagttttcaaagaaggtgatgaggctgaggattttgttgaagttcacctccccgatgtgtacaaacagatctcaggggttgacctgggtctcccgaactggggaaagtatgtattgatgactgcaggggccatgattggcctggtgttgatattttccctaatgacatggtgcagaagagccaatcgaccagaatcgaaacaacgcagttttggagggacaggggggaatgtgtcagtcacttcccaaagcggaaaagtcataccttcatgggaatcatataagagtggaggtgagatcagactgtga

SEQ ID NO: 16 - DNA, 67 bp, Artificial Sequence
  Description of Artificial Sequence Synthetic primer
ctacaactcagtcatgacttggaatgagatcctcccctcaaaagggtgtttaagagttggggggagg

SEQ ID NO: 17 - DNA, 69 bp, Artificial Sequence
  Description of Artificial Sequence Synthetic primer
ccttttgaggggaggatctcattccaagtcatgactgagttgtagtgagcatcggcttccatcaaggtc

SEQ ID NO: 18 - DNA, 20 bp, Artificial Sequence
  Description of Artificial Sequence Synthetic primer
accgtccttgacacgaagct

SEQ ID NO: 19 - DNA, 20 bp, Artificial Sequence
  Description of Artificial Sequence Synthetic primer
gggggaggtgtgggaggttt